(19) United States
(12) Patent Application Publication
(10) Pub. No.: US 2016/0222370 A1
(43) Pub. Date: Aug. 4, 2016

(54) Recombinant Yeast Host Cell With Fe-S Cluster Proteins And Methods Of Using Thereof

(71) Applicant: Butamax Advanced Biofuels LLC, Wilmington, DE (US)

(72) Inventors: Larry Cameron ANTHONY, Aston, PA (US); Dennis Flint, Newark, DE (US); Wonchul Suh, Hockessin, DE (US); Rick W. Ye, Hockessin, DE (US); Steven Cary Rothman, Wilmington, DE (US); Jean-Francois Tomb, Wilmington, DE (US)

(21) Appl. No.: 15/016,759

(22) Filed: Feb. 5, 2016

Related U.S. Application Data
(63) Continuation-in-part of application No. 14/873,956, filed on Oct. 2, 2015.
Application No. 14/873,956 is a division of application No. 12/569,069, filed on Sep. 29, 2009, now Pat. No. 9,206,447.
Application No. 12/569,069 is a continuation-in-part of application No. 12/569,636, filed on Sep. 29, 2009.
(60) Provisional application No. 61/100,801, filed on Sep. 29, 2008.
(60) Provisional application No. 61/100,806, filed on Sep. 29, 2008.
(60) Provisional application No. 61/100,792, filed on Sep. 29, 2008.

(51) Int. Cl. C12N 9/88; C12P 7/16

(52) U.S. Cl. CPC C12N 9/88; C12P 7/16; C12Y 402/01009

(57) ABSTRACT

Yeast strains were engineered that have increased activity of heterologous proteins that require binding of an Fe—S cluster for their activity. The yeast strains have reduced activity of an endogenous Fe—S protein. Activities of heterologous bacterial, fungal or plant 2Fe-2S dihydroxy-acid dehydratases and Fe—S propanediol dehydratase reactivase were increased for increased production of products made using biosynthetic pathways including these enzymes, such as valine, isoleucine, leucine, pantothenic acid (vitamin B5), isobutanol, 2-butanone and 2-butanol.

### CROSS REFERENCE TO RELATED APPLICATIONS

This application is a continuation-in-part application of U.S. patent application Ser. No. 14/873,956, filed on Oct. 2, 2015, which is a division of U.S. patent application Ser. No. 12/569,069, filed on Sept. 29, 2009, which is related to and claims the benefit of priority to U.S. Provisional Application Nos. 61/100,801, filed on Sept. 29, 2008 and U.S. Provisional Application No. 61/100,806, filed on Sep. 29, 2008, and a continuation-in-part of U.S. application Ser. No. 12/569,636, filed Sep. 29, 2009, which claims the benefit of priority to U.S. Provisional Application No. 61/100,792, filed Sep. 29, 2008. The entirety of each is hereby incorporated by reference.

### FIELD OF THE INVENTION

The invention relates to the field of industrial microbiology and the expression of proteins that require an iron-sulfur cluster for activity. More specifically, expression of heterologous Fe—S protein activity in yeast cells is improved through specific host gene inactivation.

### BACKGROUND OF THE INVENTION

Engineering of yeast for fermentative production of commercial products is an active and growing field. Enzymatic pathways engineered for biosynthesis of some products include enzymes that require binding of an iron-sulfur (Fe—S) cluster for activity. Dihydroxy-acid dehydratase (DHAD) is one example. DHAD is part of naturally occurring biosynthetic pathways producing valine, isoleucine, leucine and pantothenic acid (vitamin B5). Increased expression of DHAD activity is desired for enhanced microbial production of branched chain amino acids or of pantothenic acid. In addition, DHAD catalyzed conversion of 2,3-dihydroxyisovalerate to α-ketoisovalerate is a common step in the multiple isobutanol biosynthetic pathways that are disclosed in co-pending US Patent Pub No. US 20070092957 A1. Disclosed therein is engineering of recombinant microorganisms for production of isobutanol, which is useful as a fuel additive and whose availability may reduce the demand for petrochemical fuels.

Diol dehydratase provides an enzyme activity in a biosynthetic pathway for production of 2-butanone and 2-butanol that is disclosed in co-pending US Patent Pub No. US 2007-0292927A1. Disclosed in US Patent Pub No. US20090155870 is a butanediol dehydratase that is useful for expression in this pathway due to its coenzyme B-12 independence. A diol dehydratase reactivase that is an Fe—S cluster protein required for activity of the B12-independent butanediol dehydratase, is also disclosed in US Patent Pub No. US20090155870. 2-Butanone, also referred to as methyl ethyl ketone (MEK), is a widely used solvent, extractant and activator of oxidative reactions, as well as a substrate for chemical synthesis of 2-butanol. 2-butanol is useful as a fuel additive, whose availability may reduce the demand for petrochemical fuels.

For improved production of compounds synthesized in pathways including an Fe—S cluster containing enzyme, it is desirable to provide a host cell capable of expressing high levels of this enzymatic activity in the production host of interest. Whereas a number of commercially relevant bacteria and yeast can express activity of Fe—S cluster containing proteins, this activity is at levels far below what is commercially useful for enhancing introduced biosynthetic pathways. Consequently a need exists for the discovery of host cells capable of expressing activity of Fe—S cluster containing proteins at levels high enough to enhance introduced pathways that have Fe—S requirements. Obtaining high functional expression of heterologous Fe—S cluster containing enzymes is problematic due to the Fe—S cluster requirement, which involves availability and proper loading of the cluster into the apo-protein.

### SUMMARY OF THE INVENTION

Provided herein are recombinant yeast host cells comprising at least one heterologous Fe—S cluster protein wherein the yeast host has reduced expression of at least one endogenous Fe—S cluster protein.

The recombinant yeast cell may be grown under suitable conditions for the production of products including isobutanol, 2-butanol and 2-butanone.

In one aspect, the recombinant yeast cell comprises a disruption in the gene encoding the at least one endogenous Fe—S cluster protein.

In another aspect, the endogenous Fe—S cluster protein is selected from the group consisting of dihydroxy-acid dehydratase, isopropylmalate dehydratase, sulfite reductase, glutamate dehyddrogenase, biotin synthase, aconitase, homoaconitase, lipoate synthase, ferredoxin maturation, NADH ubiquinone oxidoreductase, succinate dehydrogenase, ubiquinol-cytochrome-c reductase, ABC protein Rli1, NTPase Nbp35, and hydrogenase-like protein.

In another aspect, the yeast is selected from the group consisting of Saccharomyces, Schizosaccharomyces, Hansenula, Candida, Kluyveromyces, Yarrowia and Pichia.

In another aspect, the endogenous Fe—S protein is expressed in the mitochondria, and in another embodiment, the endogenous Fe—S cluster protein has an activity selected from the group consisting of: dihydroxy-acid dehydratase and isopropylmalate dehydratase activity.

In another aspect, the host cell is Saccharomyces expressing a gene encoding a polypeptide having the amino acid sequence as set forth in SEQ ID NO:114.

In some embodiments, the at least one heterologous Fe—S cluster protein is selected from the group consisting of bacterial 2Fe-2S dihydroxy-acid dehydratases, fungal 2Fe-2S dihydroxy-acid dehydratases and plant 2Fe-2S dihydroxy-acid dehydratases. In one embodiment, the heterologous bacterial, fungal or plant 2Fe-2S cluster dihydroxy-acid dehydratase is expressed in the cytosol. In one embodiment, the heterologous bacterial, fungal or plant 2Fe-2S cluster dihydroxy-acid dehydratase is a polypeptide having an amino acid sequence that matches the Profile HMM of table 9 with an E value of <10⁻⁵ wherein the polypeptide additionally comprises all three conserved cysteines, corresponding to positions 56, 129, and 201 in the amino acids sequences of the Streptococcus mutans DHAD enzyme corresponding to SEQ ID NO:179. In one embodiment, the heterologous fungal or plant 2Fe-2S cluster dihydroxy-acid dehydratase is a polypeptide having an amino acid sequence that has at least about 95% sequence identity to an amino acid sequence selected from the group consisting of SEQ ID NOs:46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150 and 152. In one embodiment, the heterologous fungal or plant 2Fe-2S cluster dihydroxy-acid dehydratase is a polypeptide having an amino acid sequence that is at least about 90% identical to SEQ ID NO:114 using the Clustal W method of alignment using the default parameters of GAP PENALTY=10, GAP LENGTH PENALTY=0.1, and Gonnet 250 series of protein weight matrix over the full length of the protein sequence. In one embodiment, the heterologous bacterial 2Fe-2S cluster dihydroxy-acid dehydratase is a polypeptide having an amino acid sequence that has at least about 95% sequence identity to an amino acid sequence selected from the group consisting of SEQ ID NOs:175, 177, 179, 181, 183, 185, 187, and 189. In one embodiment, the heterologous bacterial 2Fe-2S cluster dihydroxy-acid dehydratase is a polypeptide having an amino acid sequence that is at least about 90% identical to SEQ ID NO: 179 or 187 using the Clustal W method of alignment using the default parameters of GAP PENALTY=10, GAP LENGTH PENALTY=0.1, and Gonnet 250 series of protein weight matrix over the full length of the protein sequence.

In another aspect, a method for the conversion of 2,3-dihydroxyisovalerate to α-ketoisovalerate is provided, said method comprising:

a) providing (1) a recombinant yeast host cell comprising at least one heterologous gene encoding a 2Fe-2S dihydroxy-acid dehydratase wherein the recombinant yeast host cell has reduced activity of at least one endogenous Fe—S cluster protein; and (2) a source of 2,3-dihydroxyisovalerate; and

b) growing the recombinant host cell of (a) with said source of 2,3-dihydroxyisovalerate under conditions where the 2,3-dihydroxyisovalerate is converted by the host cell to α-ketoisovalerate.

In another aspect, a method for the conversion of 2,3-butanediol to 2-butanone is provided, said method comprising:

a) providing (1) a recombinant yeast host cell comprising at least one heterologus gene encoding a Fe—S propanediol dehydratase reactivase wherein the recombinant yeast host cell has reduced activity of at least one endogenous Fe—S cluster protein; and (2) a source of 2,3-butanediol; and

b) growing the recombinant host cell of (a) with said source of 2,3-butanediol under conditions where 2,3-butanediol is converted by the hots cell to 2-butanone.

Also provided is a method for the production of isobutanol comprising growing a recombinant yeast host cell disclosed herein under conditions wherein isobutanol is produced.

In other embodiments, the at least one heterologous Fe—S cluster protein has Fe—S propanediol dehydratase reactivase activity. In some embodiments, the at least one heterologous Fe—S cluster protein having Fe—S propanediol dehydratase reactivase activity is a propanediol deydratase reactivase having an amino acid sequence that is at least about 90% identical to the amino acid sequence as set forth in SEQ ID NO:44 using the Clustal W method of alignment using the default parameters of GAP PENALTY=10, GAP LENGTH PENALTY=0.1, and Gonnet 250 series of protein weight matrix over the full length of the protein sequence.

In some embodiments, the cell produces 2-butanol, and in some embodiments the cell produces 2-butanone. In some embodiments, the cell comprises a 2-butanol biosynthetic pathway, and in some embodiments, the cell comprises a 2-butanone biosynthetic pathway.

### BRIEF DESCRIPTION OF THE FIGURES AND SEQUENCE DESCRIPTIONS

The invention can be more fully understood from the following detailed description, figures, and the accompanying sequence descriptions, which form a part of this application.

FIG. 1 shows biosynthetic pathways for isobutanol production.

FIG. 2 shows a biosynthetic pathway for 2-butanone and 2-butanol production.

Table 9 is a table of the Profile HMM for dihydroxy-acid dehydratases based on enzymes with assayed function prepared as described in Example 1.

The following sequences conform with 37 C.F.R. 1.821-1.825 (“Requirements for Patent Applications Containing Nucleotide Sequences and/or Amino Acid Sequence Disclosures-the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

TABLE 1
Inactivation target Fe—S protein encoding genes

| | SEQ ID NO: | SEQ ID NO: |
|---|---|---|
| Organism and gene | Nucleic Acid | Peptide |
| Saccharomyces cerevisiae LEU1 | 1 | 2 |
| Schizosaccharomyces pombe LEU1 | 3 | 4 |
| Candida galbrata CBS 138 LEU1 | 5 | 6 |
| Candida albicans SC 5314 LEU1 | 7 | 8 |
| Kluyveromyces lactis LEU1 | 9 | 10 |
| Yarrowia lipolytica LEU1 | 11 | 12 |
| Pichia stipitis LEU1 | 13 | 14 |
| Saccharomyces cerevisiae YJM789 | 111 | 112 |
| ILV3 | | |
| Schizosaccharomyces pombe ILV3 | 93 | 94 |
| Candida galbrata CBS 138 ILV3 | 107 | 108 |
| Candida albicans SC5314 ILV3 | 101 | 102 |
| Kluyveromyces lactis ILV3 | 113 | 114 |
| Yarrowia lipolytica ILV3 | 105 | 106 |
| Pichia stipitis CBS 6054 ILV3 | 103 | 104 |
| Saccharomyces cerevisiae ACO1 | 153 | 154 |
| Schizosaccharomyces pombe | 155 | 156 |
| (chromosome II) ACO1 | | |
| Schizosaccharomyces pombe | 157 | 158 |
| (chromosome I) ACO1 | | |
| Kluyveromyces lactis NRRL Y-1140 | 159 | 160 |
| ACO1 | | |
| Candida albicans SC5314 ACO1 | 161 | 162 |
| Yarrowia lipolytica CLIB122 ACO1 | 163 | 164 |
| Pichia stipitis CBS 6054 ACO1 | 165 | 166 |
| Candida glabrata CBS138 | 167 | 168 |
| (chromosome F) ACO1 | | |
| Candida glabrata CBS138 | 169 | 170 |
| (chromosome D) ACO1 | | |
| Candida glabrata CBS138 | 171 | 172 |
| (chromosome K) ACO1 | | |

TABLE 2
Fungal and plant 2Fe—2S DHADs in addition to those in Table 1

| | SEQ ID NO: | SEQ ID NO: |
|---|---|---|
| Description | Nucleic acid | Peptide |
| Chlamydomonas reinhardtii | 45 | 46 |
| Ostreococcus lucimarinus CCE9901 | 47 | 48 |
| Vitis vinifera | 49 | 50 |
| (Unnamed protein product: | | |
| CAO71581.1) | | |
| Vitis vinifera | 51 | 52 |
| (CAN67446.1) | | |
| Arabidopsis thaliana | 53 | 54 |
| Oryza sativa (indica cultivar-group) | 55 | 56 |
| Physcomitrella patens subsp. patens | 57 | 58 |
| Chaetomium globosum CBS 148.51 | 59 | 60 |
| Neurospora crassa OR74A | 61 | 62 |
| Magnaporthe grisea 70-15 | 63 | 64 |
| Gibberella zeae PH-1 | 65 | 66 |
| Aspergillus niger | 67 | 68 |
| Neosartorya fischeri NRRL 181 | 69 | 70 |
| (XP_001266525.1) | | |
| Neosartorya fischeri NRRL 181 | 71 | 72 |
| (XP_001262996.1) | | |
| Aspergillus niger | 73 | 74 |
| (An03g04520) | | |
| Aspergillus niger | 75 | 76 |
| (An14g03280) | | |
| Aspergillus terreus NIH2624 | 77 | 78 |
| Aspergillus clavatus NRRL 1 | 79 | 80 |
| Aspergillus nidulans FGSC A4 | 81 | 82 |
| Aspergillus oryzae | 83 | 84 |
| Ajellomyces capsulatus NAm1 | 85 | 86 |
| Coccidioides immitis RS | 87 | 88 |
| Botryotinia fuckeliana B05.10 | 89 | 90 |
| Phaeosphaeria nodorum SN15 | 91 | 92 |
| Pichia guilliermondii ATCC 6260 | 95 | 96 |
| Debaryomyces hansenii CBS767 | 97 | 98 |
| Lodderomyces elongisporus NRRL | 99 | 100 |
| YB-4239 | | |
| Vanderwaltozyma polyspora DSM | 109 | 110 |
| 70294 | | |
| Ashbya gossypii ATCC 10895 | 115 | 116 |
| Laccaria bicolor S238N-H82 | 117 | 118 |
| Coprinopsis cinerea okayama7#130 | 119 | 120 |
| Cryptococcus neoformans var. | 121 | 122 |
| neoformans JEC21 | | |
| Ustilago maydis 521 | 123 | 124 |
| Malassezia globosa CBS 7966 | 125 | 126 |
| Aspergillus clavatus NRRL 1 | 127 | 128 |
| Neosartorya fischeri NRRL 181 | 129 | 130 |
| (Putative) | | |
| Aspergillus oryzae | 131 | 132 |
| Aspergillus niger (An18g04160) | 133 | 134 |
| Aspergillus terreus NIH2624 | 135 | 136 |
| Coccidioides immitis RS | 137 | 138 |
| (CIMG_04591) | | |
| Paracoccidioides brasiliensis | 139 | 140 |
| Phaeosphaeria nodorum SN15 | 141 | 142 |
| Gibberella zeae PH-1 | 143 | 144 |
| Neurospora crassa OR74A | 145 | 146 |
| Coprinopsis cinerea okayama 7#130 | 147 | 148 |
| Laccaria bicolor S238N-H82 | 149 | 150 |
| Ustilago maydis 521 | 151 | 152 |

TABLE 3
Expression genes

| | SEQ ID NO: | SEQ ID NO: |
|---|---|---|
| Description | Nucleic acid | Peptide |
| Roseburia inulinivorans (RdhtA) | 15 | 43 |
| Roseburia inulinivorans (RdhtB) | 16 | 44 |
| Bacillus subtilis (alsS) | 27 | 28 |
| Vibrio cholerae (KARI) | 35 | 36 |
| Pseudomonas aeruginosa PAO1 | 37 | 38 |
| (KARI) | | |
| Pseudomonas fluorescens PF5 | 39 | 40 |
| (KARI) | | |
| Achromobacter xylosoxidans (sadB) | 41 | 42 |
| B12-independent glycerol dehydratase | 190 | 191 |
| from Clostridium butyricum | | |
| B-12 independent butanediol | 192 | 193 |
| dehydratase reactivase from | | |
| Clostridium butyricum | | |

SEQ ID NO:17 is a synthetic rdhtAB sequence.

SEQ ID NOs:18-21 and 30-33 are primers for PCR, cloning or sequencing analysis used a described in the Examples herein.

SEQ ID NO:22 is a dual terminator sequence.

SEQ ID NO:23 is the Saccharomyces cerevisiae ADH terminator.

SEQ ID NO:24 is the Saccharomyces cerevisiae CYC1 terminator.

SEQ ID NO:25 is the Saccharomyces cerevisiae FBA promoter.

SEQ ID NO:26 is the Saccharomyces cerevisiae GPM promoter.

SEQ ID NO:29 is the pNY13 vector.

SEQ ID NO:34 is the Saccharomyces cerevisiae CUP1 promoter.

SEQ ID NO:173 is the codon optimized coding region for ILV3 DHAD from Kluyveromyces lactis.

TABLE 4
Functionally verified DHADs used for Profile HMM

| | SEQ ID NO: | SEQ ID NO: |
|---|---|---|
| Organism | Nucleic acid | Peptide |
| Nitrosomonas europaea ATCC 19718 | 174 | 175 |
| Synechocystis sp. PCC 6803 | 176 | 177 |
| Streptococcus mutans UA159 | 178 | 179 |
| Streptococcus thermophilus LMG | 180 | 181 |
| 18311 | | |
| Ralstonia metallidurans CH34 | 182 | 183 |
| Ralstonia eutropha JMP134 | 184 | 185 |
| Lactococcus lactis subsp. cremoris | 186 | 187 |
| SK11 | | |
| Flavobacterium johnsoniae UW101 | 188 | 189 |

### DETAILED DESCRIPTION OF THE INVENTION

Disclosed herein is the discovery that introduced Fe—S containing proteins in yeast host cells have high activity levels when expression of endogenous Fe—S containing proteins is inhibited or disrupted. The present invention relates to recombinant yeast cells engineered to provide expression of at least one heterologous protein that is an Fe—S cluster protein, and engineered for reduced expression of at least one endogenous Fe—S cluster protein. In these cells the activity of the heterologous Fe—S cluster protein is improved, such that there is improved production of a product made in a biosynthetic pathway that includes the enzyme activity. Examples of commercially useful products from a pathway including an Fe—S protein include valine, isoleucine, leucine, pantothenic acid, iosbutanol, 2-butanone and 2-butanol.

The following abbreviations and definitions will be used for the interpretation of the specification and the claims.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “Fe—S cluster protein” is a protein that binds an iron-sulfur cluster and requires the binding of the cluster for its activity.

The term “2Fe-2S DHAD” refers to DHAD enzymes requiring a bound [2Fe-2S]²⁺cluster for activity.

The term “Fe—S propanediol dehydratase reactivase” refers to propanediol dehydratase reactivases requiring a bound Fe—S cluster for activity.

The term “isobutanol biosynthetic pathway” refers to an enzyme pathway to produce isobutanol from pyruvate.

The term “2-butanol biosynthetic pathway” refers to an enzyme pathway to produce 2-butanol from pyruvate.

The term “2-butanone biosynthetic pathway” refers to an enzyme pathway to produce 2-butanone from pyruvate.

There term “Dihydroxy-acid dehydratase”, also abbreviated DHAD , will refer to an enzyme that converts 2,3-dihydroxyisovalerate to α-ketoisovalerate.

The term “butanediol dehydratase”, also known as “diol dehydratase” or “propanediol dehydratase” refers to a polypeptide (or polypeptides) having an enzyme activity that catalyzes the conversion of 2,3-butanediol to 2-butanone. Butanediol dehydratases that do not utilize the cofactor adenosyl cobalamin (also known as coenzyme B12, or vitamin B12; although vitamin B12 may refer also to other forms of cobalamin that are not coenzyme B12) are coenzyme B12-independent diol dehydratases that require association with a diol dehydratase reactivase that is a Fe—S cluster protein. Examples of B12-independent diol dehydratases include those from Clostridium glycolicum (Hartmanis et al. (1986) Arch. Biochem. Biophys. 245:144-152), Clostridium butyricum (protein SEQ ID NO:191; coding region SEQ ID NO:190; O'Brien et al. (2004) Biochemistry 43:4635-4645), and Roseburia inulinivorans (coding: SEQ ID NO:15; protein: SEQ ID NO:43; disclosed in co-pending US Patent Pub No. US20090155870.

The term “propanediol dehydratase reactivase” , also known as “diol dehydratase reactivase” or “butanediol dehydratase reactivase” refers to a reactivating factor for diol dehydratase, an enzyme which undergoes suicide inactivation during catalysis. Diol dehydratase reactivases associated with coenzyme B12-independent diol dehydratases may be Fe—S cluster proteins. Examples include those from Clostridium glycolicum (Hartmanis et al. (1986) Arch. Biochem. Biophys. 245:144-152), Clostridium butyricum (protein SEQ ID NO:193; coding region SEQ ID NO:192; O'Brien et al. (2004) Biochemistry 43:4635-4645), and Roseburia inulinivorans (coding: SEQ ID NO:16; protein: SEQ ID NO:44; disclosed in commonly owned and co-pending US Patent Pub No. US20090155870).

The term “reduced expression” as it applies to the expression of a protein in a cell host will include those situations where the activity of the protein is diminished as compared with a wildtype form (as with antisense technology for example) or substantially eliminated as with gene disruption, deletion or inactivation for example.

The term “carbon substrate” or “fermentable carbon substrate” refers to a carbon source capable of being metabolized by host organisms of the present invention and particularly carbon sources selected from the group consisting of monosaccharides, oligosaccharides, polysaccharides, and one-carbon substrates or mixtures thereof.

The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign gene” or “heterologous gene” refers to a gene not normally found in the host organism, but that is introduced into the host organism by gene transfer. “Heterologous gene” includes a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. Also a foreign gene can comprise native genes inserted into a non-native organism, or chimeric genes. A “transgene” is a gene that has been introduced into the genome by a transformation procedure.

As used herein the term “coding region” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide.

As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein the term “codon degeneracy” refers to the nature in the genetic code permitting variation of the nucleotide sequence without effecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

As used herein, an “isolated nucleic acid fragment” or “isolated nucleic acid molecule” will be used interchangeably and will mean a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A nucleic acid fragment is “hybridizable” to another nucleic acid fragment, such as a cDNA, genomic DNA, or RNA molecule, when a single-stranded form of the nucleic acid fragment can anneal to the other nucleic acid fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washes with 2×SSC, 0.1% SDS followed by 0.1×SSC, 0.1% SDS, for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Altschul, S. F., et al., J. Mol. Biol., 215:403-410 (1993)). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches the complete amino acid and nucleotide sequence encoding particular proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

The term “complementary” is used to describe the relationship between nucleotide bases that are capable of hybridizing to one another. For example, with respect to DNA, adenosine is complementary to thymine and cytosine is complementary to guanine.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: N.Y. (1988); 2.) Biocomputinq: Informatics and Genome Projects (Smith, D. W., Ed.) Academic: N.Y. (1993); 3.) Computer Analysis of Sequence Data, Part I (Griffin, A. M., and Griffin, H. G., Eds.) Humania: N.J. (1994); 4.) Sequence Analysis in Molecular Biology (von Heinje, G., Ed.) Academic (1987); and 5.) Sequence Analysis Primer (Gribskov, M. and Devereux, J., Eds.) Stockton: N.Y. (1991).

Preferred methods to determine identity are designed to give the best match between the sequences tested. Methods to determine identity and similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” corresponding to the alignment method labeled Clustal V (described by Higgins and Sharp, CABIOS. 5:151-153 (1989); Higgins, D.G. et al., Comput. Appl. Biosci., 8:189-191 (1992)) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. Additionally the “Clustal W method of alignment” is available and corresponds to the alignment method labeled Clustal W (described by Higgins and Sharp, CABIOS. 5:151-153 (1989); Higgins, D.G. et al., Comput. Appl. Biosci. 8:189-191(1992)) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

It is well understood by one skilled in the art that many levels of sequence identity are useful in identifying polypeptides, from other species, wherein such polypeptides have the same or similar function or activity. Useful examples of percent identities include, but are not limited to: 55%, 60%, 65%, 70%, 75%, 80%, 85%, 90%, or 95%, or any integer percentage from 55% to 100% may be useful in describing the present invention, such as 55%, 56%, 57%, 58%, 59%, 60%, 81%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987). Additional methods used here are in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.).

### Discovery of Improved Fe—S Cluster Protein Activity in Yeast

Proteins that contain a bound iron-sulfur cluster (Fe—S) that is required for their activity may have low activity when used in heterologous expression systems. Formation of Fe—S clusters and their transfer to apo-proteins is a multistep process involving at least several proteins including cysteine desulfurase, a scaffold protein and a chaperone. Thus a heterologous Fe—S protein may not be effectively composed by the endogenous host system. Applicants have discovered a way to increase activity of an Fe—S protein expressed as a heterologous protein in a yeast host cell. Applicants have found that by reducing production of an endogenous Fe—S protein in the yeast host cell, an improvement in activity of an expressed heterologous Fe—S cluster protein can be achieved. Expression in yeast of either heterologous bacterial, fungal or plant 2Fe-2S dihydroxy-acid dehydratase (DHAD) or Fe—S propanediol dehydratase reactivase (RdhtB) was improved when an endogenous gene encoding isopropylmalate dehydratase (LEU1) or an endogenous gene encoding dihydroxy-acid dehydratase (ILV3) was inactivated in the yeast host cells.

In yeast host cells with inactivation of a gene encoding an endogenous Fe—S protein, the activity of the expressed heterologous Fe—S protein may be increased to at least about 1.4 fold of the activity in a yeast host cell with no inactivation of Fe—S protein encoding gene. For example, the Kluyveromyces lactis DHAD had 1.4 fold activity in a LEU1 deletion host as compared to a host without the deletion; the Roseburia inulinivorans RdhtB had 1.7 fold comparative activity in a LEU deletion host as measured by the activated RdhtA protein activity (described below); Saccharomyces cerevisiae DHAD expressed in the cytosol had 1.5 fold comparative activity in a mitochondrial ILV3 deletion host; and Kluyveromyces lactis DHAD expressed in the cytosol had 7.4 fold comparative activity in a mitochondrial ILV3 deletion host.

Yeast Host Cells with Reduced Expression of Endogenous Fe—S Protein

Reduced endogenous Fe—S protein expression may be engineered in any yeast cell that is amenable to genetic manipulation. Examples include yeasts of Saccharomyces, Schizosaccharomyces, Hansenula, Candida, Kluyveromyces, Yarrowia and Pichia. Suitable strains include, but are not limited to, Saccharomyces cerevisiae, Schizosaccharomyces pombe, Kluyveromyces lactis, Kluyveromyces thermotolerans, Candida glabrata, Candida albicans, Pichia stipitis and Yarrowia lipolytica. Particularly suitable is Saccharomyces cerevisiae.

In any of these yeasts, any endogenous Fe—S protein may be a target for reduced expression. Fe—S proteins in yeast that may be targeted for reduced expression include, for example, the following proteins (with encoding gene): aconitase (ACO1), homoaconitase (LYS4), DHAD (ILV3), lipoate synthase (LIPS), biotin synthase (BIO2), ferredoxin maturation (YAH1), NADH ubiquinone oxidoreductase (NDI1), succinate dehydrogenase (SDH2), ubiquinol-cytochrome-c reductase (RIP1), isopropylmalate isomerase (LEU1), sulfite reductase (ECM17), glutamate dehydrogenase (GLT1), ABC protein Rli1 (RLI1), NTPase Nbp35 (NBP35), and hydrogenase-like protein (NARI1). Yeast cells with reduced expression of individual Fe—S proteins may require special conditions for growth such as supplementation of the growth medium with a particular nutrient, as is well known to one skilled in the art. For example, a strain with disruption of LEU1 is supplemented with leucine, a strain with disruption of DHAD is supplemented with leucine, isoleucine, and valine, and a strain with disruption of LYS4 is supplemented with lysine. Some strains with a disruption require no supplementation for growth. Particularly suitable Fe—S proteins that may be targeted for reduced expression include Isopropylmalate isomerase (LEU1), Dihydroxyacid dehydratase (ILV3), Sulfite reductase (ECM17), Glutamate dehydrogenase (GLT1), and Biotin synthase (BIO2). Reduced expression is engineered for at least one endogenous Fe—S protein, and two or more endogenous Fe—S proteins may be reduced.

LEU1 encodes isopropylmalate dehydratase, an enzyme belonging to EC 4.2.1.33 that is involved in branched chain amino acid biosynthesis, specifically synthesis of leucine. Any gene encoding an isopropylmalate dehydratase, which is an enzyme requiring a 4Fe-4S cluster for activity, may be inactivated in a yeast host cell of this disclosure. Examples of yeast LEU1 inactivation target genes and their encoded proteins are those from Saccharomyces cerevisiae (coding SEQ ID NO:1; protein SEQ ID NO:2), Schizosaccharomyces pombe (coding SEQ ID NO:3; protein SEQ ID NO:4), Candida galbrata strain CBS 138 (coding SEQ ID NO:5; protein SEQ ID NO:6), Candida albicans SC5314 (coding SEQ ID NO:7; protein SEQ ID NO:8), Kluyveromyces lactis (coding SEQ ID NO:; protein SEQ ID NO:10), Yarrowia lipolytica (coding SEQ ID NO:11; protein SEQ ID NO:12) and Pichia stipitis (coding SEQ ID NO:13; protein SEQ ID NO:14).

Similarly in any of the yeast hosts described herein, an endogenous ILV3 gene may be inactivated to reduce endogenous Fe—S protein expression. ILV3 encodes mitochondrial DHAD that is involved in branched chain amino acid biosynthesis. Mitochondrial DHAD is encoded by a nuclear gene, and has a mitochondrial targeting signal sequence so that it is transported to and localized in the mitochondrion. Any ILV3 gene may be inactivated in a yeast host cell of this disclosure. Examples of yeast ILV3 inactivation target genes and their encoded proteins are those from Saccharomyces cerevisiae YJM78 (coding SEQ ID NO:111; protein SEQ ID NO:112), Schizosaccharomyces pombe (coding SEQ ID NO:93; protein SEQ ID NO:94), Candida galbrata strain CBS 138 (coding SEQ ID NO:107; protein SEQ ID NO:108), Candida albicans SC5314 (coding SEQ ID NO:101; protein SEQ ID NO:102), Kluyveromyces lactis (coding SEQ ID NO:113; protein SEQ ID NO:114), Yarrowia lipolytica (coding SEQ ID NO:105; protein SEQ ID NO:106) and Pichia stipitis CBS 6054 (coding SEQ ID NO:103; protein SEQ ID NO:104).

Because genes encoding isopropylmalate dehydratases and DHAD enzymes genes are well known, and because of the prevalence of genomic sequencing, additional suitable species of these enzymes can be readily identified by one skilled in the art on the basis of sequence similarity using bioinformatics approaches. Typically BLAST (described above) searching of publicly available databases with known isopropylmalate dehydratase amino acid sequences, such as those provided herein, is used to identify these enzymes and their encoding sequences that may be targeted for inactivation in the present strains. For example, endogenous yeast isopropylmalate dehydratase and DHAD proteins having amino acid sequence identities of at least about 70-75%, 75%-80%, 80-85%, 85%-90%, 90%-95% or 98% A sequence identity to any of the isopropylmalate dehydratase proteins of SEQ ID NOs:2, 4, 6 ,8, 10, 12 and 14 and the DHAD proteins of SEQ ID NOs:94, 102, 104, 106, 108, 112, and 114 may have reduced expression in the present strains. Identities are based on the Clustal W method of alignment using the default parameters of GAP PENALTY=10, GAP LENGTH PENALTY=0.1, and Gonnet 250 series of protein weight matrix.

Additionally, the sequences of LEU1 coding regions and ILV3 provided herein may be used to identify other homologs in nature. For example each of the coding regions described herein may be used to isolate genes encoding homologous proteins. Isolation of homologous genes using sequence-dependent protocols is well known in the art. Examples of sequence-dependent protocols include, but are not limited to: 1.) methods of nucleic acid hybridization; 2.) methods of DNA and RNA amplification, as exemplified by various uses of nucleic acid amplification technologies [e.g., polymerase chain reaction (PCR), Mullis et al., U.S. Pat. No. 4,683,202; ligase chain reaction (LCR), Tabor, S. et al., Proc. Acad. Sci. USA 82:1074 (1985); or strand displacement amplification (SDA), Walker, et al., Proc. Natl. Acad. Sci. U.S.A., 89:392 (1992)]; and 3.) methods of library construction and screening by complementation.

For example, genes encoding similar proteins or polypeptides to the isopropylmalate dehydratase and DHAD encoding genes provided herein could be isolated directly by using all or a portion of the instant nucleic acid fragments as DNA hybridization probes to screen libraries from any desired organism using methodology well known to those skilled in the art. Specific oligonucleotide probes based upon the disclosed nucleic acid sequences can be designed and synthesized by methods known in the art (Maniatis, supra). Moreover, the entire sequences can be used directly to synthesize DNA probes by methods known to the skilled artisan (e.g., random primers DNA labeling, nick translation or end-labeling techniques), or RNA probes using available in vitro transcription systems. In addition, specific primers can be designed and used to amplify a part of (or full-length of) the instant sequences. The resulting amplification products can be labeled directly during amplification reactions or labeled after amplification reactions, and used as probes to isolate full-length DNA fragments by hybridization under conditions of appropriate stringency.

Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The use of oligonucleotides as specific hybridization probes in the Diagnosis of Genetic Disorders”, in Human Genetic Diseases: A Practical Approach, K. E. Davis Ed., (1986) pp 33-50, IRL: Herndon, Va.; and Rychlik, W., In Methods in Molecular Biology, White, B. A. Ed., (1993) Vol. 15, pp 31-39, PCR Protocols: Current Methods and Applications. Humania: Totowa, N.J.).

Generally two short segments of the described sequences may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the described nucleic acid fragments, and the sequence of the other primer takes advantage of the presence of the polyadenylic acid tracts to the 3′ end of the mRNA precursor encoding microbial genes.

Alternatively, the second primer sequence may be based upon sequences derived from the cloning vector. For example, the skilled artisan can follow the RACE protocol (Frohman et al., PNAS USA 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (e.g., BRL, Gaithersburg, Md.), specific 3′ or 5′ cDNA fragments can be isolated (Ohara et al., PNAS USA 86:5673 (1989); Loh et al., Science 243:217 (1989)).

Alternatively, the provided isopropylmalate dehydratase and DHAD encoding sequences can be employed as hybridization reagents for the identification of homologs. The basic components of a nucleic acid hybridization test include a probe, a sample suspected of containing the gene or gene fragment of interest, and a specific hybridization method. Probes are typically single-stranded nucleic acid sequences that are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions that will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration, the shorter the hybridization incubation time needed. Optionally, a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen, Nucl. Acids Res. 19:5143-5151 (1991)). Suitable chaotropic agents include guanidinium chloride, guanidinium thiocyanate, sodium thiocyanate, lithium tetrachloroacetate, sodium perchlorate, rubidium tetrachloroacetate, potassium iodide and cesium trifluoroacetate, among others. Typically, the chaotropic agent will be present at a final concentration of about 3 M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers (e.g., sodium citrate, Tris-HCl, PIPES or HEPES (pH range about 6-9)), about 0.05 to 0.2% detergent (e.g., sodium dodecylsulfate), or between 0.5-20 mM EDTA, FICOLL (Pharmacia Inc.) (about 300-500 kdal), polyvinylpyrrolidone (about 250-500 kdal) and serum albumin. Also included in the typical hybridization solution will be unlabeled carrier nucleic acids from about 0.1 to 5 mg/mL, fragmented nucleic DNA (e.g., calf thymus or salmon sperm DNA, or yeast RNA), and optionally from about 0.5 to 2% wt/vol glycine. Other additives may also be included, such as volume exclusion agents that include a variety of polar water-soluble or swellable agents (e.g., polyethylene glycol), anionic polymers (e.g., polyacrylate or polymethylacrylate) and anionic saccharidic polymers (e.g., dextran sulfate).

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

Protein and nucleic acid encoding sequences for any of the other Fe—S proteins that may be targeted for reduced activity in a yeast cell of the invention may be identified using bioinformatics and other methods well known to one skilled in the art. For example, aconitase sequences are identified by keyword searching in bioinformatics databases. Several sequences identified by this method are those from Saccharomyces cerevisiae (coding SEQ ID NO:153; protein SEQ ID NO:154), Schizosaccharomyces pombe on chromosome II (coding SEQ ID NO:155; protein SEQ ID NO:156), Schizosaccharomyces pombe on chromosome I (coding SEQ ID NO:157; protein SEQ ID NO:158), Kluyveromyces lactis (coding SEQ ID NO:15; protein SEQ ID NO:160), Candida albicans SC5314 (coding SEQ ID NO:161; protein SEQ ID NO:162), Yarrowia lipolytica (coding SEQ ID NO:163; protein SEQ ID NO:164), Pichia stipitis CBS 6054 (coding SEQ ID NO:165; protein SEQ ID NO:166), Candida galbrata CBS 138 chromosome F (coding SEQ ID NO:167; protein SEQ ID NO:168), Candida galbrata CBS 138 chromosome D (coding SEQ ID NO:169; protein SEQ ID NO:170), and Candida galbrata CBS 138 chromosome K (coding SEQ ID NO:171; protein SEQ ID NO:172).

Genes encoding Fe—S proteins, for example LEU1, ILV3, or ACO1 may be disrupted in any yeast cell using genetic modification. Many methods for genetic modification of target genes are known to one skilled in the art and may be used to create the present yeast strains. Modifications that may be used to reduce or eliminate expression of a target protein are disruptions that include, but are not limited to, deletion of the entire gene or a portion of the gene, inserting a DNA fragment into the gene (in either the promoter or coding region) so that the protein is not expressed or expressed at lower levels, introducing a mutation into the coding region which adds a stop codon or frame shift such that a functional protein is not expressed, and introducing one or more mutations into the coding region to alter amino acids so that a non-functional or a less enzymatically active protein is expressed. In addition, expression of a gene may be blocked by expression of an antisense RNA or an interfering RNA, and constructs may be introduced that result in cosuppression. In addition, the synthesis or stability of the transcript may be lessened by mutation. Similarly the efficiency by which a protein is translated from mRNA may be modulated by mutation. All of these methods may be readily practiced by one skilled in the art making use of the known or identified coding sequences such as LEU1 or ILV3.

DNA sequences surrounding a LEU1, ILV3, or ACO1 coding sequence are also useful in some modification procedures and are available for yeasts such as for Saccharomycse cerevisiae in the complete genome sequence coordinated by Genome Project ID9518 of Genome Projects coordinated by NCBI (National Center for Biotechnology Information) with identifying GOPID 13838. Additional examples of yeast genomic sequences include that of Yarrowia lipolytica, GOPIC 13837, and of Candida albicans, which is included in GPID 10771,10701 and 16373. Additional genomes have been completely sequenced and annotated and are publicly available for the following yeast strains Candida glabrata CBS 138, Kluyveromyces lactis NRRL Y-1140, Pichia stipitis CBS 6054, and Schizosaccharomyces pombe 972h-.

In particular, DNA sequences surrounding a target coding sequence, such as LEU1 or ILV3, are useful for modification methods using homologous recombination. For example, in this method flanking sequences are placed bounding a selectable marker gene to mediate homologous recombination whereby the marker gene replaces the target gene. Also partial target gene sequences and flanking sequences bounding a selectable marker gene may be used to mediate homologous recombination whereby the marker gene replaces a portion of the target gene. In addition, the selectable marker may be bounded by site-specific recombination sites, so that following expression of the corresponding site-specific recombinase, the resistance gene is excised from the target gene without reactivating the latter. The site-specific recombination leaves behind a recombination site which disrupts expression of the target gene encoded protein. The homologous recombination vector may be constructed to also leave a deletion in the target gene following excision of the selectable marker, as is well known to one skilled in the art.

Deletions may be made using mitotic recombination as described in Wach et al. ((1994) Yeast 10:1793-1808). This method involves preparing a DNA fragment that contains a selectable marker between genomic regions that may be as short as 20 bp, and which bound a target DNA sequence. This DNA fragment can be prepared by PCR amplification of the selectable marker gene using as primers oligonucleotides that hybridize to the ends of the marker gene and that include the genomic regions that can recombine with the yeast genome. The linear DNA fragment can be efficiently transformed into yeast and recombined into the genome resulting in gene replacement including with deletion of the target DNA sequence (as described in Methods in Enzymology, v194, pp 281-301 (1991)).

Moreover, promoter replacement methods may be used to exchange the endogenous transcriptional control elements allowing another means to modulate expression such as described in Mnaimneh et al. ((2004) Cell 118(1):31-44) and in Example 12 herein.

In addition, a target gene in any yeast cell may be disrupted using random mutagenesis, which is followed by screening to identify strains with reduced target gene encided activity. Using this type of method, the DNA sequence of for example the LEU1, ILV3, or any other region of the genome affecting expression of a target Fe—S protein, need not be known.

Methods for creating genetic mutations are common and well known in the art and may be applied to the exercise of creating mutants. Commonly used random genetic modification methods (reviewed in Methods in Yeast Genetics, 2005, Cold Spring Harbor Laboratory Press, Cold Spring Harbor, N.Y.) include spontaneous mutagenesis, mutagenesis caused by mutator genes, chemical mutagenesis, irradiation with UV or X-rays, or transposon mutagenesis.

Chemical mutagenesis of yeast commonly involves treatment of yeast cells with one of the following DNA mutagens: ethyl methanesulfonate (EMS), nitrous acid, diethyl sulfate, or N-methyl-N′-nitro-N-nitroso-guanidine (MNNG).These methods of mutagenesis have been reviewed in Spencer et al (Mutagenesis in Yeast, 1996, Yeast Protocols: Methods in Cell and Molecular Biology. Humana Press, Totowa, N.J.). Chemical mutagenesis with EMS may be performed as described in Methods in Yeast Genetics, 2005, Cold Spring Harbor Laboratory Press, Cold Spring Harbor, N.Y. Irradiation with ultraviolet (UV) light or X-rays can also be used to produce random mutagenesis in yeast cells. The primary effect of mutagenesis by UV irradiation is the formation of pyrimidine dimers which disrupt the fidelity of DNA replication. Protocols for UV-mutagenesis of yeast can be found in Spencer et al (Mutagenesis in Yeast, 1996, Yeast Protocols: Methods in Cell and Molecular Biology. Humana Press, Totowa, N.J.). Introduction of a mutator phenotype can also be used to generate random chromosomal mutations in yeast. Common mutator phenotypes can be obtained through disruption of one or more of the following genes: PMS1, MAG1, RAD18 or RAD51. Restoration of the non-mutator phenotype can be easily obtained by insertion of the wildtype allele. Collections of modified cells produced from any of these or other known random mutagenesis processes may be screened for reduced Fe—S protein activity.

### Heterologous Fe—S Proteins

Any bacterial, fungal or plant 2Fe-2S cluster dihydroxy-acid dehydratase (DHAD) and any Fe—S propanediol dehydratase reactivase may be expressed as a heterologous protein in a yeast host cell engineered as disclosed herein for reduced endogenous Fe—S cluster protein expression, and increased activity may be obtained. A heterologous protein includes one that is expressed in a manner differently from expression of a corresponding endogenous protein. For example in yeast, endogenous DHAD is encoded by ILV3 in the nucleus and the expressed DHAD protein has a mitochondrial targeting signal sequence such that the protein is localized in the mitochondrion. An Fe—S cluster is added to the DHAD protein in the mitochondrion for its activity in branched chain amino acid biosynthesis. It is desirable to express DHAD activity in the cytosol for participation in biosynthetic pathways that are localized in the cytosol. Cytosolic expression of DHAD in yeast is heterologous expression since the native protein is localized in the mitochondrion. For example, heterologous expression of the Saccharomyces cerevisiae DHAD in S. cerevisiae is obtained by expressing the S. cerevisiae DHAD coding region with the mitochondrial targeting signal removed, such that the protein remains in the cytosol. 2Fe-2S DHADs that may be used in the present disclosure include those from fungi and plants. Representative bacterial, fungal or plant 2Fe-2S DHADs are listed in Tables 1, 2, and 4. Bacterial, fungal or plant 2Fe-2S DHADs with amino acid sequence identities of 95% or greater were removed from the analysis providing this list for simplification. However, any sequences with 95% or greater amino acid identities to any of the sequences listed in Tables 1, 2, and 4 are useful in the present invention. The analysis used to obtain 2Fe-2S DHADs is described in commonly owned and co-pending U.S. Patent Application No. 61/100,792, which is herein incorporated by reference. The analysis is as follows: Therein a Profile Hidden Markov Model (HMM) was prepared based on amino acid sequences of eight functionally verified DHADs. These DHADs are from Nitrosomonas europaea (DNA SEQ ID NO:174; Protein SEQ ID NO:175), Synechocystis sp. PCC6803 (DNA SEQ ID:176; Protein SEQ ID NO:177), Streptococcus mutans (DNA SEQ ID NO:178; Protein SEQ ID NO:179), Streptococcus thermophilus (DNA SEQ ID NO:180; protein SEQ ID No:181), Ralstonia metallidurans (DNA SEQ ID NO:182; protein SEQ ID NO:183), Ralstonia eutropha (DNA SEQ ID NO:184; protein SEQ ID NO:185), and Lactococcus lactis (DNA SEQ ID NO:186; protein SEQ ID NO:187). In addition the DHAD from Flavobacterium johnsoniae (DNA SEQ ID NO:188; protein SEQ ID NO:189) was found to have dihydroxy-acid dehydratase activity when expressed in E. coli and was used in making the Profile. The Profile HMM is prepared using the HMMER software package (The theory behind profile HMMs is described in R. Durbin, S. Eddy, A. Krogh, and G. Mitchison, Biological sequence analysis: probabilistic models of proteins and nucleic acids, Cambridge University Press, 1998; Krogh et al., 1994; J. Mol. Biol. 235:1501-1531), following the user guide which is available from HMMER (Janelia Farm Research Campus, Ashburn, Va.). The output of the HMMER software program is a Profile Hidden Markov Model (HMM) that characterizes the input sequences, given in Table 9.

Any protein that matches the Profile HMM with an E value of <10⁻⁵ is a DHAD related protein, which includes 4Fe-4S DHADs, 2Fe-2S DHADs, arabonate dehydratases, and phosphogluconate dehydratases. Sequences matching the Profile HMM are then analyzed for the presence of the three conserved cysteines, corresponding to positions 56, 129, and 201 in the Streptococcus mutans DHAD. The presence of all three conserved cysteines is characteristic of proteins having a [2Fe-2S] ²⁺cluster. Proteins having the three conserved cysteines include arabonate dehydratases and 2Fe-2S DHADs. The 2Fe-2S DHADs may be distinguished from the arabonate dehydratases by analyzing for signature conserved amino acids found to be present in the 2Fe-2S DHADs or in the arabonate dehydratases at positions corresponding to the following positions in the Streptococcus mutans DHAD amino acid sequence. These signature amino acids are in 2Fe-2S DHADs or in arabonate dehydratases, respectively, at the following positions (with greater than 90% occurance): 88 asparagine vs glutamic acid; 113 not conserved vs glutamic acid; 142 arginine or asparagine vs not conserved; 165: not conserved vs glycine; 208 asparagine vs not conserved; 454 leucine vs not conserved; 477 phenylalanine or tyrosine vs not conserved; and 487 glycine vs not conserved.

The proteins identified by this process that have a fungal or plant origin, such as SEQ ID NOs:46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150 and 152 may be used in the present invention, as well as any protein with amino acid identity of at least about 95%, 96%, 97%, 98%, or or (:)/o to any of these sequences. Particularly suitable is the DHAD from Kluyveromyces lactis (SEQ ID NO:114) and DHADs with at least about 90% amino acid sequence identity to SEQ ID NO:114 using the Clustal W method of alignment using the default parameters of GAP PENALTY=10, GAP LENGTH PENALTY=0.1, and Gonnet 250 series of protein weight matrix over the full length of the protein sequence.

The DHAD proteins of SEQ ID NOs:175, 177, 179, 181, 183, 185, 187, and 189 may also be used in the present invention, as well as any protein with amino acid identity of at least about 95%, 96%, 97%, 98%, or 99% to any of these sequences using the Clustal W method of alignment using the default parameters of GAP PENALTY=10, GAP LENGTH PENALTY=0.1, and Gonnet 250 series of protein weight matrix over the full length of the protein sequence.

In addition, fungal or plant 2Fe-2S DHADs that may be used in the present invention may be identified by their position in a fungal or plant 2Fe-2S DHAD branch of a phylogenetic tree of DHAD related proteins. In addition, 2Fe-2S DHADs that may be used may be identified using sequence comparisons with any of the fungal or plant 2Fe-2S DHADs whose sequences are provided herein, where sequence identity may be at least about 80%-85%, 85%-90%, 90%-95% or 95%-99%.

Additionally, the sequences of fungal or plant 2Fe-2S DHADs provided herein may be used to identify other homologs in nature. For example each of the DHAD encoding nucleic acid fragments given herein as SEQ ID NOs:45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 11, 121, 123, 125, 127, 129, 131, 133, 135, 137, 13, 141, 143, 145, 147, 149 and 151 may be used to isolate genes encoding homologous proteins as described above for the LEU1 coding region.

The coenzyme B12-independent propanediol deydratase reactivase of Roseburia inulinivorans is a protein requiring an Fe—S cluster for activity. This protein, RdhtB, is disclosed in co-pending US Patent Pub No. US20090155870, which is herein incorporated by reference. RdhtB reactivates a coenzyme B12-independent propanediol deydratase of Roseburia inulinivorans, which is named RdhtA and is also disclosed in commonly owned and co-pending US Patent Pub No. US20090155870. The activity of RdhtB may be assessed by assaying the activity of RdhtA, since RdhtB is required for RdhtA activity. Activity of RdhtB, and therefore of RdhtA, is improved by expressing in a yeast host with reduced endogenous Fe—S protein expression disclosed herein. Heterologous expression of any coenzyme B12-independent propanediol deydratase reactivase that requires an Fe—S cluster for activity may be improved in a yeast strain having reduced endogenous Fe—S protein expression. A coenzyme B12-independent propanediol deydratase reactivase may be readily identified by one skilled in the art by assessing propanediol deydratase activity of the associated propanediol deydratase enzyme in the presence or absence of coenzyme B12. An example is a diol dehydratase reactivase of Clostridium butyricum (coding region SEQ ID NO:192; protein SEQ ID NO:193).

Other coenzyme B12-independent propanediol deydratase reactivases that may be used may be identified through bioinformatics analysis of sequences as compared to that of RdhtB SEQ ID NO:44 by one skilled in the art. Proteins with coenzyme B12-independent propanediol deydratase reactivase activity and sequence identity to SEQ ID NO:44 of at least about 80%-85%, 85%-90%, 90%-95% or 95%-99% may be used. Particularly suitable are those that are at least about 90% identical to the amino acid sequence as set forth in SEQ ID NO:44 using the Clustal W method of alignment using the default parameters of GAP PENALTY=10, GAP LENGTH PENALTY=0.1, and Gonnet 250 series of protein weight matrix over the full length of the protein sequence.

In addition, other coenzyme B12-independent propanediol deydratase reactivase homologs that may be used may be identified using the RdhtB coding region (SEQ ID NO:16) by methods as described above for the LEU1 coding region.

### Expression of Heterologous Fe—S Proteins

Expression is achieved by transforming with a sequence encoding an Fe—S protein. The coding region to be expressed may be codon optimized for the target host cell, as well known to one skilled in the art. Methods for gene expression in yeast are known in the art (see for example Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). Expression of genes in yeast typically requires a promoter, operably linked to a coding region of interest, and a transcriptional terminator. A number of yeast promoters can be used in constructing expression cassettes for genes in yeast, including, but not limited to promoters derived from the following genes: CYC1, HIS3, GAL1, GAL10, ADH1, PGK, PHO5, GAPDH, ADC1, TRP1, URA3, LEU2, ENO, TPI, CUP1, FBA, GPD, GPM, and AOX1. Suitable transcriptional terminators include, but are not limited to FBAt, GPDt, GPMt, ERG10t, GAL1t, CYC1, and ADH1.

Suitable promoters, transcriptional terminators, and coding regions may be cloned into E. coli-yeast shuttle vectors, and transformed into yeast cells. These vectors allow strain propagation in both E. coli and yeast strains. Typically the vector used contains a selectable marker and sequences allowing autonomous replication or chromosomal integration in the desired host. Typically used plasmids in yeast are shuttle vectors pRS423, pRS424, pRS425, and pRS426 (American Type Culture Collection, Rockville, Md.), which contain an E. coli replication origin (e.g., pMB1), a yeast 2μorigin of replication, and a marker for nutritional selection. The selection markers for these four vectors are His3 (vector pRS423), Trp1 (vector pRS424), Leu2 (vector pRS425) and Ura3 (vector pRS426). Construction of expression vectors with a chimeric gene encoding the described Fe—S protein coding regions may be performed by either standard molecular cloning techniques in E. coli or by the gap repair recombination method in yeast.

The gap repair cloning approach takes advantage of the highly efficient homologous recombination in yeast. Typically, a yeast vector DNA is digested (e.g., in its multiple cloning site) to create a “gap” in its sequence. A number of insert DNAs of interest are generated that contain a ≧21 bp sequence at both the 5′ and the 3′ ends that sequentially overlap with each other, and with the 5′ and 3′ terminus of the vector DNA. For example, to construct a yeast expression vector for “Gene X”, a yeast promoter and a yeast terminator are selected for the expression cassette. The promoter and terminator are amplified from the yeast genomic DNA, and Gene X is either PCR amplified from its source organism or obtained from a cloning vector comprising Gene X sequence. There is at least a 21 bp overlapping sequence between the 5′ end of the linearized vector and the promoter sequence, between the promoter and Gene X, between Gene X and the terminator sequence, and between the terminator and the 3′ end of the linearized vector. The “gapped” vector and the insert DNAs are then co-transformed into a yeast strain and plated on the medium containing the appropriate compound mixtures that allow complementation of the nutritional selection markers on the plasmids. The presence of correct insert combinations can be confirmed by PCR mapping using plasmid DNA prepared from the selected cells. The plasmid DNA isolated from yeast (usually low in concentration) can then be transformed into an E. coli strain, e.g. TOP10, followed by mini preps and restriction mapping to further verify the plasmid construct. Finally the construct can be verified by sequence analysis.

Like the gap repair technique, integration into the yeast genome also takes advantage of the homologous recombination system in yeast. Typically, a cassette containing a coding region plus control elements (promoter and terminator) and auxotrophic marker is PCR-amplified with a high-fidelity DNA polymerase using primers that hybridize to the cassette and contain 40-70 base pairs of sequence homology to the regions 5′ and 3′ of the genomic area where insertion is desired. The PCR product is then transformed into yeast and plated on medium containing the appropriate compound mixtures that allow selection for the integrated auxotrophic marker. For example, to integrate “Gene X” into chromosomal location “Y”, the promoter-coding regionX-terminator construct is PCR amplified from a plasmid DNA construct and joined to an autotrophic marker (such as URA3) by either SOE PCR or by common restriction digests and cloning. The full cassette, containing the promoter-coding regionX-terminator-URA3 region, is PCR amplified with primer sequences that contain 40-70 bp of homology to the regions 5′ and 3′ of location “Y” on the yeast chromosome. The PCR product is transformed into yeast and selected on growth media lacking uracil. Transformants can be verified either by colony PCR or by direct sequencing of chromosomal DNA.

Any coding regions expressed in the present yeast cells may be codon optimized for expression in the specific host yeast cell being engineered as is well known to one skilled in the art. For example, for expression of the K. lactis and P. stipitis ILV3 coding regions in S. cerevisiae, each was codon optimized for S. cerevisiae expression in Example 1 herein.

Product Biosynthesis with Improved Heterologous Fe—S Protein Activity

Production of any product that has an Fe—S protein contributing to its biosynthetic pathway may benefit from the improved activity disclosed herein of a heterologous expressed Fe—S protein in a yeast host with reduced endogenous Fe—S protein expression. For example, DHAD provides a step in pathways for biosynthesis of isobutanol, and RdhtB contributes to a biosynthetic pathway to produce 2-butanone or 2-butanol.

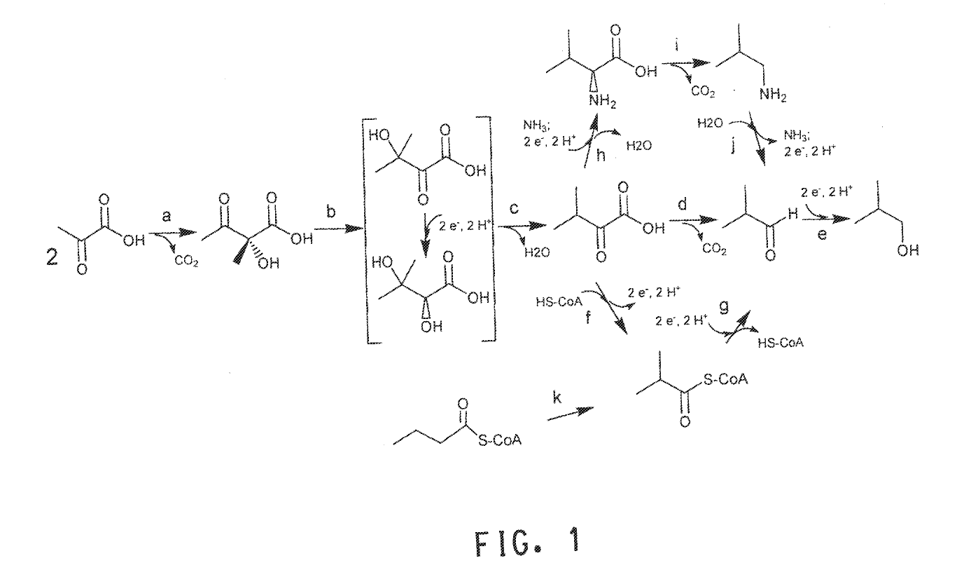

Biosynthetic pathways including a step performed by DHAD for synthesis of isobutanol are disclosed in commonly owned and co-pending US Patent Application publication US 20070092957 A1, which is herein incorporated by reference. A diagram of the disclosed isobutanol biosynthetic pathways is provided in FIG. 1. Production of isobutanol in a strain disclosed herein benefits from increased DHAD activity. As described in US Patent Pub No. US20070092957 A1, steps in an example isobutanol biosynthetic pathway include conversion of:

pyruvate to acetolactate (FIG. 1 pathway step a) as catalyzed for example by acetolactate synthase;

acetolactate to 2,3-dihydroxyisovalerate (FIG. 1 pathway step b) as catalyzed for example by acetohydroxy acid isomeroreductase;

2,3-dihydroxyisovalerate to α-ketoisovalerate (FIG. 1 pathway step c) as catalyzed for example by acetohydroxy acid dehydratase also called DHAD;

α-ketoisovalerate to isobutyraldehyde (FIG. 1 pathway step d) as catalyzed for example by branched-chain α-keto acid decarboxylase; and

isobutyraldehyde to isobutanol (FIG. 1 pathway step e) as catalyzed for example by branched-chain alcohol dehydrogenase.

The substrate to product conversions, and enzymes involved in these reactions, for steps f, g, h, I, j, and k of alternative pathways are described in US 20070092957 A1.

Genes that may be used for expression of the enzymes for the isobutanol pathways are described in US 20070092957 A1, and additional genes that may be used can be identified by one skilled in the art through bioinformatics or experimentally as described above. The preferred use in all three pathways of ketol-acid reductoisomerase (KARI) enzymes with particularly high activities are disclosed in commonly owned and co-pending US Patent Pub No. US20080261230. Examples of high activity KARIs disclosed therein are those from Vibrio cholerae (DNA: SEQ ID NO:35; protein SEQ ID NO:36), Pseudomonas aeruginosa PAO1, (DNA: SEQ ID NO:37; protein SEQ ID NO:38), and Pseudomonas fluorescens PF5 (DNA: SEQ ID NO:39; protein SEQ ID NO:40).

Additionally described in US 20070092957 A1 are construction of chimeric genes and genetic engineering of yeast, exemplified by Saccharomyces cerevisiae, for isobutanol production using the disclosed biosynthetic pathways.

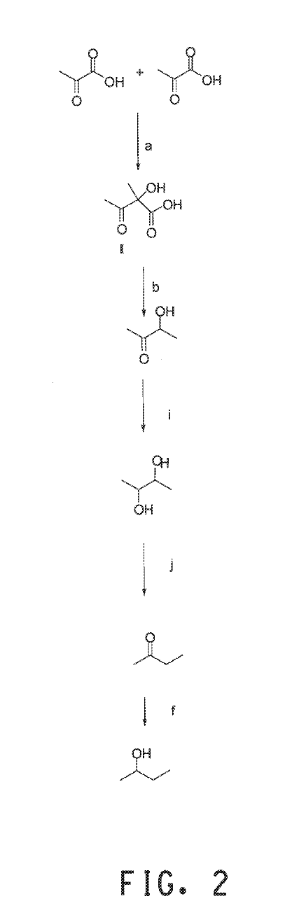

A biosynthetic pathway including propanediol dehydratase for synthesis of 2-butanone and 2-butanol is disclosed in commonly owned and co-pending US Patent Pub No. US20070292927A1, which is herein incorporated by reference. A diagram of the disclosed 2-butanone and 2-butanol biosynthetic pathway is provided in FIG. 2. 2-Butanone is the product made when the last depicted step of converting 2-butanone to 2-butanol is omitted. Production of 2-butanone or 2-butanol in a strain disclosed herein benefits from increased coenzyme B12-independent propanediol dehydratase reactivase activity. As described in US Patent Pub No. US20070292927A1, steps in the disclosed biosynthetic pathway include conversion of:

pyruvate to acetolactate (FIG. 2 step a) as catalyzed for example by acetolactate synthase;

acetolactate to acetoin (FIG. 2 step b) as catalyzed for example by acetolactate decarboxylase;

acetoin to 2,3-butanediol (FIG. 2 step i) as catalyzed for example by butanediol dehydrogenase;

2,3-butanediol to 2-butanone (FIG. 2 step j) as catalyzed for example by diol dehydratase glycerol dehydratase, or propanediol dehydratase; and

2-butanone to 2-butanol (FIG. 2 step f) as catalyzed for example by butanol dehydrogenase.

Genes that may be used for expression of these enzymes are described in US Patent Pub No. US20070292927A1. The use in this pathway in yeast of the butanediol dehydratase from Roseburia inulinivorans, RdhtA, (protein SEQ ID NO:43, coding region SEQ ID NO:15) is disclosed in commonly owed and co-pending US Patent Pub No. US20090155870. This enzyme is used in conjunction with the butanediol dehydratase reactivase from Roseburia inulinivorans, RdhtB, (protein SEQ ID NO:44, coding region SEQ ID NO:16). This butanediol dehydratase is desired in many hosts because it does not require coenzyme B₁₂.

Additionally described in US Patent Pub No. US20090155870are construction of chimeric genes and genetic engineering of yeast for 2-butanol production using the US 20070292927A1 disclosed biosynthetic pathway.

### Fermentation Media

Yeasts disclosed herein may be grown in fermentation media for production of a product having an Fe—S protein as part of the biosynthetic pathway. Fermentation media must contain suitable carbon substrates. Suitable substrates may include but are not limited to monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally the carbon substrate may also be one-carbon substrates such as carbon dioxide, or methanol for which metabolic conversion into key biochemical intermediates has been demonstrated. In addition to one and two carbon substrates methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1 Compd., [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the above mentioned carbon substrates and mixtures thereof are suitable in the present invention, preferred carbon substrates are glucose, fructose, and sucrose.

In addition to an appropriate carbon source, fermentation media must contain suitable minerals, salts, cofactors, buffers and other components, known to those skilled in the art, suitable for the growth of the cultures and promotion of the enzymatic pathway necessary for production of the desired product.

### Culture Conditions

Typically cells are grown at a temperature in the range of about 20° C. to about 37° C. in an appropriate medium. Suitable growth media in the present invention are common commercially prepared media such as broth that includes yeast nitrogen base, ammonium sulfate, and dextrose as the carbon/energy source) or YPD Medium, a blend of peptone, yeast extract, and dextrose in optimal proportions for growing most Saccharomyces cerevisiae strains. Other defined or synthetic growth media may also be used and the appropriate medium for growth of the particular microorganism will be known by one skilled in the art of microbiology or fermentation science.

Suitable pH ranges for the fermentation are between pH 3.0 to pH 7.5, where pH 4.5.0 to pH 6.5 is preferred as the initial condition.

Fermentations may be performed under aerobic or anaerobic conditions, where anaerobic or microaerobic conditions are preferred.

The amount of butanol produced in the fermentation medium can be determined using a number of methods known in the art, for example, high performance liquid chromatography (HPLC) or gas chromatography (GC).

### Industrial Batch and Continuous Fermentations

The present process employs a batch method of fermentation. A classical batch fermentation is a closed system where the composition of the medium is set at the beginning of the fermentation and not subject to artificial alterations during the fermentation. Thus, at the beginning of the fermentation the medium is inoculated with the desired organism or organisms, and fermentation is permitted to occur without adding anything to the system. Typically, however, a “batch” fermentation is batch with respect to the addition of carbon source and attempts are often made at controlling factors such as pH and oxygen concentration. In batch systems the metabolite and biomass compositions of the system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of end product or intermediate.

A variation on the standard batch system is the Fed-Batch system. Fed-Batch fermentation processes are also suitable in the present invention and comprise a typical batch system with the exception that the substrate is added in increments as the fermentation progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

Although the present invention is performed in batch mode it is contemplated that the method would be adaptable to continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

The present invention may be practiced using either batch, fed-batch or continuous processes and known modes of fermentation are suitable. Additionally, it is contemplated that cells may be immobilized on a substrate as whole cell catalysts and subjected to fermentation conditions for 1-butanol production.

Methods for Butanol Isolation from the Fermentation Medium

Bioproduced butanol may be isolated from the fermentation medium using methods known in the art. For example, solids may be removed from the fermentation medium by centrifugation, filtration, decantation, or the like. Then, the butanol may be isolated from the fermentation medium, which has been treated to remove solids as described above, using methods such as distillation, liquid-liquid extraction, or membrane-based separation. Because butanol forms a low boiling point, azeotropic mixture with water, distillation can only be used to separate the mixture up to its azeotropic composition. Distillation may be used in combination with another separation method to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer (see for example Doherty and Malone, Conceptual Design of Distillation Systems, McGraw Hill, New York, 2001).

The butanol-water mixture forms a heterogeneous azeotrope so that distillation may be used in combination with decantation to isolate and purify the butanol. In this method, the butanol containing fermentation broth is distilled to near the azeotropic composition. Then, the azeotropic mixture is condensed, and the butanol is separated from the fermentation medium by decantation. The decanted aqueous phase may be returned to the first distillation column as reflux. The butanol-rich decanted organic phase may be further purified by distillation in a second distillation column.

The butanol may also be isolated from the fermentation medium using liquid-liquid extraction in combination with distillation. In this method, the butanol is extracted from the fermentation broth using liquid-liquid extraction with a suitable solvent. The butanol-containing organic phase is then distilled to separate the butanol from the solvent.

Distillation in combination with adsorption may also be used to isolate butanol from the fermentation medium. In this method, the fermentation broth containing the butanol is distilled to near the azeotropic composition and then the remaining water is removed by use of an adsorbent, such as molecular sieves (Aden et al. Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the butanol from the fermentation medium. In this method, the fermentation broth containing the butanol is distilled to near the azeotropic composition, and then the remaining water is removed by pervaporation through a hydrophilic membrane (Guo et al., J. Membr. Sci. 245, 199-210 (2004)).

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, N.Y. (1989) (Maniatis) and by T. J. Silhavy, M. L. Bennan, and L. W. Enquist, Experiments with Gene Fusions, Cold Spring Harbor Laboratory Press, Cold Spring Harbor, N.Y. (1984) and by Ausubel, F. M. et al., Current Protocols in Molecular Biology, pub. by Greene Publishing Assoc. and Wiley-Interscience (1987), and by Methods in Yeast Genetics, 2005, Cold Spring Harbor Laboratory Press, Cold Spring Harbor, N.Y.

Materials and methods suitable for the maintenance and growth of bacterial cultures are well known in the art. Techniques suitable for use in the following Examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, DC. (1994)) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of microbial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.) unless otherwise specified. Microbial strains were obtained from The American Type Culture Collection (ATCC), Manassas, Va., unless otherwise noted. All the oligonucleotide primers were synthesized by Sigma-Genosys (Woodlands, Tex.) or Integrated DNA Technologies (Coralsville, Iowa).

Synthetic complete medium is described in Amberg, Burke and Strathern, 2005, Methods in Yeast Genetics, Cold Spring Harbor Laboratory Press, Cold Spring Harbor, N.Y.

### HPLC

Analysis for fermentation by-product composition is well known to those skilled in the art. For example, one high performance liquid chromatography (HPLC) method utilizes a Shodex SH-1011 column with a Shodex SH-G guard column (both available from Waters Corporation, Milford, Mass.), with refractive index (RI) detection. Chromatographic separation is achieved using 0.01 M H₂SO₄ as the mobile phase with a flow rate of 0.5 mL/min and a column temperature of 50° C. Isobutanol retention time is 47.6 minutes.

The meaning of abbreviations is as follows: “s” means second(s), “min” means minute(s), “h” means hour(s), “psi” means pounds per square inch, “nm” means nanometers, “d” means day(s), “μL” means microliter(s), “mL” means milliliter(s), “L” means liter(s), “mm” means millimeter(s), “nm” means nanometers, “mM” means millimolar, “M” means molar, “mmol” means millimole(s), “μmol” means micromole(s)“, “g” means gram(s), “μg” means microgram(s) and “ng” means nanogram(s), “PCR” means polymerase chain reaction, “OD” means optical density, “OD₆₀₀” means the optical density measured at a wavelength of 600 nm, “kDa” means kilodaltons, “g” means the gravitation constant, “bp” means base pair(s), “kbp” means kilobase pair(s), “% w/v” means weight/volume percent, % v/v″ means volume/volume percent, “wt %” means percent by weight, “HPLC” means high performance liquid chromatography, and “GC” means gas chromatography. The term “molar selectivity” is the number of moles of product produced per mole of sugar substrate consumed and is reported as a percent.

### Example 1

### Expression of DHAD from K. Lactis in LEU1 Deletion Strain of S. Cerevisiae

The yeast LEU1 gene encodes isopropylmalate dehydratase, an enzyme that requires an Fe—S cluster for its function. The impact of LEU1 deletion on DHAD activity expressed from the Kluyveromyces lactis DHAD coding region was examined in this example. For gene expression in yeast, the shuttle vector pNY13 (SEQ ID NO:29) derived from pRS423 was used. This shuttle vector contained an F1 origin of replication (1423 to 1879) for maintenance in E. coli and a 2 micron origin (nt 7537 to 8881) for replication in yeast. The vector has an FBA promoter (nt 2111 to 3110) and FBA terminator (nt 4316 to 5315). In addition, it carries the HIS3 marker (nt 504 to 1163) for selection in yeast and ampicillin resistance marker (nt 6547 to 7404) for selection in E. coli. pNY9 is the same vector with a URA3 marker replacing the HIS3 marker.

The ILV3 coding region for DHAD from Kluyveromyces lactis was synthesized with codon-optimization for expression in S. cerevisiae by DNA 2.0 (Menlo Park, Calif.). The cloned synthesized sequence was PCR amplified. During amplification, a portion of the mitochondrial signal peptide for the DHAD at the N-terminus was deleted by using ilv3(K)(0)-F(delet) as the forward primer with ilv3(K)(o)-R as the reverse primer, resulting in a coding region for cytoplasmic expression (SEQ ID NO:173). In addition, an SphI site was incorporated in the forward primer, while a NotI site was included in the reverse primer. The PCR product was cloned into the shuttle vectors pNY9 and pNY13 so that the ILV3 coding region was under the control of the FBA promoter. Both PCR product and each vector (pNY9, pNY13) were digested with SphI and NotI. After digestion, the components were ligated, and the ligation mixture was transformed into TOP10 competent cells (Invitrogen). Transformants were selected in LB agar plates supplemented with 100 μg/ml of ampicillin. Positive clones were screened by PCR with the forward and reverse primers described above. The resulting plasmids were designated as pRS423::FBAp-ILV3(KL) and pRS426::FBAp-ILV3(KL), derived from plasmids pNY13 and pNY9, respectively.

To study the expression of the DHAD from K. lactis in S. cerevisiae the expression vector pRS423::FBAp-ILV3(KL) along with an empty vector pRS426 were transformed into strains BY4743 and BY4743 leu1::kanMX4 (ATCC 4034377). The competent cell preparation and transformation were based on the Frozen Yeast Transformation kit from Zymo Research. The transformants were selected on agar plates with yeast synthetic medium lacking histidine and uracil (Teknova). For enzymatic assays, the strains carrying the expression construct and the empty vector pRS426 were first grown overnight in 5 ml synthetic complete yeast medium lacking histidine and uracil. The 5 ml overnight cultures were transferred into 100 ml of medium in a 250 ml flask. The cultures were harvested when they reached 1 to 2 O.D. at 600 nm. The samples were washed with 10 ml of 20 mM Tris (pH 7.5) and then resuspended in 1 ml of the same Tris buffer. The samples were transferred into 2.0 ml tubes containing 0.1 mm silica (Lysing Matrix B, MP biomedicals). The cells were then broken in a bead-beater (BIO101). The supernatant was obtained by centrifugation in a microfuge at 13,000 rpm at 4° C. for 30 minutes. Typically, 0.06 to 0.1 mg of crude extract protein was used in a DHAD assay. Protein in the crude extracts was determined by Bradford assay with Coomassie stain.

### Dihydroxy-Acid Dehydratase Enzyme Assay

The in vitro DHAD enzyme assay is a variation on the assay described in Flint et al. (J. Biol. Chem. (1993) 268:14732-14742.). The assay was performed in a 1.6 ml total volume and consisted of: 800 μl 2× buffer (100 mM Tris pH 8.0, 20 mM MgCl₂), 160 μl 10× substrate (15.6 mg/ml dihydroxyisovalerate), crude extract (typically 50-200 μg protein), and water. The reaction was incubated at 37° C. At 0, 30, 60, and 90 minute time intervals, 350 μl aliquots of the reaction were removed and incubated with 350 μl of 0.05% dinitrophenyhydrazine in 1N HCl for 30 minutes at 25° C. To quench the reaction, 350 μl of 4N sodium hydroxide was added to the reaction mixture, and the reaction was centrifuged at 15,000×g for 2 minutes. The supernatant was transferred to a plastic disposable cuvette and absorbance at 540 nm was measured in a spectrophotometer. The amount of α-ketoisovalerate (KIV) produced was determined by entering the absorbance into the linear regression equation obtained from a standard curve of α-ketoisovalerate. The amount of KIV produced at each time point was plotted to determine the rate of production. The slope of the linear regression was then used to calculate specific activity using the formula:

Specific activity calculation=(slope of KIV production/1000)/mg protein per 1.6 mL reaction=mmol/min*mg

The dehydratase from K. lactis had a specific activity in the range of 0.2 to 0.35 μmol min⁻¹ mg⁻¹ when expressed in yeast strain BY4743 (Δleu1). In contrast, this enzyme had a specific activity in the range of only 0.14 μmol min⁻¹ mg⁻¹ when expressed in the parent yeast strain BY4743. Strains BY4743 (Δleu1) and wildtype BY4743 containing empty vectors pRS423 or pRS426 had a background of activity in the range of 0.03 to 0.1 μmol min⁻¹ mg⁻¹.

### Example 2

### Expression of Diol Dehydratase in LEU1 Deletion Strain of S. Cerevisiae

A coenzyme B12-independent propanediol dehydratase is disclosed in commonly owned and co-pending US Patent Pub No. US20090155870. The sequences encoding this coenzyme B12-independent (S-adenosylmethionine (SAM)-dependent) propanediol dehydratase (SEQ ID NO:15) and its putative associated reactivase (SEQ ID NO:16) in the bacterium Roseburia inulinivorans [Scott et al.(2006) J. Bacteriol.188:4340-9], hereafter referred to as rdhtA and rdhtB, respectively, were synthesized as one DNA fragment (SEQ ID NO:17) by standard methods and cloned into an E. coli vector (by DNA2.0, Inc., Menlo Park, Calif.). This clone was used as a PCR template to prepare separate RdhtA and RdhtB coding region fragments. The RdhtA coding region for the diol dehydratase was amplified by PCR using primers N695 and N696 (SEQ ID NOs:18 and 19). The RdhtB coding region for the diol dehydratase activase, was amplified by PCR using primers N697 and N698 (SEQ ID NOs:20 and 21). The two DNA fragments were combined with a dual terminator DNA fragment (SEQ ID NO:22) that has an ADH terminator (SEQ ID NO:23) and a CYC1 terminator (SEQ ID NO:24) adjacent to each other in opposing orientation using SOE PCR (Horton et al. (1989) Gene 77:61-68). The dual terminator fragment was isolated as a 0.6 kb fragment following Pacl digestion of pRS426::FBA-ILV5+GPM-kivD (described in co-owned and co-pending US Patent Publication 20070092957 A1, Example 17). The resulting 4 kb DNA fragment had the RdhtA and RdhtB coding regions in opposing orientation on either side of the dual terminator, with the 3′end of each coding region adjacent to the dual terminator sequence. This DNA fragment was then cloned by gap repair methodology (Ma et al. (1987) Genetics 58:201-216) into the S. cerevisiae shuttle vector pRS426::FBA-ILV5+GPM-kivD that was prepared by digestion with BbvCl to remove the ILV5 and kivD coding regions and dual terminator sequence between their 3′ ends. The resulting plasmid, pRS426::RdhtAB (below), contained the RdhtA gene under the control of the S. cerevisiae FBA promoter (SEQ ID NO:25) and the RdhtB gene under control of the S. cerevisiae GPM promoter (SEQ ID NO:26).

Plasmids pRS426 and pRS426::RdhtAB were introduced into S. cerevisiae strains BY4743 (ATCC 201390) and BY4743 leu1::kanMX4 (ATCC 4034377) by standard techniques (Methods in Yeast Genetics, 2005, Cold Spring Harbor Laboratory Press, Cold Spring Harbor, N.Y., pp. 201-202). Cells were plated on synthetic complete medium lacking uracil to select for transformants. Transformants were tested for diol dehydratase activity using an in vivo assay as follows. Patched cells grown on solid medium were used to inoculate liquid media (20 ml) in petri plates. Media used were synthetic complete minus uracil with and without addition of 5 g/L 1,2-propanediol 9Aldrich Cat. No. 398039). The petri plates were transferred to an Anaeropack™ System jar (Mitsubishi Gas Chemical Co. Cat. No.50-70). An anaerobic environment (<0.1% oxygen) was generated using Pack-Anaero sachets (Mitsubishi Gas Chemical Co. Cat. No. 10-01). After 48 hours, culture supernatants were sampled, filtered and analyzed by HPLC as described in General Methods. Propanol, which has a retention time of 38.8 minutes, was observed in culture supernatants of strains carrying pRS426::RdhtAB when 1,2-propanediol was provided in the medium. The results given in Table 5 show that more propanol was produced in the supernatants of the strain also carrying the LEU1 deletion than in the strain without the LEU1 deletion. Statistical analysis gave a P score of less than 0.0005.

TABLE 5
Propanol production with propanediol dehydratase/reactivase
expression in yeast with and without LEU1 knockout.

| | 1,2-propanediol | Propanol |
|---|---|---|
| Strain | Added | Peak Area |
| BY4743 | 5 g/L | 19472 ± 1403 |
| Δleu1::kanMX4/pRS426::RdhtAB | | (n = 6) |
| BY4743 | 0 g/L | 2478 |
| Δleu1::kanMX4/pRS426::RdhtAB | | (n = 1) |
| BY4743/pRS426::RdhtAB | 5 g/L | 11830 ± 1983 |
| | | (n = 6) |
| BY4743/pRS426::RdhtAB | 0 g/L | 2369 |
| | | (n = 1) |
| BY4743 Δleu1::kanMX4/pRS426 | 5 g/L | 2633 |
| | | (n = 1) |
| BY4743/pRS426 | 5 g/L | 2841 |
| | | (n = 1) |

### Example 3

### Improving Cytosolic Dihydroxy-Acid Dehydratase (DHAD) Activity in S. Cerevisiae Through a Disruption of Mitochondrial ILV3

### Vector/Host Construction

In S. cerevisiae ILV3 encodes the mitochondrial dihydroxy-acid dehydratase that is involved in branched chain amino acid biosynthesis. To reduce background from endogenous ILV3 expression for in vitro enzymatic assays in S. cerevisiae, an ilv3::URA3 disruption cassette was constructed by PCR amplification of the URA3 marker from pRS426 (ATCC No. 77107) with primers “ILV3::URA3 F” and “ILV3::URA3 R”, given as SEQ ID NO:30 and 31. These primers produced a 1.4 kb URA3 PCR product that contained 70 by 5′ and 3′ extensions identical to sequences upstream and downstream of the ILV3 chromosomal locus for homologous recombination. The PCR product was transformed into BY4741 cells (ATCC 201388) using standard genetic techniques (Methods in Yeast Genetics, 2005, Cold Spring Harbor Laboratory Press, Cold Spring Harbor, N.Y., pp. 201-202) and resulting transformants were maintained on synthetic complete media lacking uracil and supplemented with 2% glucose at 30° C. Transformants were screened by PCR using primers “ILV3 F Check” and “URA3 REV Check”, given as SEQ ID NOs:32 and 33, to verify integration at the correct site and disruption of the endogenous ILV3 locus. The correct transformants had the genotype: BY4741 ilv3::URA3.

Construction of plasmid pRS423::FBAp-ILV3(KL) and pRS426::FBAp-ILV3(KL) were described in Example 1. Construction of pRS423::CUP1-alsS+FBA-ILV3 has been described in co-owned and co-pending US Patent Publication US20070092957 A1, Example 17 which is herein incorporated by reference. pRS423::CUP1-alsS+FBA-ILV3 is the same plasmid as pRS423::CUP1p-alsS-FBAp-ILV3. This construction contains a chimeric gene containing the S. cerevisiae CUP1 promoter (SEQ ID NO:34), alsS coding region from Bacillus subtilis (SEQ ID NO:27), and CYC1 terminator (SEQ ID NO:24); and also a chimeric gene containing the S. cerevisiae FBA promoter (SEQ ID NO:25), ILV3 coding region from S. cerevisiae lacking the mitochondrial targeting signal coding sequence (SEQ ID NO:111) and ADH1 terminator (SEQ ID NO:23).

### Preparation of Samples

Plasmid vectors pRS423::CUP1p-alsS-FBAp-ILV3 and pRS423::FBAp-ILV3(KL) were transformed into strain BY4741 ilv3::URA3 using standard genetic techniques (Methods in Yeast Genetics, 2005, Cold Spring Harbor Laboratory Press, Cold Spring Harbor, N.Y.) and maintained on synthetic complete media lacking histidine. Plasmid vectors pRS423::CUP1p-alsS-FBAp-ILV3 and pRS426::FBAp-ILV3(KL) were also transformed into strain BY4741. Aerobic cultures were grown in 1000 ml flasks containing 200 ml synthetic complete media lacking histidine and supplemented with 2% glucose in an Innova4000 incubator (New Brunswick Scientific, Edison, N.J.) at 30° C. and 225 rpm. Cultures were harvested at OD600 measurements of 1.0-2.0 and pelleted by centrifugation at 6000×g for 10 minutes. Cell pellets were washed with 10 mM Tris-HCl, pH 8.0 and pellets were stored at −80° C. until assayed for activity. Cell free extracts were prepared by standard bead beating method using 1 ml of 0.5 mm beads and 1.5 ml of yeast cell suspension. Protein concentration in the extracts was determined by Bradford assay with Coomassie stain. DHAD enzyme assays and specific activity calculations were performed as described in Example 1. The results given in Table 6 show that there was higher DHAD activity in the ILV3 deletion cells than in cells without the ILV3 deletion.

TABLE 6
DHAD activity in yeast with and without ILV3 deletion.

| | | Average |
|---|---|---|
| | Specific | Specific |
| | Activity | Activity |
| Strain | (μmol/min*mg) | (μmol/min*mg) |
| BY4741 | 0.018 | 0.013 |
| | 0.014 | |
| | 0.008 | |
| BY4741 pRS423::CUP1p-alsS-FBAp- | 0.018 | 0.019 |
| ILV3 | | |
| | 0.020 | |
| BY4741 pRS426::FBAp-ILV3(KL) | 0.040 | 0.038 |
| | 0.036 | |
| BY4741 ilv3::URA3 | 0.00006 | 0.00041 |
| | 0.00075 | |
| BY4741 ilv3::URA3 pRS423::CUP1p- | 0.030 | 0.029 |
| alsS-FBAp-ILV3 | | |
| | 0.028 | |
| BY4741 ilv3::URA3 pRS423::FBAp- | 0.317 | 0.281 |
| ILV3(KL) | | |
| | 0.244 | |

### Verification of Alpha-Ketoisovalerate Formation by HPLC

Formation of alpha-ketoisovalerate from the in vitro DHAD enzyme assays was accomplished using HPLC and semicarbizide derivatization. DHAD enzyme assays were performed in a 1.6 ml total volume and consisted of: 800 μl 2× buffer (100mM Tris pH 8.0, 20mM MgCl₂), 160 μl 10×substrate (15.6 mg/m I dihydroxyisovalerate), crude extract (typically 50-200 μg protein), and water. The reactions were incubated at 37° C. At time intervals of zero and 90 minutes 350 μl aliquots of the reactions were removed, transferred to ice, and centrifuged at 13,000×g for 2 minutes at 4° C. to remove precipitated protein. The supernatants were transferred to ice-chilled Microcon YM-10 (Sigma) spin columns and centrifuged at 13,000 ×g for 20 minutes at 4° C. to remove enzymes and soluble proteins. The flowthroughs were mixed with 100 μl derivatizing reagent (1% semicarbizide hydrochloride and 1.5% sodium acetate trihydrate) and incubated at room temperature for 15 minutes. The reactions were spun through CoStar spin filters (CoStar, 0.22 μm filter) at 13,000×g for 5 minutes at 4° C. to remove any precipitates. The flowthroughs were transferred to HPLC vials for analysis.

Analysis of derivatized alpha-ketoisovalerate was conduced using reverse phase chromatography on a Supelco LC-18 column with Superguard LC-18-DB guard column (Supelco; 25 cm×4.6 mm, 5 μm). Injection volumes were 10 μl. Mobile phases were methanol (A) and 50 mM NaOAc pH 7.2. The gradient program utilized is given in Table 7, with detection at 250 nm.

TABLE 7
Gradient used for derivatized alpha-ketoisovalerate HPLC assay

| Time | Flow | % NaOAc | | |
|---|---|---|---|---|
| (min) | (ml/min) | (50 mM) | % MEOH | Curve |
| Initial | 1.0 | 95 | 5 | |
| 5 | 1.0 | 95 | 5 | 6 |
| 20 | 1.0 | 70 | 30 | 6 |
| 21 | 1.0 | 0 | 100 | 6 |
| 25 | 1.0 | 0 | 100 | 6 |
| 26 | 1.0 | 95 | 5 | 6 |
| 35 | 1.0 | 95 | 5 | 6 |

The retention time of semicarbizide-derivatized alpha-ketoisovalerate was 11.5 minutes.

The results, which are given in Table 8, confirmed that KIV was produced in the cells, as detected in the indirect assay for specific activity above. The amount of KIV listed in the DHAD Assay column is the amount determined indirectly in the 90 min sample for the activity assay described above in determining the specific activity. This amount of KIV correlates well with the amount detected in the HPLC assay.

TABLE 8
Comparison of KIV detected in DHAD
activity assay and by HPLC.

| | keto-isovalerate production (μM) | |
|---|---|---|
| Strain | DHAD Assay | HPLC |
| BY4741 ilv3::URA3 pRS423::FBAp- | 256 | 256 |
| ILV3(KL) | | |
| BY4741 ilv3::URA3 pRS423::FBAp- | 162 | 163 |
| ILV3(KL) | | |

### Example 4

### Expression of Dihydroxy-Acid Dehydratase from S. Mutans in Yeast

The shuttle vector pRS423 FBA ilvD(Strep) (SEQ ID NO:194) was used for the expression of DHAD from Streptococcus mutans. This shuttle vector contained an F1 origin of replication (1423 to 1879) for maintenance in E. coli and a 2 micron origin (nt 8082 to 9426) for replication in yeast. The vector has an FBA promoter (nt 2111 to 3108; SEQ ID NO:195) and FBA terminator (nt 4861 to 5860). In addition, it carries the His marker (nt 504 to 1163) for selection in yeast and ampicillin resistance marker (nt 7092 to 7949) for selection in E. coli. The ilvD coding region (nt 3116 to 4828) from Streptococcus mutans UA159 (ATCC 700610) is between the FBA promoter and FBA terminator forming a chimeric gene for expression.

To test the expression of the DHAD from Streptococcus mutans in yeast strain BY4741 (known in the art and obtainable from ATCC #201388), the expression vector pRS423 FBA IlvD(Strep) was transformed in combination with empty vector pRS426 into BY4741 cells (obtainable from ATCC, # 201388). The transformants were grown on synthetic medium lacking histidine and uracil (Teknova). Growth on liquid medium for assay was carried out by adding 5 ml of an overnight culture into 100 ml medium in a 250 ml flask. The cultures were harvested when they reached 1 to 2 O.D. at 600 nm. The samples were washed with 10 ml of 20 mM Tris (pH 7.5) and then resuspended in 1 ml of the same Tris buffer. The samples were transferred into 2.0 ml tubes containing 0.1 mm silica (Lysing Matrix B, MP biomedicals). The cells were then broken in a bead-beater (BIO101). The supernatant was obtained by centrifugation in a microfuge at 13,000 rpm at 4° C. for 30 minutes. Typically, 0.06 to 0.1 mg of crude extract protein was used in DHAD assay at 37° C. as described by Flint and Emptage (J. Biol. Chem. (1988) 263(8): 3558-64) using dinitrophenylhydrazine. The dehydratase from Streptococcus mutans had a specific activity of 0.24 μmol min⁻¹ mg⁻¹ when expressed in yeast. A control strain containing empty vectors pRS423 and pRS426 had a background of activity in the range of 0.03 to 0.06 μmol min⁻¹ mg⁻¹.

### Example 5

### Expression of the IlvD Gene from L. Lactis in Yeast

The ilvD coding region from L. lactis was amplified with the forward primer IlvD(LI)-F (SEQ ID NO:196) and reverse primer IlvD(LI)-R (SEQ ID NO:197). The amplified fragment was cloned into the shuttle vector pNY13by gap repair. pNY13 (SEQ ID NO:198) was derived from pRS423. This shuttle vector contained an F1 origin of replication (1423 to 1879) for maintanence in E. coli and a 2 micron origin (nt 7537 to 8881) for replication in yeast. The vector has an FBA promoter (nt 2111 to 3110) and FBA terminator (nt 4316 to 5315). In addition, it carries the His marker (nt 504 to 1163) for selection in yeast and Ampicillin resistance marker (nt 6547 to 7404) for selection in E. coli.

Positive clones were selected based on amplification with the forward and reverse primers for the ilvD coding region and further confirmed by sequencing. The new construct was designated as pRS423 FBA ilvD(L. lactis). This construct was transformed into yeast strain BY4743 (ΔLEU1) (Open Biosystems, Huntsville, Ala.; Catalog #YSC1021-666629) along with the empty vector pRS426 as described in Example 6. The growth and assay of yeast strains containing the expression vector were also carried out according to the procedures as described in Example 4. The dihydroxy-acid dehydratase activity in yeast strains with the L. lactis IlvD gene was determined to be in the range of 0.05 to 0.17 μmol min⁻¹ mg⁻¹. This activity was slightly above the control. Complementation experiments were carried out to investigate the expression of this DHAD and DHADs from other bacteria (example 8).

### Example 6

### Complementation of Yeast ILV3 Deletion Strain with Bacterial DHADs.

The endogenous DHAD enzyme of S. cerevisiae is encoded by the ILV3 gene and the protein is targeted to the mitochondrion. Deletion of this gene results in loss of endogenous DHAD activity and provides a test strain where expression of heterologous cytosolic DHAD activity can be readily assessed. Deletion of ILV3 results in an inability of the strain to grow in the absence of branched-chained amino acids. Expression of different bacterial DHADs was assayed by determining their ability to complement the yeast ILV3 deletion strain such that it grows in the absence of branched-chained amino acids.

Expression shuttle vectors containing ilvD gene sequences encoding DHADs from the bacteria listed in Table 10 were constructed. The basic elements of these expression constructs were the same as the pRS423 FBA ilvD(strep) vector described in Example 4. Each of the ilvD coding regions was prepared by PCR as described in Example 2 of US Patent Application publication US20100081154A1, which is herein incorporated by reference, and cloned to replace the Streptococcus mutans ilvD coding region in pRS423 FBA ilvD(Strep) creating the plasmids listed in Table 10. These expression constructs were transformed into the ILV3 deletion strain, BY4741 ilv3::URA3 and was prepared as follows. An ilv3::URA3 disruption cassette was constructed by PCR amplification of the URA3 marker from pRS426 (ATCC No. 77107) with primers “ILV3::URA3 F” and “ILV3::URA3 R”, given as SEQ ID NOs: 199 and 200. These primers produced a 1.4 kb URA3 PCR product that contained 70 by 5′ and 3′ extensions identical to sequences upstream and downstream of the ILV3 chromosomal locus for homologous recombination. The PCR product was transformed into BY4741 cells (ATCC 201388) using standard genetic techniques (Methods in Yeast Genetics, 2005, Cold Spring Harbor Laboratory Press, Cold Spring Harbor, N.Y., pp. 201-202) and resulting transformants were maintained on synthetic complete media lacking uracil and supplemented with 2% glucose at 30° C. Transformants were screened by PCR using primers “ILV3 F Check” and “URA3 REV Check”, given as SEQ ID NOs:201 and 202, to verify integration at the correct site and disruption of the endogenous ILV3 locus.

The transformants with bacterial DHADs in Table 10 were selected on plates with yeast synthetic medium lacking histidine. Colonies selected were then patched onto plates lacking valine, leucine and isoleucine. Strains containing the expression vectors listed in Table 10 were able to grow on these plates lacking the branched chain amino acids, while the control strain with the control plasmid did not. This result indicated that DHADs from these bacteria were actively expressed in S. cerevisiae.

TABLE 10
Bacterial DHADs tested and Expression vectors

| | SEQ ID NO. | |
|---|---|---|
| | OF DHAD | |
| Organism Source | nucleic acid | Vector |
| of DHAD | sequence | designation |
| Nitrosomonas europaea ATCC | 174 | pRS423 FBA ilvD |
| 19718 | | (europ) |
| Synechocystis sp PCC 6803 | 176 | pRS423 FBA ilvD |
| | | (Synech) |
| Streptococcus thermophilus | 180 | pRS423 FBA ilvD |
| LMG 18311 | | (thermo) |
| Ralstonia eutropha H16 | | pRS423 FBA ilvD |
| (ATCC 17699) | | (H16) |
| Lactococcus lactis | 186 | pRS423 FBA ilvD |
| | | (L. lactis) |

TABLE 9

| | |
|---|---|
| HMMER2.0 [2.2 g] | Program name and version |
| NAME dhad_for_hmm | Name of the input sequence alignment file |
| LENG 564 | Length of the alignment: include indels |
| ALPH Amino | Type of residues |
| MAP yes | Map of the match states to the columns of the alignment |
| COM/app/public/hmmer/current/bin/hmmbuild -F dhad-exp_hmm dhad_for_hmm.aln | Commands used to generate the file: this one means that hmmbuild (default patrameters) was applied to |
| | the alignment file |
| COM/app/public/hmmer/current/bin/hmmcalibrate dhad-exp_hmm | Commands used to generate the file: this one means that hmmcalibrate (default parametrs) was applied to |
| | the hmm profile |
| NSEQ 8 | Number of sequences in the alignment file |
| DATE Tue Jun. 3 10:48:24 2008 | When was the file generated |
| XT −8455 −4 −1000 −1000 −8455 −4 −8455 −4 | |
| NULT −4 −8455 | The transition probability distribution for the null model (single G state). |
| | The symbol emission probability distribution for the null model (G state); consists of K (e.g. 4 or 20) |
| NULE 595 −1558 85 338 −294 453 −1158 197 249 902 −1085 −142 −21 −313 45 | integers. The null probability used to convert these back to model probabilities is 1/K. |
| 531 201 384 −199 | |
| | The extreme value distribution parameters μ and lambda respectively; both floating point values. |
| EVD −499.650970 0.086142 | Lambda is positive and nonzero. These values are set when the model is calibrated with hmmcalibrate. |

| | | | | | | | | | | | | | | | | | | | | | Position in |
|---|---|---|---|---|---|---|---|---|---|---|---|---|---|---|---|---|---|---|---|---|---|
| HMM | A | C | D | E | F | G | H | I | K | L | M | N | P | Q | R | S | T | V | W | Y | alignment |
| | m−>m | m−>i | m−>d | i−>m | i−>i | d−>m | d−>d | b−>m | m−>e | | | | | | | | | | | | |
| | −538 | * | −1684 | | | | | | | | | | | | | | | | | | |
| 1(M) | −233 | −1296 | 99 | 1223 | −1477 | −1132 | 89 | −1122 | 420 | −1248 | 1757 | 1553 | −1296 | 464 | −24 | −190 | −188 | −838 | −1578 | −985 | 6 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −29 | −6203 | −7245 | −894 | −1115 | −701 | −1378 | −538 | * | | | | | | | | | | | | |
| 2(E) | −220 | −1288 | 232 | 1356 | −1807 | 1016 | −70 | −1474 | 190 | −1584 | −775 | 132 | −1298 | 300 | −282 | −183 | 1140 | −1092 | −1872 | −1262 | 7 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −29 | −6203 | −7245 | −894 | −1115 | −701 | −1378 | * | * | | | | | | | | | | | | |
| 3(K) | −448 | −1932 | 1558 | 658 | −2220 | −1048 | 40 | −1983 | 1569 | −1938 | −1091 | 1558 | −1319 | 450 | −193 | −278 | −419 | −1552 | −2121 | −1397 | 8 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −29 | −6203 | −7245 | −894 | −1115 | −701 | −1378 | * | * | | | | | | | | | | | | |
| 4(V) | −404 | −498 | −1497 | −939 | −588 | −1810 | −640 | 1591 | 914 | −127 | 335 | −962 | −1866 | −562 | −767 | −868 | −357 | 1720 | −1169 | −763 | 9 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −29 | −6203 | −7245 | −894 | −1115 | −701 | −1378 | * | * | | | | | | | | | | | | |
| 5(E) | −265 | −1340 | −52 | 1376 | −1572 | −1189 | 113 | −1125 | 1345 | −1287 | −496 | 99 | −1321 | 505 | 198 | −218 | −205 | 597 | −1598 | −1032 | 10 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −29 | −6203 | −7245 | −894 | −1115 | −701 | −1378 | * | * | | | | | | | | | | | | |
| 6(S) | 256 | −397 | −1014 | −830 | −1841 | −646 | −862 | −1443 | −767 | −1740 | −963 | −568 | −1249 | −651 | −1007 | 2267 | 1586 | −862 | −2080 | −1672 | 11 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −29 | −6203 | −7245 | −894 | −1115 | −701 | −1378 | * | * | | | | | | | | | | | | |
| 7(M) | −990 | −889 | −2630 | 157 | −513 | −2514 | −1346 | 1309 | −1767 | 820 | 3683 | −1898 | −2491 | −1496 | −1799 | −1589 | −925 | 150 | −1336 | −1041 | 12 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 8(E) | 588 | −1875 | −194 | 1536 | −2188 | −1373 | −59 | −1931 | 957 | −1890 | −977 | 904 | 292 | 393 | −162 | 483 | −372 | −1495 | −2070 | −1391 | 13 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 9(N) | −514 | −1116 | 1207 | −315 | 447 | −1650 | −304 | −778 | −224 | 825 | −277 | 1457 | −1738 | −123 | −618 | −627 | −454 | −603 | −1186 | 763 | 14 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 10(N) | −815 | −1190 | −1360 | −922 | −904 | −1967 | −797 | −442 | −670 | 381 | 1700 | 3009 | −2099 | −654 | −934 | −1051 | −791 | −445 | −1490 | −979 | 15 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 11(K) | −1530 | −2498 | −1722 | −855 | −3141 | −2246 | −428 | −2627 | 2828 | −2404 | −1656 | −927 | 662 | −2 | 2047 | −1421 | −1337 | −2324 | −2357 | −2081 | 16 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 12(Y) | −872 | −1887 | −861 | −290 | −1369 | −1801 | 1662 | −1797 | 325 | −1793 | −1031 | 893 | −1876 | 56 | 2219 | −812 | −780 | −1514 | −1565 | 2287 | 17 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 13(S) | −830 | −1586 | −1471 | −1099 | −2717 | −1642 | −1010 | −2479 | −266 | −2518 | −1746 | −1065 | −2069 | −676 | 1822 | 2748 | −1000 | −1950 | −2597 | −2189 | 18 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 14(Q) | −851 | −2131 | −775 | −153 | −2554 | −1735 | −211 | −2205 | 1908 | −2094 | −1244 | −386 | −1802 | 2254 | 974 | 1001 | −747 | −1819 | −2181 | −1667 | 19 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 15(T) | −405 | −1258 | −618 | −100 | −1490 | −1466 | 1158 | −1121 | 1 | −1299 | −514 | 578 | −1607 | 65 | −433 | 960 | 1849 | 343 | −1677 | −1143 | 20 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 16(I) | −1772 | −1325 | −4307 | −3877 | −1405 | −3993 | −3383 | 2935 | −3705 | 820 | −217 | −3632 | −3761 | −3400 | −3682 | −3260 | −1742 | 2033 | −2838 | −2525 | 21 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 17(T) | −1018 | −1329 | −2004 | −1771 | −409 | −1993 | −1000 | −1256 | −1512 | −1464 | −966 | −1543 | −2367 | −1428 | −1638 | −1257 | 3050 | −1090 | −1012 | 2448 | 22 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 18(Q) | −1509 | −3056 | 1970 | 44 | −3310 | −1666 | −896 | −3242 | −877 | −3158 | −2439 | −322 | −2123 | 3562 | −1493 | −1259 | −1550 | −2779 | −3260 | −2446 | 23 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 19(D) | −1006 | −2199 | 2178 | −88 | −3159 | 1997 | −936 | −2974 | −948 | −2977 | −2174 | −382 | −1960 | −589 | −1571 | 1295 | −1157 | −2369 | −3178 | −2430 | 24 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 20(M) | 445 | −796 | −1082 | −521 | −841 | −1643 | −412 | −403 | −370 | −692 | 2213 | −646 | 536 | 1166 | −698 | −630 | 660 | 831 | −1204 | −767 | 25 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 21(Q) | 741 | −990 | −1025 | −507 | −1249 | −1551 | −519 | −720 | −357 | −1062 | −345 | −635 | −1739 | 1770 | −713 | −589 | 1576 | 1129 | −1559 | −1097 | 26 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 22(R) | −1753 | −2648 | −2072 | −1047 | −3365 | −2405 | −452 | −2782 | 1989 | −2495 | −1773 | −1062 | −2379 | 2402 | 2643 | −1629 | −1506 | −2504 | −2397 | −2190 | 27 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 23(S) | −330 | −1010 | −1820 | −1628 | −2778 | −1229 | −1652 | −2481 | −1592 | −2691 | −1841 | −1273 | 2130 | −1426 | −1834 | −2449 | 1034 | −1716 | −2961 | −2594 | 28 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 24(P) | 1882 | −1119 | −2231 | −2302 | −3062 | −1360 | −2209 | −2710 | −2339 | −3013 | −2243 | −1676 | 3304 | −2117 | −2409 | −742 | −918 | −1916 | −3263 | −3022 | 29 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 25(N) | 969 | −1230 | −1066 | −915 | −2593 | −1313 | −1196 | −2242 | −1033 | −2447 | −1626 | 3197 | −1850 | −898 | −1392 | −582 | 1155 | −1644 | −2736 | −2256 | 30 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 26(R) | −1847 | −2640 | −2014 | −1161 | −3282 | −2428 | −579 | −2818 | 687 | −2553 | −1869 | −1165 | −2462 | 2447 | 3181 | −1746 | −1630 | −2555 | −2447 | −2228 | 31 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 27(A) | 3048 | −932 | −2480 | −2533 | −3075 | −1200 | −2274 | −2765 | −2501 | −3071 | −2221 | −1658 | −1948 | −2205 | −2512 | 1225 | −739 | −1842 | −3322 | −3078 | 32 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 28(M) | −2406 | −2296 | −3638 | −3594 | −1525 | −3105 | −2824 | −1047 | −3121 | −596 | 5043 | −3293 | −3425 | −3046 | −2996 | −2911 | −2552 | −1398 | −2513 | −2207 | 33 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 29(Y) | −1674 | −1506 | −2863 | −2464 | 596 | −2872 | 2251 | −972 | −2024 | 2197 | −552 | −1986 | −2876 | −1739 | −1988 | −1987 | −1601 | −1002 | −95 | 2332 | 34 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 30(Y) | −2013 | −2305 | −2428 | −1781 | −328 | −2709 | −654 | −2240 | −258 | −2064 | −1626 | −1631 | −2788 | −899 | 2789 | −2017 | −1896 | −2130 | −857 | 3434 | 35 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 31(A) | 2822 | −1031 | −2418 | −2539 | −3226 | 1898 | −2364 | −2941 | −2626 | −3229 | −2379 | −1722 | −2026 | −2302 | −2634 | −654 | −848 | −1983 | −3415 | −3226 | 36 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 32(I) | −1247 | −941 | −3569 | −3039 | −1082 | −3101 | −2185 | 2227 | −2763 | 766 | −76 | −2700 | −3050 | −2469 | −2697 | −2253 | 1322 | 1974 | −1988 | −1633 | 37 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 33(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 38 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 34(F) | −1511 | −1236 | −3511 | −3017 | 2747 | −2982 | −1069 | −260 | −2651 | 992 | 2737 | −2407 | −2904 | −2088 | −2418 | −2099 | −1434 | −489 | −537 | 2056 | 39 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 35(Q) | −576 | −1869 | −401 | 92 | −2232 | 831 | −173 | −1930 | 1505 | −1913 | −1042 | −186 | −1620 | 1653 | −51 | −482 | 1346 | −1534 | −2098 | −1490 | 40 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 36(D) | −1352 | −3066 | 3028 | 1349 | −3303 | −1566 | −724 | −3141 | 1155 | −3043 | −2267 | −165 | −1991 | −354 | −1350 | −1086 | −1368 | −2659 | −3221 | −2356 | 41 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 37(E) | −1507 | −3288 | 2042 | 2762 | −3520 | 515 | −853 | −3401 | −981 | −3296 | −2566 | −182 | −2064 | −503 | −1753 | −1209 | −1553 | −2895 | −3486 | −2547 | 42 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 38(D) | −1445 | −2778 | 3529 | −53 | −3524 | −1590 | −1129 | −3476 | −1367 | −3459 | −2774 | −396 | −2156 | −825 | −2122 | 554 | −1609 | −2880 | −3582 | −2717 | 43 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 39(F) | −2658 | −2176 | −4213 | −4000 | 3815 | −3933 | −1352 | −531 | −3638 | 1121 | −19 | −3184 | −3709 | −2820 | −3296 | −3219 | −2579 | −1037 | −601 | 403 | 44 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 40(D) | −684 | −2193 | 1738 | 1460 | −2494 | −1437 | −249 | −2257 | 1694 | −2199 | −1308 | −62 | −1637 | 185 | −450 | −531 | 633 | −1808 | −2374 | −1657 | 45 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 41(K) | −2620 | −2961 | −2461 | −2046 | −3743 | −2791 | −1570 | −3603 | 3784 | −3387 | −2839 | −2048 | −3039 | −1260 | −465 | −2604 | −2536 | −3331 | −3001 | −2988 | 46 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 42(P) | 1882 | −1119 | −2231 | −2302 | −3062 | −1360 | −2209 | −2710 | −2339 | −3013 | −2243 | −1676 | 3304 | −2117 | −2409 | −742 | −918 | −1916 | −3263 | −3022 | 47 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 43(I) | −1006 | −992 | −2347 | −1784 | −650 | −2452 | −1256 | 2372 | −1386 | 77 | 2213 | −1720 | −2455 | 2030 | −1490 | −1528 | −946 | 106 | −1441 | −1111 | 48 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 44(V) | −1771 | −1603 | −3750 | −3689 | −2037 | −3050 | −3231 | 403 | −3479 | −1154 | −1076 | −3246 | −3399 | −3383 | −3437 | −2628 | −1917 | 3536 | −3074 | −2677 | 49 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 45(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 50 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 46(I) | −1759 | −1303 | −4330 | −3968 | −1751 | −4051 | −3743 | 3027 | −3837 | −597 | −528 | −3729 | −3875 | −3688 | −3910 | −3369 | −1751 | 2438 | −3259 | −2819 | 51 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 47(V) | 1736 | −1012 | −3546 | −3078 | −1377 | −3073 | −2434 | 2052 | −2843 | −608 | −331 | −2754 | −3122 | −2619 | −2855 | −2270 | −1277 | 2193 | −2333 | −1941 | 52 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 48(N) | −686 | −1511 | −702 | −806 | −2927 | −1386 | −1339 | −2841 | −1264 | −2950 | −2137 | 2702 | −1979 | −1062 | −1648 | 2444 | −971 | −2105 | −3054 | −2475 | 53 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 49(M) | −411 | −857 | −1800 | −1434 | −1528 | 1914 | −1202 | −1029 | −1247 | −1347 | 2989 | −1217 | −1912 | −1119 | −1444 | −676 | 1550 | −767 | −1922 | −1539 | 54 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 50(W) | −782 | −1258 | 793 | −683 | 1193 | 346 | 2051 | −932 | −556 | −1092 | −441 | −798 | −1993 | −426 | −909 | −904 | −720 | −779 | 3163 | 1546 | 55 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 51(W) | 1009 | −798 | −1470 | −935 | −463 | −1773 | −545 | −460 | −751 | −736 | −66 | −943 | −1904 | −606 | −1002 | 1604 | −507 | −322 | 2535 | 1521 | 56 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 52(D) | −1137 | −2711 | 2125 | 1647 | −2995 | −1523 | −617 | −2786 | −528 | −2743 | −1933 | −150 | −1897 | −234 | −1165 | −924 | 2117 | −2331 | −2948 | −2141 | 57 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 53(I) | −599 | −1102 | −1031 | −829 | −1522 | 1429 | −927 | 2119 | −880 | −1369 | −699 | 1692 | −1938 | −759 | −1188 | −799 | −698 | −689 | −1887 | −1419 | 58 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 54(T) | −666 | −1412 | −954 | −984 | −2702 | −1428 | −1357 | −2418 | −1208 | −2650 | −1886 | 2293 | −2000 | −1101 | −1519 | −787 | 2967 | −1835 | −2866 | −2360 | 59 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 55(P) | −632 | −1230 | −2074 | −2144 | −2996 | −1453 | −2116 | −2631 | −2128 | −2928 | −2213 | −1658 | 3610 | −2006 | −2221 | −852 | 1302 | −1931 | −3185 | −2917 | 60 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 56(C) | −2476 | 3735 | −4102 | −4358 | −3712 | −2763 | −3545 | −3518 | −4167 | −3859 | −3569 | −3631 | −3363 | −4030 | −3832 | −2793 | −2860 | −3158 | −3464 | −3718 | 61 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 57(N) | −2171 | −2655 | −1458 | −1748 | −3334 | −2364 | −2267 | −3943 | −2365 | −3936 | −3437 | 4205 | −2932 | −2205 | −2608 | −2224 | −2439 | −3392 | −3253 | −2909 | 62 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 58(M) | 672 | −918 | −3119 | −2578 | −742 | −2668 | −1734 | 1807 | −2263 | 16 | 3713 | −2271 | −2704 | −1960 | −2216 | −1806 | −1058 | 493 | −1612 | −1306 | 63 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 59(H) | −1525 | −2164 | −1235 | −1346 | −2509 | 2296 | 4235 | −3172 | −1516 | −3178 | −2523 | −1448 | −2541 | −1520 | −1760 | −1591 | −1741 | −2656 | −2681 | −2065 | 64 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 60(L) | −2478 | −2009 | −4717 | −4196 | −568 | −4424 | −3262 | 1334 | −3887 | 2824 | 604 | −4085 | −3872 | −3088 | −3590 | −3717 | −2380 | −199 | −2217 | −2207 | 65 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 61(H) | −682 | −2191 | 1015 | 275 | −2485 | 396 | 2379 | −2251 | 62 | −2197 | −1307 | 1826 | −1636 | 1527 | −480 | −529 | −641 | −1803 | −2375 | −1654 | 66 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 62(D) | −575 | −1920 | 1979 | 184 | −2299 | 94 | −242 | −2029 | 114 | −2023 | −1144 | −120 | −1608 | 186 | 1063 | −469 | 1413 | −1605 | −2229 | −1561 | 67 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 63(L) | −2618 | −2139 | −4597 | −4163 | 2144 | −4285 | −2334 | −83 | −3854 | 2690 | 538 | −3771 | −3806 | −2950 | −3488 | −3563 | −2505 | −751 | −1442 | −808 | 68 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 64(A) | 2657 | −1033 | −2408 | −2532 | −3233 | 2193 | −2364 | −2950 | −2626 | −3237 | −2386 | −1719 | −2027 | −2301 | −2635 | −655 | −850 | −1988 | −3420 | −3231 | 69 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 65(K) | −443 | −1857 | 958 | 270 | −2158 | −1393 | −66 | −1890 | 1839 | −442 | −957 | −36 | −1499 | 1204 | −132 | 616 | −382 | −1469 | −2048 | −1383 | 70 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 66(C) | 605 | 1553 | 739 | −17 | −1374 | −1488 | −182 | 260 | 969 | −203 | −397 | −263 | −1573 | 159 | 691 | −426 | −331 | −761 | −1567 | −1032 | 71 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 67(A) | 2327 | −956 | −3193 | −2728 | −1289 | −2677 | −2114 | 1664 | −2485 | −601 | −288 | −2403 | −2839 | −2263 | −2523 | −1871 | −1126 | 1617 | −2143 | −1765 | 72 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 68(K) | −532 | −1656 | −490 | 1321 | −1891 | −1527 | −172 | −124 | 2206 | −1591 | −782 | −223 | −1619 | 237 | −106 | −482 | −464 | −98 | −1904 | −1326 | 73 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 69(H) | 384 | −1854 | 936 | 889 | −2165 | −1363 | 1498 | −1909 | 1111 | −1866 | −948 | 1091 | −1464 | 421 | −131 | −284 | −342 | −69 | −2043 | −1364 | 74 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 70(G) | 1823 | −932 | −2330 | −2313 | −3120 | 2511 | −2158 | −2865 | −2331 | −3098 | −2209 | −1563 | −1912 | −2032 | −2419 | 1138 | −706 | −1883 | −3328 | −3077 | 75 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 71(V) | −1760 | −1333 | −4244 | −3789 | −1262 | −3902 | −3190 | 1495 | −3588 | 1270 | −96 | −3536 | −3677 | −3238 | −3534 | −3148 | −1725 | 2865 | −2654 | −2373 | 76 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 72(W) | −1054 | −2172 | −1112 | −403 | −2566 | −1917 | −286 | −2196 | 2516 | −2095 | −1292 | 1183 | −1958 | 140 | 1333 | −959 | −922 | −1867 | 2591 | −1720 | 77 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 73(D) | 611 | −1995 | 1525 | 937 | −2295 | −1400 | −148 | −2043 | 211 | −2006 | −1106 | −37 | −1553 | 1420 | −312 | −408 | 1235 | −1609 | −2193 | −1499 | 78 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 74(A) | 2716 | −902 | −2380 | −2205 | −2799 | −1197 | −1975 | −2459 | −2081 | −2736 | −1895 | −1520 | −1895 | −1844 | −2201 | 1191 | 1299 | −1669 | −3045 | −2758 | 79 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 75(G) | −1709 | −2833 | 2424 | −409 | −3781 | 2819 | −1457 | −3777 | −1728 | −3733 | −3076 | −739 | −2389 | −1180 | −2441 | −1557 | −1893 | −3158 | −3660 | −3038 | 80 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −212 | −2909 | −8150 | −273 | −2534 | −701 | −1378 | * | * | | | | | | | | | | | | |
| 76(A) | 2529 | −1119 | −2614 | −2330 | −1245 | −1983 | −1829 | −377 | −2042 | 1435 | −341 | −1937 | −2411 | −1873 | −2088 | −1266 | −1059 | −397 | −2063 | −1713 | 82 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 77(W) | −472 | −361 | −2421 | −1812 | −298 | −1979 | −826 | 1164 | −1486 | −143 | 2485 | 873 | −2028 | −1185 | −1426 | −1048 | −412 | 1116 | 2999 | −454 | 83 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 78(P) | −1198 | −1737 | −2187 | −2394 | −3665 | 2006 | −2550 | −3630 | −2743 | −3756 | −3008 | −2052 | 3474 | −2495 | −2835 | −1401 | −1593 | −2736 | −3511 | −3519 | 84 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 79(Q) | −999 | −1075 | −2106 | −1568 | −726 | −2370 | −1175 | 83 | −1185 | 1373 | 218 | −1566 | −2400 | 2445 | −1340 | −1445 | −946 | 1441 | −1501 | −1146 | 85 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 80(Q) | −885 | −779 | −2609 | −2018 | −481 | −2414 | −1253 | 1645 | −1736 | 799 | 1924 | −1827 | −2405 | 2262 | −1752 | −1484 | −821 | 802 | −1240 | −935 | 86 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 81(F) | −3342 | −2776 | −4026 | −4232 | 4354 | −3545 | −1431 | −2315 | −4038 | −1801 | −1900 | −3299 | −3780 | −3350 | −3645 | −3490 | −3420 | −2566 | −739 | 349 | 87 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 82(G) | −998 | −2100 | −120 | −175 | −2567 | 2528 | 2174 | −2558 | −587 | −2583 | −1806 | 1422 | −1966 | −461 | −1038 | −925 | −1088 | −2095 | −2657 | −1948 | 88 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 83(T) | −1213 | −1674 | −2755 | −2906 | −3163 | −1922 | −2659 | −2698 | −2788 | −3105 | −2612 | −2311 | −2600 | −2708 | −2753 | −1463 | 3819 | −2197 | −3286 | −3156 | 89 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 84(I) | −1286 | −1279 | −2907 | −2683 | −1446 | −2549 | −2198 | 3290 | −2407 | −726 | −534 | −2386 | 1172 | −2299 | −2437 | −1895 | −1392 | 283 | −2302 | −1913 | 90 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 85(T) | −493 | −1105 | −2189 | −2267 | −3101 | 1880 | −2196 | −2791 | −2334 | −3081 | −2269 | −1649 | −2058 | −2099 | −2410 | −719 | 3135 | −1948 | −3282 | −3046 | 91 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 86(V) | −1750 | −1296 | −4319 | −3957 | −1765 | −4038 | −3733 | 2364 | −3826 | −619 | −543 | −3716 | −3869 | −3685 | −3902 | −3354 | −1743 | 3012 | −3265 | −2817 | 92 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 87(S) | 923 | −962 | −2348 | −2422 | −3132 | −1207 | −2248 | −2850 | −2440 | −3140 | −2285 | −1624 | −1954 | −2158 | −2477 | 3171 | −758 | −1896 | −3362 | −3103 | 93 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 88(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 94 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 89(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 95 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 90(I) | −1880 | −1493 | −4193 | −3724 | −953 | −3837 | −2980 | 3251 | −3420 | 257 | 2372 | −3485 | −3608 | −3005 | −3310 | −3087 | −1840 | 617 | −2373 | −2155 | 96 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 91(S) | 2150 | −939 | −2407 | −2415 | −3075 | −1197 | −2205 | −2781 | −2384 | −3065 | −2205 | −1613 | −1936 | −2105 | −2436 | 2652 | −729 | −1850 | −3306 | −3049 | 97 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 92(M) | −979 | −1455 | −1242 | −1122 | −1434 | −1860 | −1131 | −1171 | −974 | −1285 | 4091 | 2176 | −2226 | −1017 | −1187 | −1166 | −1086 | −1063 | −1929 | −1345 | 98 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 93(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 99 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 94(T) | −959 | −1691 | −1249 | −949 | −2563 | −1747 | −929 | −2093 | 1282 | −2263 | −1554 | −995 | −2115 | −600 | −354 | −1037 | 3152 | −1726 | −2494 | −2098 | 100 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 95(E) | −572 | −1860 | −208 | 2213 | −2107 | −1461 | −191 | −1808 | 199 | −116 | −983 | −127 | 318 | 1199 | −269 | −475 | −517 | −1448 | −2078 | −1441 | 101 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 96(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 102 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 97(M) | −2406 | −2296 | −3638 | −3594 | −1525 | −3105 | −2824 | −1047 | −3121 | −596 | 5043 | −3293 | −3425 | −3046 | −2996 | −2911 | −2552 | −1398 | −2513 | −2207 | 103 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 98(R) | −2097 | −2786 | −2688 | −1415 | −3622 | −2625 | −555 | −2964 | 2585 | −2627 | −1957 | −1318 | −2577 | −137 | 3015 | −1979 | −1791 | −2732 | −2469 | −2363 | 104 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 99(Y) | −3615 | −2706 | −4169 | −4413 | 2626 | −4044 | −396 | −2535 | −3993 | −1939 | −1985 | −2747 | −3930 | −2852 | −3446 | −3296 | −3494 | −2686 | 347 | 4252 | 105 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 100(S) | −897 | −1462 | −2333 | −2543 | −3185 | −1640 | −2474 | −3294 | −2686 | −3497 | −2780 | −1973 | −2360 | −2483 | −2703 | 3465 | −1316 | −2413 | −3310 | −3025 | 106 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 101(L) | −2871 | −2457 | −4231 | −4103 | −1033 | −3803 | −3165 | −541 | −3734 | 3130 | −31 | −3935 | −3797 | −3286 | −3484 | −3713 | −2869 | −1136 | −2394 | −2220 | 107 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 102(V) | −1381 | −1065 | −3714 | −3252 | −1453 | −3300 | −2646 | 1872 | −3023 | −615 | −373 | −2949 | −3287 | −2816 | −3039 | −2506 | 1346 | 2750 | −2489 | −2087 | 108 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 103(S) | −897 | −1462 | −2333 | −2543 | −3185 | −1640 | −2474 | −3294 | −2686 | −3497 | −2780 | −1973 | −2360 | −2483 | −2703 | 3465 | −1316 | −2413 | −3310 | −3025 | 109 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 104(R) | −2957 | −3022 | −3318 | −2735 | −3796 | −2998 | −1968 | −3912 | −846 | −3631 | −3157 | −2611 | −3280 | −1724 | 4056 | −3026 | −2913 | −3650 | −3096 | −3185 | 110 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 105(E) | −1719 | −3572 | 2596 | 2779 | −3767 | −1632 | −993 | −3700 | −1241 | −3578 | −2920 | −234 | −2167 | −666 | −2090 | −1380 | −1789 | −3182 | −3742 | −2756 | 111 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 106(V) | −1746 | −1296 | −4308 | −3946 | −1757 | −4020 | −3712 | 2190 | −3811 | −614 | −539 | −3702 | −3858 | −3667 | −3884 | −3336 | −1740 | 3098 | −3250 | −2803 | 112 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 107(I) | −2091 | −1746 | −3971 | −3840 | −1676 | −3532 | −3289 | 3684 | −3581 | −659 | −693 | −3562 | −3674 | −3445 | −3521 | −3194 | −2146 | 449 | −2877 | −2493 | 113 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 108(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 114 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 109(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 115 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 110(S) | −352 | 2942 | −2955 | −2957 | −2876 | −1254 | −2382 | −2573 | −2692 | −2927 | −2128 | −1827 | −2001 | −2405 | −2607 | 3103 | −778 | −1757 | −3171 | −2911 | 116 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 111(I) | −2091 | −1746 | −3971 | −3840 | −1676 | −3532 | −3289 | 3684 | −3581 | −659 | −693 | −3562 | −3674 | −3445 | −3521 | −3194 | −2146 | 449 | −2877 | −2493 | 117 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 112(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 118 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 113(T) | 1556 | −936 | −2493 | −2457 | −2805 | −1256 | −2159 | −2210 | −2319 | −2681 | −1932 | −1656 | −1974 | −2089 | −2352 | −598 | 3235 | −1547 | −3111 | −2847 | 119 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 114(C) | 1784 | 2119 | −2013 | −1532 | −1093 | −1580 | −1089 | −436 | −1322 | −937 | −273 | 1093 | −1932 | −1127 | −1472 | −748 | −515 | 1585 | −1536 | −1163 | 120 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 115(M) | 1831 | 2019 | −2596 | −2038 | −605 | −1979 | −1126 | 244 | −1727 | −359 | 2501 | −1655 | −2145 | −1435 | −1683 | −1106 | −557 | 1087 | −1153 | −804 | 121 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 116(Q) | −987 | −2211 | −43 | −62 | −2833 | 2229 | −691 | −2616 | −407 | −2604 | −1797 | 1197 | −1917 | 2260 | −858 | −880 | −1045 | −2139 | −2772 | −2099 | 122 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 117(G) | 2313 | −1042 | −2391 | −2526 | −3250 | 2601 | −2372 | −2972 | −2637 | −3257 | −2407 | −1721 | −2032 | −2310 | −2646 | −662 | −859 | −2003 | −3434 | −3247 | 123 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 118(Q) | −914 | −2350 | −48 | 1661 | −2621 | −1571 | 2504 | −2400 | 68 | −2331 | −1486 | −201 | −1796 | 2646 | −351 | −754 | −865 | −1984 | −2463 | −1787 | 124 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 119(W) | −517 | −1294 | −733 | −183 | −1062 | −1605 | −234 | −1037 | 19 | −1207 | −456 | 1435 | −1690 | 33 | 756 | 411 | −454 | −819 | 3340 | 1286 | 125 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 120(M) | 410 | −469 | −2417 | −1828 | −341 | −2041 | −897 | 195 | −1513 | −156 | 3130 | −1534 | −2102 | −1230 | −1484 | −1117 | −507 | 954 | −894 | 2253 | 126 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 121(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 127 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 122(G) | 2142 | −930 | −2334 | −2298 | −3100 | 2237 | −2139 | −2842 | −2302 | −3074 | −2187 | −1557 | −1909 | −2010 | −2397 | 1136 | −701 | −1871 | −3308 | −3053 | 128 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 123(V) | −1514 | −1144 | −3950 | −3459 | 1821 | −3487 | −2577 | 2274 | −3208 | −209 | −87 | −3112 | −3362 | −2864 | −3118 | −2680 | −1476 | 2426 | −2194 | −1786 | 129 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 124(V) | −1743 | −1294 | −4292 | −3873 | −1511 | −3988 | −3433 | 2287 | −3712 | 598 | −319 | −3626 | −3774 | −3456 | −3716 | −3260 | −1717 | 2790 | −2931 | −2577 | 130 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 125(A) | 2911 | −954 | −2808 | −2665 | −2115 | −1577 | −2196 | −575 | −2445 | −1646 | −1202 | −1906 | −2208 | −2218 | −2451 | −901 | −876 | 1294 | −2727 | −2394 | 131 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 126(I) | −1764 | −1323 | −4298 | −3936 | −1668 | −3994 | −3655 | 3337 | −3783 | −508 | −462 | −3689 | −3838 | −3608 | −3835 | −3311 | −1759 | 1847 | −3164 | −2747 | 132 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 127(G) | −1157 | −1705 | −2169 | −2375 | −3654 | 3021 | −2534 | −3611 | −2730 | −3741 | −2984 | −2024 | 2418 | −2475 | −2826 | −1361 | −1555 | −2705 | −3513 | −3509 | 133 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 128(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 134 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 129(C) | −2476 | 5735 | −4102 | −4358 | −3712 | −2763 | −3545 | −3518 | −4167 | −3859 | −3569 | −3631 | −3363 | −4030 | −3832 | −2793 | −2860 | −3158 | −3464 | −3718 | 135 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 130(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 136 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 131(K) | −2620 | −2961 | −2461 | −2046 | −3743 | −2791 | −1570 | −3603 | 3784 | −3387 | −2839 | −2048 | −3039 | −1260 | −465 | −2604 | −2536 | −3331 | −3001 | −2988 | 137 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 132(N) | −2171 | −2655 | −1458 | −1748 | −3334 | −2364 | −2267 | −3943 | −2365 | −3936 | −3437 | 4205 | −2932 | −2205 | −2608 | −2224 | −2439 | −3392 | −3253 | −2909 | 138 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 133(M) | −2406 | −2296 | −3638 | −3594 | −1525 | −3105 | −2824 | −1047 | −3121 | −596 | 5043 | −3293 | −3425 | −3046 | −2996 | −2911 | −2552 | −1398 | −2513 | −2207 | 139 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 134(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 140 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 135(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 141 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 136(A) | 2180 | −935 | −2286 | −2196 | −3057 | 1098 | −2058 | −2796 | −2174 | −3021 | −2134 | −1516 | −1898 | −1906 | −2302 | 2146 | −689 | −1849 | −3256 | −2983 | 142 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 137(M) | −1799 | −1433 | −4142 | −3579 | −669 | −3668 | −2608 | 1558 | −3293 | 1235 | 3799 | −3296 | −3401 | −2717 | −3088 | −2843 | −1726 | 1156 | −2002 | −1868 | 143 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 138(I) | −2091 | −1746 | −3971 | −3840 | −1676 | −3532 | −3289 | 3684 | −3581 | −659 | −693 | −3562 | −3674 | −3445 | −3521 | −3194 | −2146 | 449 | −2877 | −2493 | 144 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 139(A) | 3103 | −1036 | −2445 | −2572 | −3222 | 1051 | −2380 | −2930 | −2650 | −3226 | −2381 | −1739 | −2034 | −2327 | −2648 | −664 | −857 | −1981 | −3412 | −3228 | 145 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 140(M) | −2325 | −1891 | −4598 | −4012 | −498 | −4222 | −3013 | 1242 | −3722 | 1864 | 3929 | −3855 | −3711 | −2910 | −3414 | −3439 | −2215 | −299 | −2076 | −2098 | 146 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 141(A) | 3103 | −1036 | −2445 | −2572 | −3222 | 1051 | −2380 | −2930 | −2650 | −3226 | −2381 | −1739 | −2034 | −2327 | −2648 | −664 | −857 | −1981 | −3412 | −3228 | 147 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 142(R) | −1588 | −2442 | −1399 | −953 | −3069 | −2171 | −708 | −2795 | 373 | −2625 | −1916 | 1858 | −2357 | −324 | 3294 | −1520 | −1505 | −2453 | −2523 | −2186 | 148 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 143(M) | −1448 | −1256 | −3396 | −2819 | −474 | −3024 | −1923 | 175 | −2473 | 2225 | 2756 | −2574 | −2922 | −2063 | −2375 | −2153 | 952 | −151 | −1599 | −1410 | 149 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 144(N) | −1662 | −3306 | 2055 | 78 | −3621 | −1643 | −1040 | −3622 | −1272 | −3531 | −2870 | 3477 | −2182 | −724 | −2071 | −1371 | −1757 | −3092 | −3633 | −2700 | 150 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 145(I) | −1066 | −921 | −2828 | −2239 | −1041 | −2675 | −1601 | 2235 | −1668 | −455 | −92 | −2067 | −2692 | −1688 | 1701 | −1795 | −1024 | 1960 | −1771 | −1396 | 151 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 146(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 152 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 147(S) | 1568 | −940 | −2267 | −2192 | −3082 | 1101 | −2068 | −2826 | −2185 | −3049 | −2159 | −1515 | −1901 | −1915 | −2313 | 2603 | −694 | −1866 | −3279 | −3006 | 153 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 148(I) | −1880 | −1492 | −4195 | −3728 | −963 | −3841 | −2991 | 3272 | −3425 | 246 | 2277 | −3490 | −3613 | −3014 | −3317 | −3092 | −1841 | 628 | −2385 | −2163 | 154 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 149(F) | −2204 | −1797 | −3724 | −3473 | 3206 | −3383 | −628 | −1077 | −3092 | −746 | 3167 | −2502 | −3309 | −2372 | −2792 | −2535 | −2120 | −1245 | 28 | 2460 | 155 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 150(V) | 1265 | −1028 | −3200 | −2994 | −1833 | −2150 | −2480 | 417 | −2771 | −1122 | −818 | −2349 | −2640 | −2559 | −2766 | −1464 | −1118 | 3028 | −2700 | −2325 | 156 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 151(Y) | −3482 | −2868 | −3701 | −3919 | 238 | −3552 | −1112 | −3000 | −3638 | −2516 | −2526 | −3027 | −3772 | −3101 | −3341 | −3418 | −3527 | −3071 | −441 | 4711 | 157 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 152(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 158 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 153(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 159 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 154(T) | −359 | −976 | −2225 | −2229 | −2900 | −1242 | −2074 | −2560 | −2170 | −2875 | −2064 | −1561 | −1958 | −1969 | −2247 | 1110 | 3375 | −1760 | −3152 | −2850 | 160 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 155(I) | −2091 | −1746 | −3971 | −3840 | −1676 | −3532 | −3289 | 3684 | −3581 | −659 | −693 | −3562 | −3674 | −3445 | −3521 | −3194 | −2146 | 449 | −2877 | −2493 | 161 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 156(H) | 861 | −1924 | −384 | 1010 | −2260 | −1477 | 1787 | −1974 | 1769 | −1918 | −1022 | −120 | −1566 | 362 | 697 | −417 | −459 | −1557 | −2073 | −1446 | 162 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 157(P) | −655 | −1502 | −711 | −557 | −2204 | −1463 | 2143 | −2122 | −586 | −2233 | −1445 | −688 | 2941 | −560 | −941 | 855 | −805 | −1657 | −2369 | −1763 | 163 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 158(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 164 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 159(H) | −744 | −2193 | −114 | 1118 | −2513 | −1512 | 2486 | −2252 | 1178 | −2183 | −1308 | 2230 | −1689 | 180 | −233 | −598 | −687 | −1823 | −2335 | −1670 | 165 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 160(W) | −2672 | −2139 | −3850 | −3748 | 941 | −3611 | −469 | −1691 | −3306 | 1047 | −1217 | −2551 | −3534 | −2514 | −2960 | −2788 | −2577 | −1799 | 4205 | 3466 | 166 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 161(K) | 386 | −1981 | 779 | 279 | −2295 | −1403 | −114 | −2043 | 2059 | −1991 | −1082 | 941 | −1536 | 1263 | −211 | −384 | −457 | −1602 | −2161 | −1476 | 167 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 162(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 168 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 163(K) | −1144 | −2365 | −912 | 2048 | −2856 | −1912 | −326 | −2459 | 2267 | −2295 | −1482 | −556 | −1989 | 108 | 1334 | −1013 | −1014 | −2093 | −2324 | −1881 | 169 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 164(D) | −1091 | −2610 | 2941 | 174 | −2957 | −1527 | −595 | −2750 | 1084 | −2696 | −1877 | −176 | −1885 | −206 | −1006 | 740 | −1098 | −2288 | −2880 | −2105 | 170 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 165(L) | −2387 | −1922 | −4674 | −4155 | −617 | −4366 | −3250 | 1889 | −3865 | 2650 | 558 | −4023 | −3847 | −3098 | −3586 | −3647 | −2296 | −38 | −2247 | −2224 | 171 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 166(N) | −1021 | −2427 | 1806 | 133 | −2870 | −1499 | −635 | −2647 | −521 | −2640 | −1825 | 2171 | −1874 | −255 | −1124 | −860 | 2122 | −2184 | −2853 | −2090 | 172 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 167(I) | −1830 | −1390 | −4327 | −3873 | −1210 | −3994 | −3274 | 2967 | −3678 | 1259 | −30 | −3633 | −3730 | −3283 | −3604 | −3249 | −1791 | 1570 | −2661 | −2417 | 173 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 168(V) | −1771 | −1603 | −3750 | −3689 | −2037 | −3050 | −3231 | 403 | −3479 | −1154 | −1076 | −3246 | −3399 | −3383 | −3437 | −2628 | −1917 | 3536 | −3074 | −2677 | 174 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 169(S) | −897 | −1462 | −2333 | −2543 | −3185 | −1640 | −2474 | −3294 | −2686 | −3497 | −2780 | −1973 | −2360 | −2483 | −2703 | 3465 | −1316 | −2413 | −3310 | −3025 | 175 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 170(A) | 2440 | −824 | −2371 | −2082 | −1993 | −1344 | −1704 | −1264 | −1899 | −1832 | −1137 | −1517 | −1946 | −1674 | −2005 | 1075 | −641 | 1474 | −2390 | −2055 | 176 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 171(F) | −3342 | −2776 | −4026 | −4232 | 4354 | −3545 | −1431 | −2315 | −4038 | −1801 | −1900 | −3299 | −3780 | −3350 | −3645 | −3490 | −3420 | −2566 | −739 | 349 | 177 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 172(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 178 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 173(A) | 2966 | −1031 | −2429 | −2551 | −3222 | 1544 | −2368 | −2934 | −2633 | −3225 | −2377 | −1727 | −2028 | −2309 | −2637 | −656 | −850 | −1980 | −3412 | −3224 | 179 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 174(V) | −1769 | −1342 | −4255 | −3793 | −1216 | −3901 | −3162 | 1633 | −3589 | 1486 | −51 | −3537 | −3667 | −3214 | −3518 | −3143 | −1731 | 2692 | −2609 | −2345 | 180 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 175(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 181 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 176(Q) | −729 | −2116 | −413 | 1096 | −2484 | −1587 | 1599 | −2186 | 1695 | −2094 | −1219 | −223 | −1698 | 2418 | 90 | −599 | −649 | −1770 | −2213 | −1615 | 182 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 177(W) | −1652 | −1707 | −2340 | −1879 | 1996 | −2733 | 2013 | −1398 | 1758 | −1386 | −938 | −1641 | −2751 | −1364 | −1762 | −1780 | −1577 | −1325 | 3577 | 2136 | 183 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 178(T) | −421 | −753 | −1251 | −704 | −846 | −1670 | −535 | 894 | −548 | −690 | −1 | 1376 | −1791 | −421 | −846 | 373 | 1461 | 858 | −1236 | −812 | 184 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 179(H) | 1498 | −1593 | −504 | 15 | −1895 | −1484 | 2279 | −1559 | 1119 | −1640 | −810 | −242 | −1611 | 194 | −171 | −462 | 815 | −1231 | −1914 | −1340 | 185 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 180(G) | −1515 | −2130 | −1298 | −1450 | −2658 | 3285 | 2212 | −3276 | −1691 | −3291 | −2638 | −1524 | −2562 | −1662 | −1925 | −1600 | −1764 | −2713 | −2804 | −2234 | 186 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 181(K) | −528 | −2010 | 1346 | 1082 | −2329 | −1408 | −118 | −2080 | 1475 | −2018 | −1108 | 1161 | −1543 | 331 | 1052 | −394 | −471 | −1632 | −2181 | −1494 | 187 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 182(M) | −1894 | −1521 | −4170 | −3679 | −840 | −3793 | −2866 | 2827 | −3360 | 375 | 3445 | −3437 | −3555 | −2902 | −3223 | −3028 | −1846 | 470 | −2249 | −2059 | 188 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 183(T) | −670 | −1758 | 1731 | −141 | −2591 | −1399 | −691 | −2319 | −499 | −2384 | −1543 | −387 | −1786 | −316 | −1016 | 1576 | 2044 | −1811 | −2624 | −1981 | 189 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 184(E) | 345 | −2074 | 925 | 1994 | −2378 | −1408 | −177 | −2135 | 922 | −2084 | −1183 | −38 | 641 | 264 | −356 | −444 | −536 | −1690 | −2261 | −1556 | 190 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 185(E) | −1493 | −2900 | 93 | 3174 | −2903 | −1743 | 1987 | −3042 | −646 | −2957 | −2238 | −411 | −2146 | −506 | −1121 | −1272 | −1503 | −2629 | −2905 | −2134 | 191 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 186(D) | −1293 | −2959 | 2673 | 2121 | −3219 | −1546 | −713 | −3043 | −707 | −2974 | −2191 | −158 | −1967 | −342 | −1394 | −1043 | 701 | −2567 | −3172 | −2311 | 192 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 187(F) | −1137 | −905 | −3250 | −2707 | 2365 | −2647 | −1016 | −34 | −2336 | 1239 | 267 | −2150 | −2626 | −1861 | −2133 | −1752 | −1069 | 1461 | −599 | 1844 | 193 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 188(K) | −479 | −1713 | −409 | 1031 | −1925 | −1467 | 1755 | −1650 | 1844 | −349 | −827 | −140 | −1556 | 319 | −75 | 403 | −411 | −1301 | −1900 | 843 | 194 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 189(G) | 433 | −2144 | 52 | 1047 | −2717 | 2303 | −615 | −2467 | −442 | −2482 | −1655 | 1123 | −1828 | −233 | −995 | −763 | −923 | −2000 | −2710 | −2005 | 195 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 190(V) | −1752 | −1320 | −4254 | −3806 | −1311 | −3916 | −3232 | 1701 | −3614 | 1188 | −140 | −3551 | −3693 | −3280 | −3568 | −3166 | −1718 | 2833 | −2703 | −2409 | 196 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 191(E) | −1199 | −1750 | −734 | 2668 | −1820 | −2038 | −1068 | 1892 | −867 | −1273 | −897 | −922 | −2295 | −797 | −1238 | −1340 | −1197 | −426 | −2325 | −1789 | 197 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 192(C) | −1182 | 3528 | −1398 | −620 | −2541 | −2038 | −358 | −2093 | 1181 | −2037 | −1272 | −747 | −2070 | 1553 | 2213 | −1123 | −1038 | −1817 | −2142 | −1774 | 198 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 193(N) | −1478 | −2527 | −261 | −403 | −2011 | −1837 | 2032 | −2925 | −735 | −2845 | −2195 | 3635 | −2259 | −721 | −1085 | −1352 | −1546 | −2522 | −2307 | −1431 | 199 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 194(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 200 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 195(C) | −1220 | 4911 | −3609 | −3314 | −1440 | −2525 | −2482 | 1565 | −2922 | −706 | −544 | −2678 | −2896 | −2710 | −2836 | −1869 | −1375 | 379 | −2371 | −1957 | 201 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 196(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 202 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 197(G) | −477 | −1115 | −1983 | −2189 | −3315 | 3154 | −2272 | −3172 | −2506 | −3387 | −2522 | −1599 | −2042 | −2177 | −2583 | 1217 | −905 | −2130 | −3477 | −3225 | 203 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 198(A) | 1653 | −1347 | −705 | −249 | −1969 | −1385 | −477 | −1629 | −159 | −1759 | −935 | −434 | 1285 | 1404 | −585 | −450 | 1019 | −1243 | −2070 | −1522 | 204 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 199(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 205 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 200(S) | 1870 | −938 | −2270 | −2183 | −3068 | 1488 | −2056 | −2810 | −2168 | −3032 | −2144 | −1511 | −1898 | −1901 | −2300 | 2236 | −690 | −1857 | −3265 | −2990 | 206 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 201(C) | −2476 | 5735 | −4102 | −4358 | −3712 | −2763 | −3545 | −3518 | −4167 | −3859 | −3569 | −3631 | −3363 | −4030 | −3832 | −2793 | −2860 | −3158 | −3464 | −3718 | 207 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 202(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | 4222 | 208 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 203(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3658 | −4222 | 209 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 204(M) | −2406 | −2296 | −3638 | −3594 | −1525 | −3105 | −2824 | −1047 | −3121 | −596 | 5043 | −3293 | −3425 | −3046 | −2996 | −2911 | −2552 | −1398 | −2513 | −2207 | 210 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 205(Y) | −3590 | −2700 | −4146 | −4379 | 2092 | −4028 | −404 | −2517 | −3963 | −1928 | −1973 | −2744 | −3921 | −2845 | −3431 | −3284 | −3474 | −2669 | 336 | 4423 | 211 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 206(T) | −1213 | −1674 | −2755 | −2906 | −3163 | −1922 | −2659 | −2698 | −2788 | −3105 | −2612 | −2311 | −2600 | −2708 | −2753 | −1463 | 3819 | −2197 | −3286 | −3156 | 212 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 207(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 213 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 208(N) | −2171 | −2655 | −1458 | −1748 | −3334 | −2364 | −2267 | −3943 | −2365 | −3936 | −3437 | 4205 | −2932 | −2205 | −2608 | −2224 | −2439 | −3392 | −3253 | −2909 | 214 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 209(T) | −1213 | −1674 | −2755 | −2906 | −3163 | −1922 | −2659 | −2698 | −2788 | −3105 | −2612 | −2311 | −2600 | −2708 | −2753 | −1463 | 3819 | −2197 | −3286 | −3156 | 215 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 210(M) | −2355 | −1988 | −4343 | −3834 | −504 | −4051 | −2868 | 105 | −3385 | 1451 | 4460 | −3680 | −3671 | −2806 | −3171 | −3327 | −2274 | −474 | −2039 | −1925 | 216 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 211(S) | 2150 | −939 | −2407 | −2415 | −3075 | −1197 | −2205 | −2781 | −2384 | −3065 | −2205 | −1613 | −1936 | −2105 | −2436 | 2652 | −729 | −1850 | −3306 | −3049 | 217 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 212(S) | −344 | −979 | −2190 | −2162 | −2959 | −1227 | −2042 | −2651 | −2116 | −2934 | −2100 | −1526 | −1941 | −1909 | −2222 | 2940 | 1775 | −1804 | −3187 | −2882 | 218 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 213(A) | 3048 | −932 | −2480 | −2533 | −3075 | −1200 | −2274 | −2765 | −2501 | −3071 | −2221 | −1658 | −1948 | −2205 | −2512 | 1225 | −739 | −1842 | −3322 | −3078 | 219 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 214(I) | −1924 | −1546 | −4067 | −3658 | 2312 | −3663 | −2081 | 3030 | −3367 | 150 | 99 | −3197 | −3492 | −2821 | −3179 | −2894 | −1877 | 293 | −1445 | −692 | 220 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 215(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 221 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 216(A) | 2389 | −814 | −2506 | −2162 | −1696 | −1545 | −1698 | −499 | −1942 | −1398 | −813 | −1640 | −2076 | −1723 | −2027 | −806 | 1148 | 1559 | −2200 | −1856 | 222 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 217(M) | −2576 | −2118 | −4725 | −4165 | −461 | −4430 | −3165 | 99 | −3811 | 2513 | 3454 | −4075 | −3839 | −2978 | −3488 | −3704 | −2457 | −591 | −2111 | −2145 | 223 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 218(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 224 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 219(M) | −2313 | −1968 | −4258 | −3765 | −518 | −3966 | −2806 | 98 | −3289 | 1292 | 4523 | −3599 | −3636 | −2769 | −3097 | −3249 | −2243 | −457 | −2026 | −1874 | 225 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 220(S) | −897 | −1462 | −2333 | −2543 | −3185 | −1540 | −2474 | −3294 | −2686 | −3497 | −2780 | −1973 | −2360 | −2483 | −2703 | 3455 | −1316 | −2413 | −3310 | −3025 | 226 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 221(L) | −2631 | −2159 | −4786 | −4228 | −462 | −4506 | −3231 | 96 | −3878 | 2828 | 2482 | −4157 | −3880 | −3016 | −3541 | −3793 | −2509 | −608 | −2134 | −2182 | 227 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 222(P) | −1501 | −1778 | −2473 | −2371 | −1710 | −2311 | −2045 | −1321 | −2060 | 827 | −1068 | −2173 | 3594 | −2082 | −2130 | −1799 | −1699 | −1373 | −2373 | −1942 | 228 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 223(Y) | −1068 | −1670 | −865 | −836 | −631 | 1198 | −767 | −1828 | −1059 | −1914 | −1304 | 692 | −2203 | −906 | −1387 | −1136 | −1163 | −1566 | −1185 | 3670 | 229 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 224(S) | −897 | −1462 | −2333 | −2543 | −3185 | −1640 | −2474 | −3294 | −2686 | −3497 | −2780 | −1973 | −2360 | −2483 | −2703 | 3465 | −1316 | −2413 | −3310 | −3025 | 230 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 225(S) | 1172 | −954 | −2367 | −2422 | −3120 | −1204 | −2237 | −2835 | −2426 | −3122 | −2265 | −1621 | −1948 | −2145 | −2467 | 3107 | −749 | −1884 | −3349 | −3092 | 231 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 226(S) | −342 | −975 | −2176 | −2124 | −2912 | −1229 | −2003 | −2594 | −2067 | −2878 | −2048 | −1510 | −1936 | −1866 | −2184 | 2553 | 2492 | −1773 | −3143 | −2833 | 232 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 227(M) | −720 | −1440 | −710 | −343 | −1228 | −1693 | 2436 | −1209 | −132 | −1364 | 3099 | 1904 | −1852 | −183 | −458 | −776 | −680 | −1004 | −1540 | −890 | 233 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 228(P) | 2240 | −1100 | −2241 | −2293 | −3037 | −1346 | −2188 | −2683 | −2317 | −2986 | −2210 | −1663 | 3041 | −2093 | −2391 | −722 | −895 | −1893 | −3243 | −2998 | 234 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 229(A) | 2958 | −1235 | −1299 | −1377 | −2868 | −1345 | −1673 | −2580 | −1661 | −2843 | −2054 | 1555 | −1995 | −1468 | −1921 | −715 | −888 | −1871 | −3064 | −2630 | 235 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 230(E) | −509 | −1046 | −884 | 1564 | −1116 | −1669 | −441 | −485 | −283 | 250 | −206 | −577 | 689 | −200 | −656 | −670 | −459 | 1290 | −1467 | −995 | 236 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 231(D) | −1203 | −2412 | 2595 | −117 | −3286 | −1536 | −1057 | −3176 | −1165 | −3186 | −2436 | −428 | −2068 | −736 | −1824 | 2377 | −1366 | −2578 | −3334 | −2552 | 237 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 232(Q) | 954 | −1983 | −100 | 971 | −2337 | 177 | −267 | −2067 | 81 | −2060 | −1189 | −125 | −1637 | 2600 | −418 | −514 | −597 | −1649 | −2268 | −1597 | 238 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 233(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 239 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 234(K) | −2620 | −2961 | −2461 | −2046 | −3743 | −2791 | −1570 | −3603 | 3784 | −3387 | −2839 | −2048 | −3039 | −1260 | −465 | −2604 | −2536 | −3331 | −3001 | −2988 | 240 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 235(R) | 377 | −1802 | −415 | 988 | −2095 | −1474 | −95 | −1786 | 1452 | −1785 | −911 | −135 | −1560 | 343 | 1555 | −409 | −431 | 376 | −1986 | −1375 | 241 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 236(D) | 1083 | −1565 | 2662 | −244 | −1941 | −1573 | −679 | 612 | −527 | −1651 | −980 | −490 | −1869 | −358 | −1003 | −771 | −766 | −903 | −2208 | −1633 | 242 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 237(E) | −1225 | −2868 | 1894 | 1948 | −3149 | −1532 | −671 | −2975 | −630 | −2902 | −2101 | −150 | −1935 | −293 | −1299 | 1884 | −1241 | −2496 | −3093 | −2248 | 243 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 238(C) | 1375 | 3262 | −2620 | −2108 | −827 | −1866 | −1267 | 1631 | −1811 | −599 | −10 | −1674 | −2137 | −1531 | −1786 | −1034 | 790 | 249 | −1361 | −1010 | 244 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 239(E) | 635 | −1796 | 1055 | 1761 | −2018 | −1464 | −263 | 1191 | 28 | −1767 | −946 | −148 | −1637 | 135 | −481 | −520 | −553 | −1300 | −2077 | −1441 | 245 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 240(E) | 593 | −2044 | −252 | 2548 | −2437 | −1542 | −329 | −2133 | 151 | −2120 | −1274 | −244 | −1738 | 89 | 946 | −646 | −717 | −1734 | −2305 | −1686 | 246 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 241(S) | 1884 | −835 | −1962 | −1576 | −1634 | −1436 | −1320 | 1041 | −1409 | −1453 | −781 | −1293 | −1922 | −1241 | −1606 | 1973 | −597 | −669 | −2036 | −1656 | 247 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 242(G) | 2267 | −1043 | −2388 | −2526 | −3253 | 2642 | −2373 | −2975 | −2639 | −3260 | −2410 | −1722 | −2033 | −2311 | −2648 | −663 | −860 | −2005 | −3436 | −3250 | 248 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 243(R) | −876 | −2087 | −829 | 1490 | −2474 | −1766 | −229 | −2106 | 1269 | −44 | −1198 | −424 | −1829 | 205 | 2225 | −775 | −768 | −1753 | −2143 | −1647 | 249 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 244(V) | 2339 | −967 | −2970 | −2766 | −1878 | −1847 | −2252 | 32 | −2541 | −1299 | −918 | −2087 | −2399 | −2316 | −2545 | −1157 | −971 | 2345 | −2605 | −2251 | 250 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 245(I) | −1827 | −1398 | −4307 | −3831 | −1099 | −3939 | −3142 | 2286 | −3619 | 1835 | 69 | −3579 | −3671 | −3177 | −3511 | −3178 | −1781 | 1918 | −2524 | −2310 | 251 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 246(V) | −1178 | −1448 | −1943 | −1452 | −1776 | −2261 | −1140 | −227 | 1866 | −1260 | −816 | −1444 | −2448 | −902 | −540 | −1496 | −1176 | 2697 | −2161 | −1764 | 252 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 247(E) | −508 | −1976 | 840 | 1547 | −2280 | −1393 | −117 | −2029 | 1400 | −1984 | −1077 | 1158 | −1531 | 330 | −253 | −378 | −454 | 262 | −2163 | −1471 | 253 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 248(M) | 1703 | −991 | −2901 | −2342 | −528 | −2567 | −1550 | 166 | −2031 | 1544 | 2668 | −2104 | −2591 | −1715 | −2010 | −1685 | −1052 | −12 | −1442 | −1177 | 254 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 249(I) | −1947 | −1516 | −4385 | −3885 | −916 | −4013 | −3118 | 2193 | −3656 | 2186 | 257 | −3656 | −3687 | −3109 | −3494 | −3250 | −1889 | 1383 | −2397 | −2258 | 255 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 250(E) | −1322 | −2647 | −272 | 2491 | −3071 | −1811 | −576 | −2759 | 2306 | −2633 | −1854 | −464 | −2066 | −175 | −177 | −1144 | −1256 | −2368 | −2692 | −2140 | 256 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 251(K) | −1395 | −2059 | −1711 | −1014 | −2215 | −2218 | −641 | −1709 | 3021 | −1652 | 2578 | −1075 | −2303 | −282 | 287 | −1423 | −1283 | −1603 | −2159 | −1803 | 257 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 252(D) | −1285 | −2888 | 2677 | 176 | −3210 | 1189 | −737 | −3047 | −715 | −2977 | −2195 | −190 | −1979 | 2106 | −1379 | −1050 | −1315 | −2564 | −3161 | −2320 | 258 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 253(I) | −2073 | −1632 | −4434 | −3975 | −911 | −4130 | −3238 | 3164 | −3706 | 1451 | 244 | −3779 | −3785 | −3187 | −3557 | −3413 | −2021 | 546 | −2449 | −2273 | 259 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 254(K) | −1570 | −2144 | −1887 | −1191 | −2098 | −2363 | −750 | −1603 | 3034 | 938 | −1112 | −1231 | −2436 | −408 | 215 | −1616 | −1443 | −1580 | −2166 | −1804 | 260 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 255(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 261 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 256(R) | −928 | −1705 | −1507 | −1055 | −2761 | −1730 | −896 | −2490 | −44 | −2489 | −1723 | −1042 | −2102 | −543 | 2614 | 2258 | −1053 | −1998 | −2546 | −2158 | 262 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 257(D) | −1280 | −2865 | 3154 | 175 | −3194 | −1547 | −743 | −3034 | −728 | −2971 | −2194 | −190 | −1979 | 1342 | −1391 | 553 | −1316 | −2552 | −3161 | −2317 | 263 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 258(I) | −1997 | −1562 | −4355 | −3927 | −1042 | −4066 | −3261 | 3343 | −3654 | 937 | 97 | −3718 | −3783 | −3239 | −3555 | −3354 | −1959 | 702 | −2549 | −2295 | 264 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 259(M) | −2252 | −1821 | −4572 | −3991 | −530 | −4164 | −2990 | 2068 | −3709 | 1993 | 3197 | −3808 | −3685 | −2916 | −3406 | −3378 | −2149 | −172 | −2084 | −2091 | 265 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 260(T) | −1213 | −1674 | −2755 | −2906 | −3163 | −1922 | −2659 | −2698 | −2788 | −3105 | −2612 | −2311 | −2600 | −2708 | −2753 | −1463 | 3819 | −2197 | −3286 | −3156 | 266 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 261(R) | −2131 | −2786 | −2704 | −1460 | −3618 | −2638 | −587 | −2976 | 1735 | −2645 | −1985 | −1353 | −2603 | −173 | 3492 | −2020 | −1828 | −2748 | −2484 | −2384 | 267 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 262(K) | −1349 | −2635 | −381 | 2083 | −3083 | −1857 | −565 | −2750 | 2690 | −2612 | −1837 | −514 | −2090 | −161 | −61 | −1178 | −1271 | −2369 | −2655 | −2138 | 268 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 263(A) | 2821 | −932 | −2451 | −2472 | −3065 | −1198 | −2233 | −2763 | −2434 | −3056 | −2201 | −1633 | −1940 | −2147 | −2468 | 1831 | −730 | −1840 | −3305 | −3055 | 269 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 264(F) | −2063 | −1686 | −4037 | −3677 | 3437 | −3644 | −1706 | 2063 | −3359 | 135 | 67 | −3095 | −3486 | −2739 | −3127 | −2876 | −2012 | −83 | −1038 | −158 | 270 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 265(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 271 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 266(N) | −1662 | −3306 | 2055 | 78 | −3621 | −1643 | −1040 | −3622 | −1272 | −3531 | −2870 | 3477 | −2182 | −724 | −2071 | −1371 | −1757 | −3092 | −3633 | −2700 | 272 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 267(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 273 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 268(I) | −1760 | −1307 | −4325 | −3962 | −1735 | −4042 | −3726 | 3135 | −3828 | −579 | −515 | −3722 | −3869 | −3673 | −3896 | −3359 | −1752 | 2276 | −3240 | −2806 | 274 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 269(T) | 1428 | −904 | −2334 | −2158 | −2747 | −1206 | −1940 | −2392 | −2037 | −2678 | −1846 | −1504 | −1896 | −1809 | −2163 | 902 | 3001 | −1635 | −2999 | −2705 | 275 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 270(V) | −1745 | −1300 | −4286 | −3858 | −1446 | −3967 | −3370 | 2358 | −3688 | 852 | −261 | −3606 | −3749 | −3403 | −3673 | −3232 | −1717 | 2643 | −2856 | −2524 | 276 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 271(V) | −1404 | −1072 | −3766 | −3305 | −1464 | −3356 | −2696 | 2276 | −3080 | −616 | −379 | −3001 | −3325 | −2870 | −3091 | −2563 | 1344 | 2521 | −2516 | −2113 | 277 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 272(M) | 866 | −1113 | −2656 | −2412 | −1322 | −1920 | −1883 | −487 | −2061 | −587 | 4451 | −1950 | −2387 | −1928 | −2078 | −1220 | −1053 | −498 | −2134 | −1803 | 278 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 273(A) | 2601 | −957 | −2898 | −2711 | −1943 | −1740 | −2211 | −165 | −2487 | −1406 | −1001 | −2008 | −2320 | −2260 | −2494 | −1053 | −929 | 1990 | −2626 | −2279 | 279 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 274(L) | −1171 | −983 | −3266 | −2733 | −796 | −2795 | −1888 | 590 | −2418 | 2001 | 198 | −2418 | −2816 | −2106 | −2362 | −1944 | 965 | 1777 | −1724 | −1426 | 280 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 275(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 281 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 276(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 282 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 277(S) | −897 | −1462 | −2333 | −2543 | −3185 | −1640 | −2474 | −3294 | −2686 | −3497 | −2780 | −1973 | −2360 | −2483 | −2703 | 3465 | −1316 | −2413 | −3310 | −3025 | 283 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 278(T) | −1213 | −1674 | −2755 | −2906 | −3163 | −1922 | −2659 | −2698 | −2788 | −3105 | −2612 | −2311 | −2600 | −2708 | −2753 | −1463 | 3819 | −2197 | −3286 | −3156 | 284 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 279(N) | −2171 | −2655 | −1458 | −1748 | −3334 | −2364 | −2267 | −3943 | −2365 | −3936 | −3437 | 4205 | −2932 | −2205 | −2608 | −2224 | −2439 | −3392 | −3253 | −2909 | 285 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 280(A) | 3134 | −934 | −2491 | −2567 | −3083 | −1203 | −2300 | −2766 | −2540 | −3082 | −2237 | −1672 | −1954 | −2240 | −2537 | 874 | −747 | −1844 | −3333 | −3093 | 286 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 281(V) | −984 | −1045 | −3169 | −2909 | −1709 | −2304 | −2404 | 531 | −2643 | −988 | −697 | −2378 | −2722 | −2480 | −2661 | −1601 | 1504 | 3014 | −2588 | −2201 | 287 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 282(L) | −2631 | −2159 | −4786 | −4228 | −462 | −4506 | −3231 | 96 | −3878 | 2828 | 2482 | −4157 | −3880 | −3016 | −3541 | −3793 | −2509 | −608 | −2134 | −2182 | 288 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 283(H) | −3205 | −3079 | −2723 | −2890 | −2110 | −3046 | 5295 | −4135 | −2617 | −3813 | −3561 | −2886 | −3482 | −2833 | −2620 | −3291 | −3356 | −3895 | −2397 | −1681 | 289 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 284(L) | −1623 | −1338 | −3726 | −3164 | −251 | −3255 | −1820 | 1373 | −2808 | 2371 | 514 | −2785 | −3086 | −2281 | −2613 | −2389 | −1543 | −161 | −1311 | 1782 | 290 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 285(L) | −2333 | −1873 | −4640 | −4127 | −650 | −4326 | −3241 | 2176 | −3843 | 2519 | 523 | −3982 | −3833 | −3105 | −3579 | −3604 | −2247 | 56 | −2268 | −2230 | 291 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 286(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 292 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 287(M) | −1886 | −1507 | −4178 | −3693 | −877 | −3806 | −2901 | 3008 | −3380 | 335 | 3109 | −3451 | −3570 | −2934 | −3251 | −3044 | −1840 | 524 | −2288 | −2089 | 293 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 288(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 294 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 289(H) | −1490 | −2484 | −362 | −476 | −1816 | −1880 | 4320 | −2854 | −684 | −2770 | −2133 | 2185 | −2285 | −728 | −1000 | −1377 | −1550 | −2475 | −2146 | −1255 | 295 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 290(A) | 2439 | −911 | −2326 | −2131 | −2811 | −1197 | −1934 | −2480 | −2011 | −2745 | −1898 | −1490 | −1888 | −1785 | −2153 | 1898 | 1073 | −1682 | −3044 | −2749 | 296 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 291(I) | 2038 | −985 | −3388 | −2919 | −1320 | −2893 | −2277 | 2155 | −2677 | −587 | −297 | −2593 | −2992 | −2450 | −2697 | −2087 | −1208 | 1681 | −2229 | −1846 | 297 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 292(G) | −1243 | −2769 | 311 | 1902 | −3172 | 1980 | −744 | −2992 | −697 | −2936 | −2152 | 1923 | −1974 | −377 | −1331 | −1030 | −1284 | −2506 | −3125 | −2308 | 298 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 293(V) | −1738 | −1298 | −4281 | −3921 | −1737 | −3979 | −3665 | 1917 | −3774 | −601 | −528 | −3671 | −3834 | −3628 | −3843 | −3293 | −1735 | 3205 | −3215 | −2770 | 299 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 294(E) | −833 | −2344 | 1092 | 2412 | −2643 | −1464 | −386 | −2413 | −146 | −2369 | −1505 | −96 | 562 | 29 | −717 | −666 | 862 | −1966 | −2562 | −1818 | 300 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 295(W) | −1380 | −1116 | −3614 | −3026 | 1322 | −2981 | −1582 | 1966 | −2661 | 1775 | 556 | −2562 | −2865 | −2117 | −2424 | −2098 | −1302 | −187 | 2908 | −629 | 301 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 296(T) | −350 | −973 | −2204 | −2178 | −2893 | −1236 | −2035 | −2561 | −2117 | −2862 | −2043 | −1536 | −1946 | −1916 | −2214 | 1618 | 3198 | −1758 | −3137 | −2831 | 302 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 297(L) | −1443 | −1269 | −3144 | −2576 | −528 | −3014 | −1816 | 1945 | −2155 | 2102 | 508 | −2422 | −2899 | 1193 | −2133 | −2129 | −1369 | −50 | −1616 | −1384 | 303 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 298(D) | −1826 | −3682 | 3559 | 1199 | −3883 | −1662 | −1073 | −3846 | −1391 | −3720 | −3110 | −272 | −2222 | −760 | −2283 | −1471 | −1913 | −3321 | −3864 | −2864 | 304 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 299(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 305 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 300(F) | −3342 | −2776 | −4026 | −4232 | 4354 | −3545 | −1431 | −2315 | −4038 | −1801 | −1900 | −3299 | −3780 | −3350 | −3645 | −3490 | −3420 | −2566 | −739 | 349 | 306 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 301(Q) | −1048 | −2608 | 205 | 2170 | −2893 | −1535 | −505 | −2680 | −255 | −2604 | −1769 | 1814 | −1849 | 2272 | −789 | −848 | −1028 | −2228 | −2770 | −2013 | 307 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 302(R) | 1083 | −1687 | 691 | 135 | −2058 | −1406 | −178 | −1755 | 214 | −1793 | −924 | −145 | −1553 | 247 | 1670 | −383 | 1217 | −1367 | −2031 | −1404 | 308 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 303(I) | −1915 | −1536 | −4077 | −3667 | 2027 | −3678 | −2155 | 3137 | −3381 | 144 | 94 | −3225 | −3506 | −2848 | −3202 | −2914 | −1871 | 345 | −1522 | −791 | 309 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 304(R) | −689 | −2015 | −494 | 24 | −2395 | −1582 | −184 | −2087 | 444 | −2020 | −1151 | 1161 | −1687 | 1832 | 2131 | 626 | −614 | −1684 | −2156 | −1573 | 310 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 305(D) | 387 | −1967 | 1600 | 1359 | −2275 | −1391 | 1561 | −2025 | 282 | −1976 | −1067 | −25 | −1525 | 342 | 1024 | −369 | −443 | −1584 | −2152 | −1462 | 311 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 306(R) | −1460 | −2315 | −1793 | −887 | −2832 | −2237 | −431 | −2288 | 2193 | −2199 | −1473 | −946 | −2245 | −20 | 2706 | −1394 | −1275 | 591 | −2248 | −1961 | 312 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 307(V) | −941 | −1027 | −3099 | −2832 | −1692 | −2234 | −2324 | 470 | −2565 | −1003 | −695 | −2305 | −2663 | −2399 | −2587 | −1527 | 1858 | 2876 | −2536 | −2152 | 313 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 308(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 314 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 309(V) | −1090 | −1215 | −2097 | −1824 | −819 | −2221 | 2699 | −287 | −1392 | −1027 | −591 | −1674 | −2482 | −1446 | −1482 | −1482 | −1143 | 2879 | −1420 | −707 | 315 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 310(L) | −2439 | −1972 | −4702 | −4181 | −588 | −4401 | −3258 | 1582 | −3881 | 2757 | 587 | −4061 | −3862 | −3093 | −3590 | −3689 | −2344 | −130 | −2230 | −2217 | 316 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 311(C) | 2157 | 4166 | −3012 | −2973 | −2780 | 1022 | −2337 | −2398 | −2724 | −2744 | −1930 | −1786 | −1943 | −2372 | −2623 | −540 | −692 | −1624 | −3091 | −2881 | 317 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 312(D) | −1732 | −3453 | 3468 | 99 | −3733 | −1645 | −1066 | −3747 | −1356 | −3641 | −3008 | 1690 | −2201 | −755 | −2209 | −1416 | −1833 | −3208 | −3752 | −2776 | 318 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 313(L) | −2477 | −2023 | −4713 | −4122 | 1592 | −4329 | −2920 | 72 | −3835 | 2593 | 2472 | −3948 | −3754 | −2914 | −3466 | −3550 | −2350 | −634 | −1927 | −1830 | 319 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 314(K) | −2620 | −2961 | −2461 | −2046 | −3743 | −2791 | −1570 | −3603 | 3784 | −3387 | −2839 | −2048 | −3039 | −1260 | −465 | −2604 | −2536 | −3331 | −3001 | −2988 | 320 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 315(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 321 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 316(S) | −897 | −1462 | −2333 | −2543 | −3185 | −1640 | −2474 | −3294 | −2686 | −3497 | −2780 | −1973 | −2360 | −2483 | −2703 | 3465 | −1316 | −2413 | −3310 | −3025 | 322 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 317(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 323 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 318(K) | 2 | −2257 | −1073 | −374 | −2740 | −1908 | −278 | −2339 | 2328 | −2192 | −1373 | −562 | −1953 | 2273 | 1344 | −952 | −933 | −1980 | −2234 | −1799 | 324 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 319(Y) | −3482 | −2868 | −3701 | −3919 | 238 | −3552 | −1112 | −3000 | −3638 | −2516 | −2526 | −3027 | −3772 | −3101 | −3341 | −3418 | −3527 | −3071 | −441 | 4711 | 325 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 320(M) | −1559 | −1267 | −3829 | −3380 | −1103 | −3357 | −2655 | 805 | −3067 | −64 | 3046 | −3065 | −3326 | −2779 | −3011 | −2591 | −1556 | 2855 | −2312 | −1998 | 326 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 321(M) | 1225 | −469 | −2256 | −1679 | 1656 | −1926 | −870 | 90 | −1396 | −210 | 2763 | −1424 | −2028 | −1129 | −1411 | −1008 | 712 | 154 | −951 | −586 | 327 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 322(T) | −738 | −2094 | −84 | 1704 | −2416 | −1495 | −317 | −2135 | 61 | −2127 | −1275 | −163 | −1704 | 1857 | −405 | −613 | 1930 | −1734 | −2331 | −1668 | 328 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 323(D) | −1746 | −3458 | 3540 | 90 | −3744 | −1650 | −1081 | −3767 | −1381 | −3662 | −3036 | 1386 | −2211 | −772 | −2239 | −1429 | −1850 | −3226 | −3765 | −2789 | 329 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 324(L) | −2451 | −1983 | −4707 | −4186 | −582 | −4409 | −3259 | 1510 | −3884 | 2778 | 592 | −4069 | −3865 | −3091 | −3590 | −3698 | −2355 | −150 | −2226 | −2214 | 330 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 325(H) | −2923 | −2573 | −2959 | −2926 | 826 | −3449 | 4553 | −2508 | −2463 | −2054 | −1948 | −2279 | −3499 | −2191 | −2397 | −2761 | −2855 | −2540 | 123 | 2920 | 331 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 326(K) | 373 | −1957 | −342 | 1025 | −2297 | −1472 | −98 | −2018 | 2111 | −1954 | −1056 | 906 | −1570 | 352 | 685 | −424 | −473 | −1592 | −2105 | −1469 | 332 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 327(V) | 1739 | −1008 | −3509 | −3043 | −1376 | −3028 | −2406 | 1765 | −2807 | −615 | −334 | −2718 | −3093 | −2585 | −2823 | −2226 | −1263 | 2376 | −2322 | −1931 | 333 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 328(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 334 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 329(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 335 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 330(I) | −1758 | −1302 | −4331 | −3970 | −1756 | −4054 | −3748 | 2976 | −3840 | −603 | −533 | −3731 | −3877 | −3693 | −3914 | −3372 | −1750 | 2505 | −3265 | −2824 | 336 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 331(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 337 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 332(Q) | 1795 | −1440 | −730 | −492 | −2453 | 682 | −812 | −2151 | −508 | −2256 | −1426 | −624 | −1796 | 2666 | −901 | −590 | −689 | −1636 | −2510 | −1971 | 338 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 333(V) | −1771 | −1603 | −3750 | −3689 | −2037 | −3050 | −3231 | 403 | −3479 | −1154 | −1076 | −3246 | −3399 | −3383 | −3437 | −2628 | −1917 | 3536 | −3074 | −2677 | 339 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 334(M) | −2355 | −1988 | −4343 | −3834 | −504 | −4051 | −2868 | 105 | −3385 | 1451 | 4460 | −3680 | −3671 | −2806 | −3171 | −3327 | −2274 | −474 | −2039 | −1925 | 340 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 335(K) | −2620 | −2961 | −2461 | −2046 | −3743 | −2791 | −1570 | −3603 | 3784 | −3387 | −2839 | −2048 | −3039 | −1260 | −465 | −2604 | −2536 | −3331 | −3001 | −2988 | 341 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 336(Y) | −1187 | −974 | −3186 | −2638 | −117 | −2732 | −1255 | 1905 | −2270 | 73 | 1977 | −2217 | −2699 | −1882 | −2144 | −1841 | −1124 | 71 | −907 | 3254 | 342 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 337(L) | −2871 | −2457 | −4231 | −4103 | −1033 | −3803 | −3165 | −541 | −3734 | 3130 | −31 | −3935 | −3797 | −3286 | −3484 | −3713 | −2869 | −1136 | −2394 | −2220 | 343 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 338(L) | −2871 | −2457 | −4231 | −4103 | −1033 | −3803 | −3165 | −541 | −3734 | 3130 | −31 | −3935 | −3797 | −3286 | −3484 | −3713 | −2869 | −1136 | −2394 | −2220 | 344 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 339(K) | −864 | −1785 | −860 | −366 | −2128 | −1763 | −407 | −1612 | 2624 | −1800 | −1045 | 629 | −1900 | −28 | 62 | −851 | −805 | 1127 | −2064 | −1581 | 345 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 340(N) | 602 | −1686 | −275 | 1008 | −1926 | −1415 | 1528 | −1618 | 244 | −1673 | −815 | 1897 | −1530 | 299 | −244 | −371 | −391 | 322 | −1934 | −1306 | 346 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 341(G) | −1709 | −2639 | 1362 | −690 | −3785 | 3257 | −1671 | −3805 | −1946 | −3792 | −3137 | −980 | −2480 | −1424 | −2576 | −1630 | −1936 | −3150 | −3628 | −3155 | 347 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 342(F) | −942 | −799 | −2828 | −2226 | 1797 | −2476 | −1269 | 1109 | 581 | 1793 | 516 | −1952 | −2453 | −1557 | −1815 | −1558 | −875 | 52 | −1138 | −794 | 348 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 343(L) | −2451 | −1983 | −4707 | −4186 | −582 | −4409 | −3259 | 1510 | −3884 | 2778 | 592 | −4069 | −3865 | −3091 | −3590 | −3698 | −2355 | −150 | −2226 | −2214 | 349 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 344(H) | −3205 | −3079 | −2723 | −2890 | −2110 | −3046 | 5295 | −4135 | −2617 | −3813 | −3561 | −2886 | −3482 | −2833 | −2620 | −3291 | −3356 | −3895 | −2397 | −1681 | 350 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 345(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 351 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 346(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 352 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 347(C) | 774 | 4452 | −2162 | −1688 | −1962 | −1478 | −1302 | −1474 | −944 | −1796 | −1088 | −1351 | −1979 | −1147 | 1684 | −732 | −719 | −1116 | −2225 | −1881 | 353 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 348(L) | −2387 | −1922 | −4674 | −4155 | −617 | −4366 | −3250 | 1889 | −3865 | 2650 | 558 | −4023 | −3847 | −3098 | −3586 | −3647 | −2296 | −38 | −2247 | −2224 | 354 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 349(T) | −1213 | −1674 | −2755 | −2906 | −3163 | −1922 | −2659 | −2698 | −2788 | −3105 | −2612 | −2311 | −2600 | −2708 | −2753 | −1463 | 3819 | −2197 | −3286 | −3156 | 355 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 350(C) | −1489 | 2972 | −4007 | −3563 | −1524 | −3541 | −2939 | 2612 | −3350 | −617 | −413 | −3224 | −3470 | −3129 | −3335 | −2770 | −1475 | 2269 | −2657 | −2248 | 356 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 351(T) | −364 | −979 | −2232 | −2250 | −2904 | −1245 | −2090 | −2559 | −2191 | −2881 | −2075 | −1571 | −1964 | −1991 | −2260 | 905 | 3428 | −1762 | −3159 | −2858 | 357 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 352(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 358 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 353(K) | −1716 | −2632 | −2004 | −1008 | −3336 | −2379 | −444 | −2764 | 2775 | −2484 | −1756 | −1035 | −2357 | 2151 | 1811 | −1592 | −1477 | −2481 | −2391 | −2172 | 359 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 354(T) | −1213 | −1674 | −2755 | −2906 | −3163 | −1922 | −2659 | −2698 | −2788 | −3105 | −2612 | −2311 | −2600 | −2708 | −2753 | −1463 | 3819 | −2197 | −3286 | −3156 | 360 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 355(V) | −1771 | −1339 | −4275 | −3816 | −1235 | −3919 | −3194 | 2139 | −3617 | 1520 | −66 | −3558 | −3681 | −3244 | −3547 | −3164 | −1733 | 2390 | −2634 | −2369 | 361 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 356(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 362 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 357(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 363 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 358(N) | −823 | −1917 | −96 | 1188 | −2187 | −1547 | −506 | −1711 | −265 | −1955 | −1191 | 2711 | −1815 | −144 | −747 | −757 | −815 | 1140 | −2297 | −1666 | 364 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 359(L) | −2153 | −1779 | −4360 | −3884 | −675 | −3965 | −3012 | 392 | −3561 | 2726 | 467 | −3673 | −3662 | −2955 | −3355 | −3239 | −2102 | 1281 | −2207 | −2099 | 365 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 360(E) | 1136 | −2084 | −175 | 2027 | −2436 | −1510 | −274 | −2147 | 1525 | −2118 | −1254 | −175 | −1692 | 152 | −251 | −593 | −670 | −1736 | −2296 | −1650 | 366 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 361(H) | 893 | −1761 | 1357 | 214 | −2092 | −1387 | 1862 | −1810 | 229 | −1825 | −942 | −83 | −1527 | 293 | −273 | 640 | 793 | −1409 | −2050 | −1397 | 367 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 362(I) | 608 | −458 | −2776 | −2176 | 1666 | −2202 | −1113 | 1712 | −1836 | −222 | 338 | −1782 | −2245 | −1512 | −1731 | −1292 | 867 | 1366 | −1036 | −684 | 368 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 363(P) | −922 | −1912 | 1681 | −141 | −2123 | −1604 | −687 | −1787 | −550 | 187 | −1245 | −427 | 2677 | −363 | −1049 | −882 | −947 | −1524 | −2338 | −1711 | 369 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 364(D) | −1692 | −3605 | 3364 | 1256 | −3770 | −1599 | −957 | −3700 | −1216 | −3569 | −2909 | 1025 | −2138 | −628 | −2083 | −1346 | −1761 | −3174 | −3765 | −2738 | 370 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 365(Q) | −877 | −1646 | −633 | 499 | −1610 | −1781 | −505 | −1210 | −63 | 1648 | −649 | −558 | −1931 | 2241 | −360 | −907 | −814 | −1097 | −1882 | −1385 | 371 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 366(P) | −648 | −2019 | 1139 | 203 | −2354 | −1436 | −285 | −2089 | 29 | −2086 | −1217 | −114 | 1965 | 1445 | −492 | −529 | 1244 | −1672 | −2300 | −1616 | 372 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −571 | −7108 | −1646 | −894 | −1115 | −701 | −1378 | * | * | | | | | | | | | | | | |
| 367(R) | −422 | −1009 | −851 | −304 | 1406 | −1496 | −183 | −740 | 147 | −894 | −230 | −440 | 775 | 21 | 2009 | −539 | −381 | −568 | −1136 | −521 | 373 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −23 | −6560 | −7602 | −894 | −1115 | −341 | −2249 | * | * | | | | | | | | | | | | |
| 368(D) | 1472 | −1668 | 1835 | −70 | −2356 | −1385 | −511 | −2062 | −246 | −2128 | −1275 | −318 | 1353 | −118 | −746 | −526 | 425 | −1602 | −2380 | −1752 | 374 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 369(G) | −1044 | −2230 | 2141 | −100 | −3222 | 2291 | −982 | −3045 | −1033 | −3050 | −2258 | −395 | −1985 | −644 | −1669 | 858 | −1207 | −2428 | −3250 | −2493 | 375 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 370(Q) | −2562 | −2904 | −1886 | −1971 | −3251 | −2661 | −2079 | −3690 | −1565 | −3469 | −3081 | −2107 | −3091 | 4371 | −1665 | −2585 | −2674 | −3411 | −3077 | −2821 | 376 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 371(D) | −1275 | −2955 | 2862 | 1330 | −3205 | −1556 | −670 | −3029 | 1509 | −2936 | −2141 | −158 | −1955 | −290 | −1213 | −1025 | −1281 | −2554 | −3111 | −2272 | 377 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 372(V) | −1738 | −1298 | −4281 | −3921 | −1737 | −3979 | −3665 | 1917 | −3774 | −601 | −528 | −3671 | −3834 | −3628 | −3843 | −3293 | −1735 | 3205 | −3215 | −2770 | 378 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 373(I) | −2091 | −1746 | −3971 | −3840 | −1676 | −3532 | −3289 | 3684 | −3581 | −659 | −693 | −3562 | −3674 | −3445 | −3521 | −3194 | −2146 | 449 | −2877 | −2493 | 379 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 374(M) | −584 | −1354 | −847 | −246 | −1467 | −1659 | 2505 | −1087 | 212 | −374 | 2571 | −449 | −1729 | 1171 | 1074 | −634 | −507 | −876 | −1617 | −1128 | 380 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 375(P) | −910 | −2031 | −73 | 1195 | −2792 | −1488 | −794 | −2539 | −629 | −2588 | −1788 | −401 | 3005 | −439 | −1131 | 612 | −1014 | −2050 | −2815 | −2151 | 381 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 376(W) | −1588 | −1300 | −3783 | −3197 | −329 | −3245 | −1926 | 2071 | −2827 | 1901 | 558 | −2822 | −3072 | −2297 | −2616 | −2381 | −1508 | −111 | 3483 | −1042 | 382 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 377(E) | −1024 | −2640 | 1844 | 2310 | −2908 | −1498 | −505 | −2711 | −344 | −2636 | −1791 | −107 | −1824 | 1521 | −957 | 207 | −1011 | −2243 | −2817 | −2021 | 383 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 378(N) | −826 | −2349 | 1089 | 227 | −2651 | −1487 | −341 | −2416 | 1494 | −2346 | −1475 | 2601 | −1724 | 1005 | −522 | −657 | −787 | −1968 | −2511 | −1791 | 384 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 379(P) | 1932 | −1116 | −2232 | −2301 | −3058 | −1358 | −2206 | −2706 | −2336 | −3009 | −2238 | −1674 | 3274 | −2114 | −2406 | −739 | −914 | −1913 | −3260 | −3019 | 385 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 380(V) | −914 | −773 | −2713 | −2129 | −712 | −2505 | −1388 | 1452 | 1084 | 1324 | 204 | −1926 | −2507 | −1580 | −1808 | −1591 | −859 | 1713 | −1424 | −1081 | 386 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 381(Y) | −1484 | −2331 | −1762 | −887 | −2436 | −2254 | −420 | −2325 | 2137 | −2195 | −1475 | −949 | −2258 | −39 | 1983 | −1411 | −1295 | −2075 | −2087 | 2868 | 387 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 382(E) | 1256 | −1890 | −206 | 1353 | −2196 | −1401 | −89 | −1930 | 812 | −1898 | −996 | −45 | 547 | 1252 | −162 | −356 | −414 | −1507 | −2083 | −1416 | 388 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 383(Q) | −752 | −2272 | 1586 | 1407 | −2561 | −1448 | −308 | −2329 | −23 | −2276 | −1396 | −71 | −1677 | 1749 | −577 | −590 | 1569 | −1881 | −2459 | −1727 | 389 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 384(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 390 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 385(H) | −964 | −2089 | −200 | −136 | −2264 | −1600 | 3833 | −2320 | −296 | −2338 | −1558 | 1362 | 1479 | −276 | −699 | −881 | −992 | −1924 | −2364 | −1652 | 391 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 386(L) | −2451 | −1983 | −4707 | −4186 | −582 | −4409 | −3259 | 1510 | −3884 | 2778 | 592 | −4069 | −3865 | −3091 | −3590 | −3698 | −2355 | −150 | −2226 | −2214 | 392 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 387(Q) | 1643 | −1017 | −1196 | −721 | −1189 | −1714 | −668 | 1336 | −497 | −907 | −297 | −823 | −1893 | 2044 | −794 | −784 | −569 | −339 | −1579 | −1135 | 393 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 388(I) | −1760 | −1308 | −4323 | −3961 | −1730 | −4039 | −3721 | 3156 | −3825 | −575 | −512 | −3720 | −3867 | −3669 | −3893 | −3356 | −1753 | 2241 | −3236 | −2802 | 394 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 389(L) | −2871 | −2457 | −4231 | −4103 | −1033 | −3803 | −3165 | −541 | −3734 | 3130 | −31 | −3935 | −3797 | −3286 | −3484 | −3713 | −2869 | −1136 | −2394 | −2220 | 395 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 390(K) | −1259 | −2115 | −1267 | −676 | −970 | −2105 | 1794 | −2040 | 2549 | −1955 | −1282 | −808 | −2165 | −167 | 114 | −1192 | −1140 | −1801 | −1301 | 2517 | 396 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 391(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | 4222 | 397 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 392(N) | −2171 | −2655 | −1458 | −1748 | −3334 | −2364 | −2267 | −3943 | −2365 | −3936 | −3437 | 4205 | −2932 | −2205 | −2608 | −2224 | −2439 | −3392 | −3253 | −2909 | 398 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 393(L) | −2871 | −2457 | −4231 | −4103 | −1033 | −3803 | −3165 | −541 | −3734 | 3130 | −31 | −3935 | −3797 | −3286 | −3484 | −3713 | −2869 | −1136 | −2394 | −2220 | 399 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 394(A) | 3121 | −934 | −2489 | −2561 | −3081 | −1203 | −2295 | −2766 | −2533 | −3080 | −2234 | −1669 | −1953 | −2234 | −2533 | 936 | −746 | −1844 | −3331 | −3090 | 400 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 395(E) | −522 | −1773 | −240 | 1676 | −2248 | −1396 | −289 | −1968 | 50 | −1989 | −1115 | −174 | 1198 | 131 | −448 | 1226 | 677 | −1538 | −2214 | −1565 | 401 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 396(E) | −1481 | −3230 | 1425 | 2936 | −3481 | 751 | −843 | −3354 | −954 | −3256 | −2520 | −187 | −2057 | −492 | −1711 | −1193 | −1527 | −2852 | −3445 | −2523 | 402 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 397(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | 4222 | 403 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 398(A) | 2847 | −932 | −2454 | −2477 | −3066 | −1198 | −2236 | −2763 | −2439 | −3057 | −2202 | −1635 | −1940 | −2152 | −2471 | 1777 | −731 | −1840 | −3306 | −3056 | 404 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 399(V) | −1771 | −1603 | −3750 | −3689 | −2037 | −3050 | −3231 | 403 | −3479 | −1154 | −1076 | −3246 | −3399 | −3383 | −3437 | −2628 | −1917 | 3536 | −3074 | −2677 | 405 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 400(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 406 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 401(K) | −2620 | −2961 | −2461 | −2046 | −3743 | −2791 | −1570 | −3603 | 3784 | −3387 | −2839 | −2048 | −3039 | −1260 | −465 | −2604 | −2536 | −3331 | −3001 | −2988 | 407 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 402(I) | −1761 | −1312 | −4317 | −3954 | −1713 | −4027 | −3703 | 3225 | −3814 | −556 | −498 | −3712 | −3859 | −3653 | −3877 | −3344 | −1754 | 2110 | −3216 | −2787 | 408 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 403(S) | −348 | −981 | −2200 | −2194 | −2989 | −1227 | −2073 | −2686 | −2157 | −2970 | −2136 | −1541 | −1946 | −1946 | −2253 | 3060 | 1398 | −1824 | −3217 | −2916 | 409 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 404(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 410 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 405(V) | −917 | −809 | −2556 | −1976 | −827 | −2491 | −1367 | 1339 | 1455 | 721 | 94 | −1841 | −2501 | −1487 | −1710 | −1570 | −863 | 2038 | −1514 | −1151 | 411 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 406(K) | −1386 | −2643 | −447 | 1824 | −3108 | −1893 | −570 | −2762 | 2860 | −2616 | −1848 | −552 | −2117 | −166 | −3 | −1217 | −1300 | −2388 | −2647 | −2154 | 412 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 407(N) | −537 | −1563 | −449 | −36 | −1889 | 1143 | −307 | −1529 | 932 | −1655 | −844 | 1794 | −1658 | 73 | −356 | −518 | −516 | 924 | −1962 | −1392 | 413 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 408(P) | −894 | −2181 | −369 | 1705 | −2576 | −1650 | −357 | −2268 | 243 | −2210 | −1375 | −330 | 2093 | 63 | 1619 | −774 | −835 | −1876 | −2347 | −1769 | 414 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 409(V) | −419 | −634 | −1376 | −807 | 1053 | −1737 | −499 | −198 | −623 | −505 | 178 | 600 | −1807 | −475 | 475 | 313 | −360 | 1389 | −1016 | 1303 | 415 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 410(I) | −1282 | −1082 | −3022 | −2555 | 2426 | −2683 | 1767 | 2555 | −2191 | −443 | −88 | −2038 | −2692 | −1794 | −2075 | −1793 | −1220 | −317 | −361 | 552 | 416 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 411(T) | −499 | −1595 | −431 | 966 | −1830 | −1487 | −185 | −1449 | 1092 | −1574 | −754 | −207 | −1601 | 213 | −206 | −458 | 2067 | 159 | −1877 | −1296 | 417 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 412(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 418 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 413(P) | −632 | −1230 | −2074 | −2144 | −2996 | −1453 | −2116 | −2631 | −2128 | −2928 | −2213 | −1658 | 3610 | −2006 | −2221 | −852 | 1302 | −1931 | −3185 | −2917 | 419 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 414(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 420 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 415(R) | −1454 | −2316 | −1780 | −878 | −2834 | −2232 | −428 | −2292 | 2281 | −2200 | −1473 | −940 | −2240 | −17 | 2627 | −1386 | −1270 | 588 | −2249 | −1960 | 421 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 416(V) | −1771 | −1603 | −3750 | −3689 | −2037 | −3050 | −3231 | 403 | −3479 | −1154 | −1076 | −3246 | −3399 | −3383 | −3437 | −2628 | −1917 | 3536 | −3074 | −2677 | 422 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 417(F) | −3342 | −2776 | −4026 | −4232 | 4354 | −3545 | −1431 | −2315 | −4038 | −1801 | −1900 | −3299 | −3780 | −3350 | −3645 | −3490 | −3420 | −2566 | −739 | 349 | 423 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 418(D) | −1572 | −3426 | 2573 | 2447 | −3613 | −1583 | −879 | −3513 | −1050 | −3393 | −2684 | 1292 | −2085 | −535 | −1855 | −1253 | −1623 | −3000 | −3585 | −2609 | 424 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 419(S) | −879 | −1989 | 1498 | −177 | −3045 | 1600 | −939 | −2843 | −904 | −2867 | −2046 | −438 | −1922 | −591 | −1483 | 2171 | −1044 | −2226 | −3072 | −2372 | 425 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 420(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 426 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 421(Q) | −705 | −1925 | −199 | 2112 | 917 | −1534 | −288 | −1824 | 42 | −1842 | −1054 | −210 | −1709 | 2163 | −420 | −611 | −656 | −1502 | −1997 | −1291 | 427 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 422(H) | −569 | −2048 | 1450 | 1526 | −2349 | −1405 | 1830 | −2103 | 181 | −2058 | −1157 | −37 | −1569 | 272 | −349 | 713 | 620 | −1662 | −2240 | −1537 | 428 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 423(C) | 1626 | 2878 | −2671 | −2107 | 1264 | −1968 | −1091 | 233 | −1777 | −334 | 250 | −1672 | −2128 | −1459 | −1691 | −1096 | −529 | 1209 | −1066 | −704 | 429 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 424(M) | −2042 | −1634 | −4379 | −3826 | −659 | −3976 | −2899 | 2765 | −3546 | 1204 | 3085 | −3605 | −3604 | −2896 | −3318 | −3183 | −1961 | 195 | −2135 | −2058 | 430 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 425(E) | 412 | −2447 | 1356 | 2379 | −2747 | −1477 | −445 | −2527 | −243 | −2477 | −1622 | −107 | 855 | −36 | −831 | −730 | −894 | −2073 | −2668 | −1906 | 431 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 426(A) | 2822 | −1031 | −2418 | −2539 | −3226 | 1898 | −2364 | −2941 | −2626 | −3229 | −2379 | −1722 | −2026 | −2302 | −2634 | −654 | −848 | −1983 | −3415 | −3226 | 432 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 427(I) | −1772 | −1325 | −4307 | −3877 | −1405 | −3993 | −3383 | 2935 | −3705 | 820 | −217 | −3632 | −3761 | −3400 | −3682 | −3260 | −1742 | 2033 | −2838 | −2525 | 433 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 428(L) | −875 | −1634 | −575 | 959 | −1581 | −1769 | −525 | −1179 | −135 | 1884 | −625 | −547 | −1931 | 1405 | −450 | −909 | −816 | −1074 | −1883 | −1383 | 434 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 429(A) | 1705 | −1826 | −180 | 949 | −2318 | −1410 | −359 | −2041 | −53 | −2067 | −1204 | 1001 | −1652 | 52 | −561 | 1232 | −595 | −1609 | −2298 | −1643 | 435 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 430(D) | −1074 | −2458 | 2381 | 60 | −2921 | 1927 | −658 | −2710 | −463 | −2675 | −1860 | −271 | −1918 | −276 | 866 | −915 | −1100 | −2245 | −2845 | −2124 | 436 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 431(K) | −688 | −2117 | 785 | 888 | −2469 | −1529 | −187 | −2189 | 2380 | −2106 | −1221 | −162 | −1661 | 256 | 1134 | −553 | −619 | −1760 | −2240 | −1607 | 437 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 432(I) | −2019 | −1582 | −4380 | −3941 | −1000 | −4086 | −3253 | 3295 | −3671 | 1100 | 145 | −3736 | −3783 | −3222 | −3556 | −3378 | −1976 | 657 | −2517 | −2289 | 438 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 433(Q) | −490 | −1797 | −369 | 171 | −2078 | −1457 | 1762 | −1779 | 1157 | −1780 | −905 | 1165 | −1550 | 1798 | −48 | −396 | −422 | 725 | −1986 | −1366 | 439 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 434(A) | 1954 | −1836 | 1733 | −180 | −2714 | −1429 | −806 | −2438 | −679 | −2518 | −1698 | −430 | 1775 | −448 | −1211 | −736 | −894 | −1923 | −2765 | −2117 | 440 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 435(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 441 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 436(D) | −1736 | −3455 | 3490 | 97 | −3737 | −1646 | −1070 | −3753 | −1363 | −3647 | −3016 | 1602 | −2204 | −760 | −2218 | −1420 | −1838 | −3213 | −3756 | −2780 | 442 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 437(V) | −1721 | −1302 | −4229 | −3874 | −1705 | −3894 | −3582 | 1607 | −3706 | −582 | −513 | −3610 | −3786 | −3559 | −3767 | −3209 | −1725 | 3294 | −3158 | −2712 | 443 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 438(V) | 594 | −988 | −3391 | −2911 | −1164 | −2888 | −2187 | 845 | −2637 | 765 | −154 | −2576 | −2962 | −2387 | −2622 | −2074 | −1205 | 2800 | −2084 | −1724 | 444 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 439(V) | −1771 | −1603 | −3750 | −3689 | −2037 | −3050 | −3231 | 403 | −3479 | −1154 | −1076 | −3246 | −3399 | −3383 | −3437 | −2628 | −1917 | 3536 | −3074 | −2677 | 445 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 440(I) | −1754 | −1308 | −4295 | −3867 | −1434 | −3978 | −3377 | 2661 | −3697 | 862 | −247 | −3617 | −3754 | −3406 | −3679 | −3243 | −1725 | 2373 | −2852 | −2526 | 446 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 441(R) | −2957 | −3022 | −3318 | −2735 | −3796 | −2998 | −1968 | −3912 | −846 | −3631 | −3157 | −2611 | −3280 | −1724 | 4056 | −3026 | −2913 | −3650 | −3096 | −3185 | 447 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 442(Y) | −1321 | −1438 | −1994 | −1608 | 2186 | 527 | −450 | −1117 | −1481 | −1211 | −693 | 1178 | −2522 | −1217 | −1665 | −1518 | −1275 | −1021 | −198 | 3178 | 448 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 443(C) | −675 | 2205 | −2544 | 972 | −572 | −2236 | −1121 | 1373 | −1671 | 679 | 261 | −1700 | −2270 | −1403 | −1668 | −1311 | −621 | 1601 | −1150 | −790 | 449 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 444(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3658 | −4222 | 450 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 445(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 451 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 446(K) | −1060 | −2058 | −1088 | −460 | −2432 | −1917 | −357 | −1970 | 2801 | −1978 | −1220 | −632 | −1990 | 1339 | 367 | −999 | −946 | 536 | −2145 | −1717 | 452 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 447(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 453 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 448(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 454 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 449(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 455 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 450(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 456 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 451(M) | −2406 | −2296 | −3638 | −3594 | −1525 | −3105 | −2824 | −1047 | −3121 | −596 | 5043 | −3293 | −3425 | −3046 | −2996 | −2911 | −2552 | −1398 | −2513 | −2207 | 457 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 452(P) | −1659 | −2241 | −2022 | −1646 | −3185 | −2242 | −1373 | −3000 | −450 | −2936 | −2274 | −1624 | 3435 | −1065 | 2095 | −1730 | −1750 | −2593 | −2816 | −2613 | 458 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 453(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 459 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 454(M) | −2406 | −2296 | −3638 | −3594 | −1525 | −3105 | −2824 | −1047 | −3121 | −596 | 5043 | −3293 | −3425 | −3046 | −2996 | −2911 | −2552 | −1398 | −2513 | −2207 | 460 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 455(L) | −2871 | −2457 | −4231 | −4103 | −1033 | −3803 | −3165 | −541 | −3734 | 3130 | −31 | −3935 | −3797 | −3286 | −3484 | −3713 | −2869 | −1136 | −2394 | −2220 | 461 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 456(K) | 1368 | −1491 | −763 | −332 | −2319 | −1417 | −551 | −1998 | 1786 | −2068 | −1221 | −500 | −1721 | −160 | −470 | 1631 | −587 | −1532 | −2299 | −1754 | 462 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 457(P) | −1500 | −1738 | −2514 | −2380 | −1555 | −2358 | −2022 | −1126 | −2063 | 1224 | −841 | −2189 | 3436 | −2061 | −2129 | −1822 | −1674 | −1231 | −2290 | −1878 | 463 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 458(T) | −351 | −974 | −2208 | −2185 | −2894 | −1237 | −2041 | −2561 | −2125 | −2863 | −2046 | −1539 | −1948 | −1923 | −2218 | 1543 | 3230 | −1758 | −3139 | −2834 | 464 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 459(S) | −897 | −1462 | −2333 | −2543 | −3185 | −1640 | −2474 | −3294 | −2686 | −3497 | −2780 | −1973 | −2360 | −2483 | −2703 | 3465 | −1316 | −2413 | −3310 | −3025 | 465 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 460(M) | 2706 | −986 | −2433 | −2144 | −1502 | −1684 | −1706 | −700 | −1858 | −968 | 2744 | −1705 | −2188 | −1713 | −1932 | −963 | −862 | −592 | −2145 | −1794 | 466 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 461(I) | −2103 | −1659 | −4461 | −3992 | −869 | −4152 | −3233 | 3082 | −3723 | 1619 | 290 | −3801 | −3788 | −3171 | −3557 | −3432 | −2046 | 487 | −2418 | −2265 | 467 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 462(I) | −1761 | −1312 | −4317 | −3954 | −1713 | −4027 | −3703 | 3225 | −3814 | −556 | −498 | −3712 | −3859 | −3653 | −3877 | −3344 | −1754 | 2110 | −3216 | −2787 | 468 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 463(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 469 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 464(K) | 1641 | −2033 | −323 | 914 | −2415 | −1565 | −296 | −2097 | 2052 | −2080 | −1233 | −257 | −1736 | 125 | −133 | −646 | −702 | −1707 | −2258 | −1657 | 470 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 465(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 471 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 466(L) | −1699 | −1807 | −2268 | −1925 | −830 | −2795 | −1551 | −455 | −1225 | 2510 | 90 | −1958 | −2845 | 1927 | −1308 | −2067 | −1651 | −846 | −1841 | −1454 | 472 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 467(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | 4222 | 473 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 468(D) | −853 | −2415 | 2115 | 1717 | −2702 | −1468 | −378 | −2484 | 1085 | −2417 | −1546 | −84 | −1732 | 41 | −699 | 696 | −824 | −2025 | −2594 | −1839 | 474 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 469(S) | −892 | −1780 | −931 | −688 | −2757 | −1643 | −830 | −2472 | 1671 | −2492 | −1708 | −799 | −2018 | −468 | −365 | 2676 | −1004 | −1981 | −2598 | −2130 | 475 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 470(C) | −1135 | 3503 | −3700 | −3406 | −1670 | −2549 | −2675 | 653 | −3101 | −916 | −667 | −2727 | −2925 | −2870 | −3030 | −1868 | −1288 | 2927 | −2619 | −2222 | 476 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 471(A) | 2590 | −1035 | −2404 | −2530 | −3236 | 2290 | −2365 | −2954 | −2627 | −3240 | −2389 | −1719 | −2027 | −2302 | −2637 | −656 | −851 | −1991 | −3423 | −3234 | 477 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 472(L) | −2632 | −2152 | −4630 | −4185 | 1767 | −4324 | −2442 | −61 | −3879 | 2789 | 563 | −3833 | −3823 | −2970 | −3513 | −3609 | −2518 | −738 | −1527 | −945 | 478 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 473(I) | −2073 | −1632 | −4434 | −3975 | −911 | −4130 | −3238 | 3164 | −3706 | 1451 | 244 | −3779 | −3785 | −3187 | −3557 | −3413 | −2021 | 546 | −2449 | −2273 | 479 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 474(T) | −1213 | −1674 | −2755 | −2906 | −3163 | −1922 | −2659 | −2698 | −2788 | −3105 | −2612 | −2311 | −2600 | −2708 | −2753 | −1463 | 3819 | −2197 | −3286 | −3156 | 480 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 475(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 481 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 476(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 482 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 477(R) | −2957 | −3022 | −3318 | −2735 | −3796 | −2998 | −1968 | −3912 | −846 | −3631 | −3157 | −2611 | −3280 | −1724 | 4056 | −3026 | −2913 | −3650 | −3096 | −3185 | 483 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 478(F) | −3342 | −2776 | −4026 | −4232 | 4354 | −3545 | −1431 | −2315 | −4038 | −1801 | −1900 | −3299 | −3780 | −3350 | −3645 | −3490 | −3420 | −2566 | −739 | 349 | 484 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 479(S) | −897 | −1462 | −2333 | −2543 | −3185 | −1640 | −2474 | −3294 | −2686 | −3497 | −2780 | −1973 | −2360 | −2483 | −2703 | 3465 | −1316 | −2413 | −3310 | −3025 | 485 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 480(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | 4222 | 486 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 481(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 487 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 482(T) | −359 | −976 | −2225 | −2229 | −2900 | −1242 | −2074 | −2560 | −2170 | −2875 | −2064 | −1561 | −1958 | −1969 | −2247 | 1110 | 3375 | −1760 | −3152 | −2850 | 488 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 483(Y) | −3402 | −2632 | −3941 | −4011 | 1064 | −3924 | 3388 | −2526 | −3541 | −1996 | −1973 | −2625 | −3821 | −2664 | −3170 | −3135 | −3280 | −2619 | 3420 | 3756 | 489 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 484(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 490 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 485(M) | −2322 | −1904 | −4536 | −3951 | 2387 | −4112 | −2676 | 67 | −3649 | 2034 | 3156 | −3710 | −3633 | −2803 | −3311 | −3309 | −2204 | −588 | −1794 | −1586 | 491 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 486(V) | −1771 | −1603 | −3750 | −3689 | −2037 | −3050 | −3231 | 403 | −3479 | −1154 | −1076 | −3246 | −3399 | −3383 | −3437 | −2628 | −1917 | 3536 | −3074 | −2677 | 492 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 487(V) | −1771 | −1603 | −3750 | −3689 | −2037 | −3050 | −3231 | 403 | −3479 | −1154 | −1076 | −3246 | −3399 | −3383 | −3437 | −2628 | −1917 | 3536 | −3074 | −2677 | 493 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 488(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 494 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 489(H) | −3205 | −3079 | −2723 | −2890 | −2110 | −3046 | 5295 | −4135 | −2617 | −3813 | −3561 | −2886 | −3482 | −2833 | −2620 | −3291 | −3356 | −3895 | −2397 | −1681 | 495 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 490(V) | −1754 | −1297 | −4329 | −3968 | −1770 | −4053 | −3752 | 2604 | −3840 | −621 | −545 | −3728 | −3878 | −3699 | −3917 | −3370 | −1746 | 2859 | −3276 | −2829 | 496 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 491(A) | 2587 | −828 | −2477 | −2155 | −1837 | −1468 | −1728 | −743 | −1941 | −1564 | −954 | −1607 | −2033 | −1725 | −2034 | −738 | 1178 | 1108 | −2310 | −1972 | 497 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 492(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | 4064 | 498 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 493(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 499 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 494(A) | 3438 | −1472 | −2846 | −3040 | −3287 | −1726 | −2735 | −2840 | −3028 | −3257 | −2662 | −2235 | −2447 | −2798 | −2944 | −1216 | −1387 | −2183 | −3405 | −3320 | 500 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 495(Y) | −866 | −976 | −1863 | −1331 | 1353 | −2145 | 1318 | −556 | −1116 | −777 | −173 | −1242 | −2197 | 1714 | −1301 | −1173 | −802 | 888 | −445 | 2749 | 501 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 496(D) | 417 | −1831 | 1647 | 1094 | −2065 | −1488 | −353 | −1618 | −107 | −1820 | −1019 | −189 | −1698 | 30 | −623 | −603 | −643 | 1629 | −2154 | −1520 | 502 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 497(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | 4222 | 503 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 498(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | 4222 | 504 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 499(T) | 492 | −1190 | −706 | −181 | −1475 | 311 | −333 | −1099 | −81 | 71 | −509 | 570 | 1113 | −6 | −509 | −450 | 1123 | −835 | −1680 | −1161 | 505 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 500(I) | −2091 | −1746 | −3971 | −3840 | −1676 | −3532 | −3289 | 3684 | −3581 | −659 | −693 | −3562 | −3674 | −3445 | −3521 | −3194 | −2146 | 449 | −2877 | −2493 | 506 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 501(A) | 3103 | −1036 | −2445 | −2572 | −3222 | 1051 | −2380 | −2930 | −2650 | −3226 | −2381 | −1739 | −2034 | −2327 | −2648 | −664 | −857 | −1981 | −3412 | −3228 | 507 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 502(L) | −2239 | −1892 | −3711 | −3400 | 301 | −3520 | −1210 | −542 | −2948 | 2564 | −35 | −2786 | −3395 | −2438 | −2750 | −2747 | −2165 | −945 | −573 | 2562 | 508 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 503(V) | −1757 | −1387 | −4101 | −3681 | −1174 | −3714 | −3031 | 880 | −3410 | 1254 | −60 | −3407 | −3585 | −3094 | −3354 | −2984 | −1743 | 3014 | −2536 | −2219 | 509 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 504(Q) | −982 | −2251 | −866 | 971 | −2711 | −1822 | −252 | −2340 | 1444 | −2194 | −1356 | −464 | −1885 | 2646 | 1632 | −858 | −863 | −1958 | −2245 | −1765 | 510 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 505(E) | −1162 | −2771 | 2137 | 2239 | −3046 | −1526 | −626 | −2849 | −546 | −2792 | −1983 | −145 | −1905 | −242 | −1192 | −940 | 1396 | −2385 | −2990 | −2169 | 511 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 506(G) | −1707 | −2684 | 1591 | −614 | −3783 | 3190 | −1613 | −3795 | −1887 | −3775 | −3119 | −915 | −2456 | −1358 | −2539 | −1610 | −1924 | −3150 | −3636 | −3124 | 512 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 507(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 513 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 508(M) | −473 | −522 | −1819 | −1236 | −468 | −1879 | −687 | 1519 | −996 | 566 | 1677 | −1154 | −1937 | 836 | −1131 | 1079 | −413 | 102 | −957 | −585 | 514 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 509(I) | −1761 | −1312 | −4317 | −3954 | −1713 | −4027 | −3703 | 3225 | −3814 | −556 | −498 | −3712 | −3859 | −3653 | −3877 | −3344 | −1754 | 2110 | −3216 | −2787 | 515 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 510(T) | 782 | −1467 | −550 | 1029 | −2202 | −1425 | −709 | −1791 | −472 | −1993 | −1203 | −528 | −1787 | −368 | −902 | −617 | 2685 | −1400 | −2333 | −1783 | 516 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 511(I) | −1766 | −1333 | −4283 | −3923 | −1635 | −3967 | −3619 | 3388 | −3759 | −473 | −437 | −3672 | −3822 | −3576 | −3804 | −3285 | −1764 | 1695 | −3126 | −2717 | 517 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 512(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 518 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 513(A) | 2705 | −1451 | −1036 | −913 | −2506 | −1504 | −1143 | −2174 | −794 | −2337 | −1613 | −946 | −1993 | 2040 | −1061 | −809 | −910 | −1703 | −2633 | −2156 | 519 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 514(H) | −615 | −1680 | 1444 | 66 | −1883 | 168 | 2650 | −1558 | −86 | −1691 | −891 | −223 | −1680 | 31 | −577 | −571 | −585 | 1267 | −2007 | −1397 | 520 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 515(K) | −654 | −2006 | −546 | 42 | −2376 | −1581 | −133 | −2066 | 1935 | −1987 | −1107 | 1132 | −1658 | 1043 | 1058 | −540 | 1180 | −1660 | −2113 | −1532 | 521 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 516(N) | −933 | −2085 | −946 | −284 | −2472 | −1822 | −253 | −2090 | 1711 | 76 | −1204 | 1918 | −1876 | 175 | 1799 | −841 | −817 | −1755 | −2132 | −1663 | 522 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 517(E) | −416 | −987 | −843 | 1107 | −1070 | −1583 | −338 | −623 | −183 | 879 | −172 | −489 | −1679 | −94 | −565 | 544 | 813 | 265 | −1379 | −905 | 523 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 518(I) | −2258 | −1804 | −4588 | −4084 | −706 | −4269 | −3231 | 2527 | −3807 | 2292 | 465 | −3923 | −3814 | −3118 | −3570 | −3544 | −2181 | 190 | −2303 | −2237 | 524 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 519(Q) | −477 | −1909 | 958 | 282 | −2211 | −1389 | 1484 | −1953 | 285 | −1921 | −1018 | −32 | −1517 | 2318 | −225 | 630 | 559 | −1525 | −2110 | −1430 | 525 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 520(L) | −2127 | −1743 | −4402 | −3796 | 1257 | −3918 | −2674 | 149 | −3492 | 2527 | 2164 | −3553 | −3509 | −2714 | −3181 | −3095 | −2019 | 570 | −1870 | −1818 | 526 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 521(N) | −723 | −2217 | 958 | 236 | −2518 | −1456 | 1611 | −2279 | 1719 | −2217 | −1334 | 2285 | −1666 | 166 | −401 | −570 | −677 | −1837 | −2382 | −1678 | 527 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 522(V) | −1754 | −1297 | −4330 | −3968 | −1770 | −4053 | −3752 | 2623 | −3841 | −620 | −545 | −3729 | −3878 | −3699 | −3918 | −3371 | −1746 | 2846 | −3277 | −2830 | 528 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 523(S) | 1545 | −974 | −2003 | −1825 | −2867 | −1206 | −1790 | −2580 | −1788 | −2795 | −1932 | −1362 | 1826 | −1586 | −1999 | 2362 | −672 | −1755 | −3057 | −2721 | 529 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 524(D) | −1776 | −3649 | 3326 | 1869 | −3838 | −1642 | −1031 | −3788 | −1322 | −3660 | −3029 | −245 | −2192 | −711 | −2201 | −1425 | −1855 | −3264 | −3821 | −2816 | 530 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 525(E) | 423 | −2950 | 1944 | 2696 | −3223 | −1545 | −718 | −3047 | −715 | −2979 | −2196 | −161 | −1968 | −347 | −1403 | −1043 | −1314 | −2569 | −3177 | −2316 | 531 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 526(E) | −2641 | −3308 | −896 | 3732 | −3966 | −2458 | −2043 | −4105 | −2128 | −4016 | −3555 | −1531 | −2959 | −1842 | −2560 | −2479 | −2750 | −3722 | −3563 | −3385 | 532 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 527(L) | −2339 | −1899 | −4618 | −4042 | 1570 | −4204 | −2849 | 1440 | −3758 | 2558 | 676 | −3825 | −3700 | −2902 | −3418 | −3418 | −2226 | −382 | −1924 | −1778 | 533 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 528(A) | 2338 | −1990 | −241 | 938 | −2395 | −1557 | −423 | −2061 | 954 | −2103 | −1286 | −301 | −1791 | −26 | −375 | −717 | −784 | −1691 | −2330 | −1728 | 534 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 529(R) | 524 | −2098 | −789 | −146 | −2504 | −1729 | 1632 | −2153 | 1229 | −2054 | −1204 | −379 | −1789 | 1328 | 2313 | −719 | −724 | −1774 | −2150 | −1637 | 535 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 530(R) | −2957 | −3022 | −3318 | −2735 | −3796 | −2998 | −1968 | −3912 | −846 | −3631 | −3157 | −2611 | −3280 | −1724 | 4056 | −3026 | −2913 | −3650 | −3096 | −3185 | 536 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 531(R) | −1895 | −2713 | −2327 | −1192 | −3484 | −2502 | −481 | −2856 | 2144 | −2544 | −1842 | −1161 | −2458 | 1393 | 3023 | −1770 | −1619 | −2599 | −2421 | −2259 | 537 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 532(A) | 2935 | −1714 | −553 | 857 | −2769 | −1546 | −1218 | −2333 | −1106 | −2591 | −1873 | −809 | −2065 | −934 | −1502 | −954 | −1103 | −1872 | −2898 | −2374 | 538 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 533(A) | 1291 | −1874 | −176 | 1227 | −2177 | −1392 | −109 | −1909 | 277 | −1891 | −995 | 1134 | −1522 | 1248 | −228 | −361 | 562 | −1492 | −2090 | −1419 | 539 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 534(W) | −805 | −687 | −2581 | −2028 | 138 | −2236 | −697 | 897 | −1681 | −421 | 141 | −1645 | −2282 | −1369 | −1627 | −1315 | 636 | −90 | 4479 | 1809 | 540 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 535(H) | −408 | −1801 | −274 | 1284 | −2096 | −1385 | 1500 | −1822 | 1168 | −1802 | −899 | −33 | −1479 | 1381 | −102 | −303 | 595 | 221 | −1996 | −1339 | 541 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 536(Q) | −650 | −1737 | −627 | −72 | −1981 | −1615 | −209 | −1625 | 1223 | 392 | −866 | −318 | 1222 | 2120 | 50 | −598 | −572 | −1326 | −1932 | −1394 | 542 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 537(P) | −2931 | −2878 | −3420 | −3706 | −4181 | −2925 | −3468 | −4621 | −3859 | −4490 | −4165 | −3491 | 4225 | −3781 | −3695 | −3182 | −3279 | −4087 | −3594 | −4064 | 543 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −324 | −7108 | −2368 | −894 | −1115 | −701 | −1378 | * | * | | | | | | | | | | | | |
| 538(A) | 2195 | −924 | −968 | −546 | −1397 | −1356 | −583 | −812 | −365 | −1167 | −487 | −618 | −1660 | 1324 | −684 | 483 | −404 | 462 | −1703 | −1242 | 544 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −19 | −6804 | −7846 | −894 | −1115 | −428 | −1961 | * | * | | | | | | | | | | | | |
| 539(P) | 411 | −1017 | −1886 | −1616 | −1600 | −1588 | −1411 | −962 | −1408 | 495 | −755 | −1384 | 3156 | −1323 | −1577 | −847 | −785 | −783 | −2111 | −1716 | 545 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 540(R) | −1612 | −2397 | −2037 | −1033 | −2897 | −2352 | −458 | −2365 | 2184 | 665 | −1520 | −1051 | −2334 | −51 | 2602 | −1545 | −1395 | −2143 | −2262 | −2014 | 546 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 541(Y) | 712 | −796 | −2334 | −1883 | −370 | −2028 | −986 | −143 | −1607 | −663 | −131 | −1587 | −2243 | −1383 | −1656 | −1178 | −771 | 1114 | −965 | 3479 | 547 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 542(T) | −527 | −1669 | 1091 | −27 | −2315 | −1379 | −443 | −2033 | −151 | −2081 | −1218 | −282 | 557 | −41 | −650 | 1128 | 2077 | −1576 | −2321 | −1690 | 548 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 543(R) | −2957 | −3022 | −3318 | −2735 | −3796 | −2998 | −1968 | −3912 | −846 | −3631 | −3157 | −2611 | −3280 | −1724 | 4056 | −3026 | −2913 | −3650 | −3096 | −3185 | 549 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 544(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 550 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 545(V) | −1747 | −1296 | −4310 | −3948 | −1758 | −4023 | −3716 | 2215 | −3813 | −615 | −540 | −3705 | −3860 | −3670 | −3887 | −3339 | −1741 | 3087 | −3252 | −2806 | 551 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 546(L) | −2871 | −2457 | −4231 | −4103 | −1033 | −3803 | −3165 | −541 | −3734 | 3130 | −31 | −3935 | −3797 | −3286 | −3484 | −3713 | −2869 | −1136 | −2394 | −2220 | 552 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 547(A) | 2404 | −890 | −1926 | −1629 | −1803 | 1275 | −1415 | −1282 | −1490 | 392 | −963 | −1316 | −1930 | −1328 | −1674 | −654 | −644 | −952 | −2187 | −1810 | 553 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 548(K) | −2620 | −2961 | −2461 | −2046 | −3743 | −2791 | −1570 | −3603 | 3784 | −3387 | −2839 | −2048 | −3039 | −1260 | −465 | −2604 | −2536 | −3331 | −3001 | −2988 | 554 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 549(Y) | −3621 | −2707 | −4176 | −4424 | 2950 | −4049 | −394 | −2539 | −4002 | −1942 | −1987 | −2749 | −3933 | −2854 | −3451 | −3299 | −3499 | −2690 | 349 | 4094 | 555 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 550(A) | 3438 | −1472 | −2846 | −3040 | −3287 | −1726 | −2735 | −2840 | −3028 | −3257 | −2662 | −2236 | −2447 | −2798 | −2944 | −1216 | −1387 | −2183 | −3405 | −3320 | 556 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 551(H) | −1741 | −2627 | −2070 | −1046 | −3303 | −2401 | 2713 | −2751 | 2478 | −2476 | −1755 | −1061 | −2375 | −27 | 2379 | −1621 | −1497 | −2477 | −2379 | −2161 | 557 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 552(L) | −1014 | −876 | −2956 | −2408 | −582 | −2550 | −1529 | 1721 | −2079 | 2042 | 345 | −2114 | −2581 | −1775 | −2028 | 454 | −980 | 286 | −1414 | −1096 | 558 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 553(V) | 933 | −842 | −2818 | −2467 | −1542 | −1870 | −1890 | 154 | −2226 | −1095 | −617 | −1932 | −2326 | −1995 | −2259 | −1126 | 1070 | 2769 | −2180 | −1826 | 559 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 554(S) | −787 | −1522 | −1486 | −1172 | −2714 | −1599 | −1112 | −2500 | −433 | −2563 | −1791 | −1110 | −2067 | −796 | 1351 | 2916 | −989 | −1943 | −2648 | −2234 | 560 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 555(S) | −326 | −1010 | −1779 | −1541 | −2691 | −1234 | −1566 | −2386 | −1486 | −2594 | −1749 | −1228 | 1196 | −1330 | −1747 | 2396 | 1967 | −1662 | −2876 | −2496 | 561 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 556(A) | 3121 | −934 | −2489 | −2561 | −3081 | −1203 | −2295 | −2766 | −2533 | −3080 | −2234 | −1669 | −1953 | −2234 | −2533 | 936 | −746 | −1844 | −3331 | −3090 | 562 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 557(S) | −897 | −1462 | −2333 | −2543 | −3185 | −1640 | −2474 | −3294 | −2686 | −3497 | −2780 | −1973 | −2360 | −2483 | −2703 | 3465 | −1316 | −2413 | −3310 | −3025 | 563 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 558(R) | −586 | −1873 | −516 | 979 | −2188 | −1543 | −123 | −1869 | 1290 | −353 | −980 | −202 | −1622 | 314 | 1886 | −491 | 782 | −1495 | −2024 | −1439 | 564 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 559(G) | −2594 | −2690 | −3304 | −3623 | −4328 | 3747 | −3462 | −4761 | −3953 | −4671 | −4212 | −3320 | −3352 | −3748 | −3779 | −2839 | −2981 | −4004 | −3668 | −4222 | 565 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 560(C) | 2804 | 3772 | −3185 | −3198 | −2739 | −1303 | −2462 | −2065 | −2882 | −2628 | −1924 | −1927 | −2044 | −2547 | −2727 | −661 | −799 | −1463 | −3099 | −2886 | 566 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 561(V) | −1771 | −1603 | −3750 | −3689 | −2037 | −3050 | −3231 | 403 | −3479 | −1154 | −1076 | −3246 | −3399 | −3383 | −3437 | −2628 | −1917 | 3536 | −3074 | −2677 | 567 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 562(T) | −1213 | −1674 | −2755 | −2906 | −3163 | −1922 | −2659 | −2698 | −2788 | −3105 | −2612 | −2311 | −2600 | −2708 | −2753 | −1463 | 3819 | −2197 | −3286 | −3156 | 568 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −16 | −7108 | −8150 | −894 | −1115 | −701 | −1378 | * | * | | | | | | | | | | | | |
| 563(D) | −2784 | −3432 | 4016 | −1200 | −4140 | −2466 | −2197 | −4505 | −2621 | −4365 | −3956 | −1551 | −3014 | −2039 | −3232 | −2593 | −2938 | −4046 | −3710 | −3552 | 569 |
| — | −149 | −500 | 233 | 43 | −381 | 399 | 106 | −626 | 210 | −466 | −720 | 275 | 394 | 45 | 96 | 359 | 117 | −369 | −294 | −249 | |
| — | −21 | −6715 | −7757 | −894 | −1115 | −701 | −1378 | * | * | | | | | | | | | | | | |
| 564(F) | −525 | −445 | −2202 | −1627 | 1946 | −2001 | −744 | 1247 | −1346 | 952 | 561 | 1079 | −2030 | −1067 | −1362 | −1067 | −465 | 338 | −714 | −230 | 570 |
| — | * | * | * | * | * | * | * | * | * | * | * | * | * | * | * | * | * | * | * | * | |
| — | * | * | * | * | * | * | * | * | 0 | | | | | | | | | | | | |

What is claimed is:

1. A recombinant yeast host cell comprising a heterologous dihydroxy-acid dehydratase (DHAD) protein that comprises
(i) an amino acid sequence having at least 95% identity to SEQ ID NO: 179 or 187 using the Clustal W method of alignment using the default parameters of GAP PENALTY=10, GAP LENGTH PENALTY=0.1, and Gonnet 250 series of protein weight matrix over the full length of the protein sequence; and
(ii) three conserved cysteine residues that correspond to positions 56, 129, and 201 of SEQ ID NO:179;
wherein the substrate 2,3-dihydroxyisovalerate is present in the yeast host cell and wherein the expressed heterologous DHAD protein catalyzes the conversion of 2,3-dihydroxyisovalerate to α-ketoisovalerate.

2. The recombinant yeast host cell of claim 1, wherein the recombinant yeast host cell comprising the heterologous DHAD is transformed with a chimeric gene that encodes said heterologous DHAD.

3. The recombinant yeast host cell of claim 1, wherein the amino acid sequence of the heterologous DHAD protein comprises SEQ ID NO: 179.

4. The recombinant yeast host cell of claim 1, wherein the recombinant yeast host cell comprises an engineered isobutanol biosynthetic pathway.

5. The recombinant yeast host cell of claim 4, wherein the engineered isobutanol biosynthetic pathway comprises the following substrate to product conversions:
(i) pyruvate to acetolactate (pathway step a);
(ii) acetolactate of (i) to 2,3-dihydroxyisovalerate (pathway step b);
(iii) 2,3-dihydroxyisovalerate of (ii) to α-ketoisovalerate (pathway step c);
(iv) α-ketoisovalerate of (iii) to isobutyraldehyde (pathway step d); and
(v) isobutyraldehyde of (iv) to isobutanol (pathway step e), and wherein
(a) the substrate to product conversion of step (i) is catalyzed by an acetolactate synthase;
(b) the substrate to product conversion of step (ii) is catalyzed by a ketol-acid reductoisomerase;
(c) the substrate to product conversion of step (iii) is catalyzed by the heterologous DHAD;
(d) the substrate to product conversion of step (iv) is catalyzed by an α-keto acid decarboxylase; and
(e) the substrate to product conversion of step (v) is catalyzed by an alcohol dehydrogenase.

6. The recombinant yeast host cell of claim 5, wherein two or more of the enzymes (a), (b), and (d) are heterologous to the recombinant yeast host cell.

7. The recombinant yeast host cell of claim 6, wherein two or more of the enzymes (a), (b), and (d) are over-expressed in the recombinant yeast host cell.

8. A method of converting 2,3-dihydroxyisovalerate to α-ketoisovalerate, comprising:
(a) providing the recombinant yeast host cell of claim 2; and
(b) culturing the recombinant yeast host cell under suitable conditions where 2,3-dihydroxyisovalerate is converted into α-ketoisovalerate.

9. The method of claim 8, wherein the recombinant yeast host cell comprises a disruption in an ilv3 gene that encodes mitochondrial DHAD, resulting in reduced expression of endogenous mitochondrial DHAD.

10. A method of producing isobutanol, comprising:
(a) providing the recombinant yeast host cell of claim 1.

11. The method of claim 10, wherein the recombinant yeast host cell comprises a disruption in an ilv3 gene that encodes mitochondrial DHAD, resulting in reduced expression of endogenous mitochondrial DHAD.

12. The method of claim 10, wherein the recombinant yeast host cell comprises an engineered isobutanol biosynthetic pathway.

13. The method of claim 10, further comprising:
(b) culturing the recombinant yeast host cell under suitable conditions to produce isobutanol from pyruvate; and
(c) recovering the isobutanol.

14. The method of claim 13, wherein said recovering is by distillation, liquid-liquid extraction, adsorption, decantation, pervaporation or combinations thereof.

15. The method of claim 13, further comprising (d) removing solids from the fermentation medium.

16. The method of claim 15, wherein said removing is by centrifugation, filtration or decantation.

17. The method of claim 15, wherein said removing step (d) occurs before said recovering step (c).
